(19) United States
(12) Patent Application Publication
(10) Pub. No.: US 2011/0282056 A1
(43) Pub. Date: Nov. 17, 2011

(54) PROTEIN KINASE INHIBITORS AND USE THEREOF

(75) Inventors: Benny C. Askew, Marshfield, MA (US); Nadia Brugger, Cambridge, MA (US); Ruoxi Lan, Waltham, MA (US); Amanda E. Sutton, Hingham, MA (US)

(73) Assignee: MERCK PATENT GMBH, Darmstadt (DE)

(21) Appl. No.: 12/811,053

(22) PCT Filed: Jan. 20, 2009
(86) PCT No.: PCT/US09/00291

§ 371 Date: Aug. 16, 2010

Related U.S. Application Data
(60) Provisional application No. 61/011,872, filed on Jan. 22, 2008.

(51) Int. Cl. C07D 471/04; C07D 401/14; C07D 413/14; C07D 487/04; C07D 495/04

(52) U.S. Cl. 544/127; 546/113; 544/280; 546/122; 544/250; 544/333

(57) ABSTRACT

Disclosed are compounds according to Formula I:

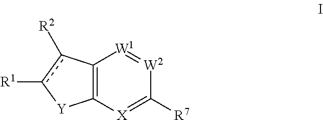

wherein the variables are described herein.

### BACKGROUND OF THE INVENTION

Protein kinases represent a large family of proteins, which play a central role in the regulation of a wide variety of cellular processes, maintaining control over cellular function. A partial list of such kinases includes Akt, Axl, Aurora A, Aurora B, dyrk2, epha2, fgfr3, igflr, IKK2, JNK3, VEGFR1, VEGFR2, VEGFR3 (also known as Flt-4), KDR, MEK1, MET, P70s6K, Plk1, RSK1, Src, TrkA, Zap70, cKit, bRaf, EGFR, Jak2, PI3K, NPM-Alk, c-Abl, BTK, FAK, PDGFR, TAK1, LimK, Flt-3, Flt-1, PDK1 and Erk Inhibition of such kinases has become an important therapeutic target.

Certain diseases are known to be associated with the deregulation of angiogenesis (growth of blood vessels) and/or lymphangiogenesis (growth of lymphatic vessels). Examples include lymphomas, ocular neovascularisation, corneal allograft rejection, age-related macular degeneration, psoriasis, hemangioblastoma, hemangioma, arteriosclerosis, inflammatory disease, arterial or posttransplantational arteriosclerosis, endometriosis, and neoplastic diseases, including solid tumors, liquid tumors (such as leukemias) and metastatic neoplastic disease.

Vascular endothelial growth factor receptors (VEGFR) are transmebranous receptor tyrosine kinases. They are characterized by an extracellular domain with seven immunoglobulin-like domains and an intracellular tyrosine kinase domain. Various types of VEGF receptors are known, e.g., VEGFR1 (also known as Flt-1), VEGFR2 (also known as KDR), and VEGFR3 (also known as Flt-4). VEGFR2 and VEGFR3 are expressed predominantly on blood vascular and lymphatic endothelia, respectively (Stacker, et al., FASEB J. 16: 9222-934, (2002)). VEGFR3 signals for lymphangiogenesis (Detmar, et al., Clin. Cancer Res., 12(23) 6865-6868 (2006)) while VEGFR2 is implicated in angiogenesis (Olsson, et al., Nature Reviews Molecular Cell Biology, 7, 359-371 (2006)).

The vascular endothelial growth factors (VEGFs) are dimeric glycoproteins of approximately 40 kDa and are integral regulators of vascular development during embryogenesis and blood vessel formation (angiogenesis) in the adult. VEGFR3 binds to two of the VEGFs, VEGF-C and VEGF-D. (Olsson, et al., Nature Reviews Molecular Cell Biology, 7, 359-371 (2006)).

Lymphatic vessels differ from blood vessels in several ways. Large collecting lymphatic vessels contain vascular smooth muscle cells in their walls, as well as valves, which prevent the backflow of lymph. However, lymphatic capillaries, unlike typical blood capillaries, lack pericytes and continuous basal lamina, and contain large inter-endothelial openings (Lohela, et al., Thromb. Haemost., 90:167 (2003)). Due to their greater permeability, lymphatic capillaries are more effective than blood capillaries in allowing tumor cells to pass. Thus, inhibitors of lymphangiogenesis play an important role in decreasing the migratory and invasive nature of tumor cells, decreasing the incidence of metastasis, and disrupting the formation of lymphatic vessels, which in turn decreases cell proliferation.

Tumor cell metastasis to regional lymph nodes is an early event in metastatic tumor spread. Tumor cell dissemination is mediated by a number of mechanisms among which are tissue invasion, lymphatic spread, haematogenous spread and direct seeding of body cavities or surfaces. Clinical and pathological observations suggest, for many tumors, the common pathway of initial dissemination is via the lymphatic system. The VEGFR3 signaling has a multifaceted role in tumor cell migration and invasion, lymphatic endothelial cell proliferation and migration, and endothelial cell proliferation and migration. In addition, VEGFR3 is associated with a variety of human malignancies such as lung adenocarcinoma, colon adenocarcinoma, head and neck carcinomas, prostate carcinoma, leukemia and Kaposi's sarcoma. (Su, J-L, et al., Cancer Cell, 9, 209-223 (2006)).

Angiogenesis is regarded as a prerequisite for tumors which grow beyond a diameter of about 1-2 mm Below 1-2 mm, oxygen and nutrients maybe supplied to the tumor cells by diffusion, however, tumors greater than 2 mm, regardless of its origin and its cause, are dependent on angiogenesis for its continued growth.

Three principal mechanisms play an important part in the activity on angiogenesis inhibitors against tumors: 1) inhibition of the growth of blood vessels, especially capillaries, into avascular resting tumors, which results in no net tumor growth owing to the balance that is achieved between cell death and proliferation; 2) prevention of the migration of tumor cells, which is caused by the absence of blood flow to and from the tumors; and 3) inhibition of endothelial cell proliferation, thus avoiding the paracrine growth-stimulating effect exerted on the surrounding tissue by the endothelial cells which normally line vessels. (R. Connell & J. Beebe, Exp. Opin. Ther. Patents, 11, 77-114 (2001)).

VEGFs are unique in that they are the only angiogenic growth factors known to contribute to the vascular hyperpermeability and edema that is associated with the expression or administration of many other growth factors. VEGF production appears to mediate the vascular hyperpermeability and edema processes. The production of VEGFs is stimulated by inflammatory cytokines such as IL-1 and tumor necrosis factor. Since many tumors (liquid and solid) express proinflammatory cytokines, and the central role of such cytokines is the regulation of the VEGF-C & VEGF-D, the signaling pathways of VEGF are of great therapeutic interest. Because angiogenesis and lymphangiogenesis have an important role in many human disease states, regulators of both angiogenesis and lymphangiogenesis have become important therapeutic targets.

### SUMMARY OF THE INVENTION

The invention provides in one aspect, compounds according to Formula I:

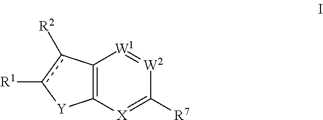

or a pharmaceutically acceptable salt thereof, wherein:

W¹ is CR⁸ or N, and W² is —C—C≡C—Ar; or W¹ is —C—C≡C—Ar, and W² is CR⁸ or N. Y is —S—, —O—, —NH—, or —NHCH₂—. X is N or N⁺—O⁻.

[symbol] represents either a single or a double bond.

Ar is aryl, carbocyclyl, heteroaryl or heterocyclyl; wherein the aryl and heteroaryl are optionally and independently substituted with up to 4 groups represented by R³, and wherein the carbocyclyl and heterocyclyl are optionally and independently substituted with up to 4 groups represented by R⁴.

R¹ and R² are independently selected from H, halogen, cyano, (C₁-C₆)alkyl, (C₂-C₆)alkenyl, (C₂-C₆)alkynyl, (C₁-C₆)alkoxy, (C₃-C₈)cycloalkyl, 5-10 membered heterocycloalkyl, —C(O)OR⁵, —C(O)R⁵, —OC(O)R⁵, —C(O)NR⁵R⁶, —NR⁵C(O)NR⁵R⁶, —NR⁵C(O)OR⁵, —NR⁵C(O)R⁵, —NR⁵C(S)NR⁵R⁶, —S(O)ₚNR⁵R⁶, —NR⁵R⁶, —S(O)ₚR⁵, —NR⁵S(O)ₚR⁵, wherein said alkyl, alkenyl, alkynyl, alkoxy, cycloalkyl, and heterocycloalkyl are each substituted or unsubstituted; or R¹ and R² can be taken together with their intervening atoms to form a (C₅-C₆)cycloalkyl ring which is substituted or unsubstituted; and p is an integer from 0 to 2.

Each R³ is independently: i) halogen, —X₁—OH, —X₁—CN, —X₁—OR¹⁰, —X₁—CO₂R¹⁰, —X₁—NR¹⁰C(O)N(R¹⁰)₂, —X₁—NR¹⁰CO₂R¹⁰, —X₁—COR¹⁰, —X₁—N(R¹⁰)₂, —X₁—N⁺ (R¹⁰)₃, —X₁—OCOR¹⁰, —X₁—SO₂N(R¹⁰)₂; —X₁—S(O)ₙR¹⁰; —X₁—NR¹⁰S(O)ₙR¹⁰, —X₁—NR¹⁰COR¹⁰, —X₁—OC(O)N(R¹⁰)₂, —X₁—CON(R¹⁰)₂ or —X₁—NR¹⁰CO₂R¹⁰; or ii) (C₁-C₆)alkyl, (C₁-C₆)haloalkyl, (C₂-C₆)alkenyl, (C₂-C₆)haloalkenyl, (C₂-C₆)alkynyl or (C₂-C₆)haloalkynyl; or iii) aryl, aralkyl, aryloxy, heteroaryl, heteroaralkyl, or heteroaryloxy, each optionally and independently substituted with up to 3 groups selected from halogen, —CN, —OH, —NH₂, —NH(C₁-C₆)alkyl, —N((C₁-C₆)alkyl)₂, (C₁-C₆)alkyl, halo(C₁-C₆)alkyl, (C₁-C₆)alkoxy, halo(C₁-C₆)alkoxy, (C₁-C₆)alkoxycarbonyl, —CONH₂, —CONH(C₁-C₆)alkyl, —CON((C₁-C₆)alkyl)₂, —CO(C₁-C₆)alkyl or —CO₂H; or iv) carbocyclyl or heterocyclyl, each optionally and independently substituted with up to 3 groups selected from halogen, —CN, —OH, —NH₂, —NH(C₁-C₆)alkyl, —N((C₁-C₆)alkyl)₂, (C₁-C₆)alkyl, halo(C₁-C₆)alkyl, (C₁-C₆)alkoxy, halo(C₁-C₆)alkoxy, (C₁-C₆)alkoxycarbonyl, —CONH₂, —CONH(C₁-C₆)alkyl, —CON((C₁-C₆)alkyl)₂, —CO(C₁-C₆)alkyl, —CO₂H, aryl, heteroaryl, oxo and thioxo.

Each X₁ is independently a covalent bond, a (C₁-C₆)alkylene, (C₁-C₆)alkenylene or (C₁-C₆)alkynylene.

Each R⁴ is independently a group represented by R³, oxo or thioxo.

The variable n is an integer from 0 to 2.

Each R⁵ and R⁶ are independently selected from H, (C₁-C₄)alkyl, (C₃-C₈)cycloalkyl or phenyl; wherein said alkyl, cycloalkyl and phenyl are optionally and independently substituted with halogen, —CN, —OH, —NH₂, —OCF₃, —OMe, or (C₁-C₃)alkyl.

R⁷ and R⁸ are independently H, halogen, cyano, (C₁-C₆)alkyl, (C₂-C₆)alkenyl, (C₂-C₆)alkynyl, —OR⁵, (C₃-C₈)cycloalkyl, 5-10 membered heterocycloalkyl, —C(O)OR⁵, —C(O)R⁵, —OC(O)R⁵, —C(O)NR⁵R⁶, —NR⁵C(O)NR⁵R⁶, —NR⁵C(O)OR⁵, —NR⁵C(O)R⁵, —NR⁵C(S)NR⁵R⁶, —S(O)ₚNR⁵R⁶, —NR⁵R⁶, —SR⁵, —NR⁵S(O)ₚR⁵, wherein said alkyl, alkenyl, alkynyl, cycloalkyl, and heterocycloalkyl are each substituted or unsubstituted.

Each R¹⁰ is independently H, (C₁-C₆)alkyl, (C₂-C₆)alkenyl, (C₂-C₆)alkynyl, (C₃-C₈)cycloalkyl, 5-10 membered heterocycloalkyl, aryl, aralkyl, heteroaryl, or heteroaralkyl; wherein said alkyl, alkenyl, alkynyl, alkoxy, cycloalkyl, heterocycloalkyl, aryl, aralkyl, heteroaryl, and heteroaralkyl are optionally and independently substituted with halogen, —CN, —OH, —NH₂, —NH(C₁-C₃)alkyl, —N((C₁-C₃)alkyl)₂, —CONH₂, —CONH(C₁-C₃)alkyl, —CON((C₁-C₃)alkyl)₂, —CO(C₁-C₃)alkyl, —CO₂H, (C₁-C₃)alkyl, (C₁-C₃)haloalkyl, (C₁-C₃)alkoxy, (C₁-C₃)haloalkyl, (C₁-C₃)haloalkoxy, (C₁-C₃)alkyoxycarbonyl, (C₃-C₇)cycloalkyl, or phenyl.

Also included within the scope of the invention are the compounds exmplified in Examples 1-42 and in Table I, and pharmaceutically acceptable salts thereof.

Additionally, the present invention provides a method of treating a subject in need of inhibition of a kinase protein comprising administering to a subject in need thereof an effective amount of a kinase inhibitor according to Formula I.

The present invention provides a method of reducing cancer metastatis in a subject with cancer comprising administering to a subject in need thereof an effective amount of a kinase inhibitor according to Formula I.

Further embodiments of the present invention include: a compound according to Formula I for use as a medicament; use of the compound according to Formula I for the preparation of a medicament for the treatment a subject in need of inhibition of a kinase protein; and use of the compound according to Formula I for the preparation of a medicament for the suppression (reduction) of cancer metastatis in a subject in need thereof.

The present invention also encompasses a compound according to Formula I, or a pharmaceutically acceptable salt thereof, for use in therapy. Additionally included is the use of a disclosed protein kinase inhibitor, according to Formula I, or a pharmaceutically acceptable salt thereof, for therapy, such as treating a subject in need of inhibition of a kinase protein, wherein the subject has a hyperproliferative disease or an inflammatory disease.

Additionally, the present invention includes a pharmaceutical composition comprising an effective amount of a compound according to Formula I and a pharmaceutically acceptable carrier, excipient or diluent.

### DETAILED DESCRIPTION OF THE INVENTION

One aspect of the present invention is to provide novel compounds according to Formula I that are useful in the treatment of hyperproliferative diseases and inflammatory diseases. Specifically, the compounds of the invention are protein kinase inhibitors. As a result, this invention provides in a first aspect novel compounds according to Formula I, as well as pharmaceutically acceptable salts and pharmaceutically active derivatives thereof, that are useful for the treatment of a subject in need of inhibition of a protein kinase. Values and particular values for the variables in Formula I are provided in the following paragraphs.

Ar is as described above. Alternatively, Ar is selected from the group consisting of phenyl, pyridinyl, pyrimidinyl, imidazolyl, 1H-indolyl, 2-oxo-indolinyl, benzo[1,3-d]dioxolyl and furanyl, each optionally and independently substituted with up to 3 substituents represented by R³. Another possibility is when Ar is optionally substituted phenyl. Alternatively, Ar is optionally substituted pyridinyl. Alternatively, Ar is optionally substituted pyrimidinyl. Further, Ar can be optionally substituted imidazolyl. Ar can also be optionally substituted 1H-indolinyl. Alternatively, Ar is optionally substituted 2-oxo-indolinyl. Another possibility is when Ar is optionally substituted benzo[1,3-d]dioxolyl. Ar can alternatively be optionally substituted furanyl.

R¹ and R² are as described above. Alternatively, R¹ and R² are independently selected from H, halogen, cyano, (C₁-C₆)alkyl, (C₂-C₆)alkenyl, (C₂-C₆)alkynyl, (C₁-C₆)alkoxy, (C₃-C₈)cycloalkyl, 5-10 membered heterocycloalkyl, —C(O)OR⁵, —C(O)R⁵, —OC(O)R⁵, —C(O)NR⁵R⁶, —NR⁵C(O)NR⁵R⁶, —NR⁵C(O)OR⁵, —NR⁵C(O)R⁵, —NR⁵C(S)NR⁵R⁶, —S(O)ₚNR⁵R⁶, —NR⁵R⁶, —S(O)ₚR⁵, —NR⁵S(O)ₚR⁵; or R¹ and R² can be taken together with their intervening atoms to form a (C₅-C₆)cycloalkyl ring; wherein said alkyl, alkenyl, alkynyl, alkoxy, cycloalkyl, heterocycloalkyl or (C₅-C₆)cycloalkyl ring represented by R¹ and/or R² are optionally and independently substituted with halogen, —CN, —OH, —NH₂, —NH(C₁-C₆)alkyl, —N((C₁-C₆)alkyl)₂, (C₁-C₆)alkoxy, (C₁-C₆)alkoxycarbonyl, —CONH₂, —OCONH₂, —NHCONH₂, —N(C₁-C₆)alkylCONH₂, —N(C₁-C₆)alkylCONH(C₁-C₆)alkyl, —NHCONH(C₁-C₆)alkyl, —NHCON((C₁-C₆)alkyl)₂, —N(C₁-C₆)alkylCON((C₁-C₆)alkyl)₂, —NHC(S)NH₂, —N(C₁-C₆)alkylC(S)NH₂, —N(C₁-C₆)alkylC(S)NH(C₁-C₆)alkyl, —NHC(S)NH(C₁-C₆)alkyl, —NHC(S)N((C₁-C₆)alkyl)₂, —N(C₁-C₆)alkylC(S)N((C₁-C₆)alkyl)₂, —CONH(C₁-C₆)alkyl, —OCONH(C₁-C₆)alkyl —CON((C₁-C₆)alkyl)₂, —C(S)(C₁-C₆)alkyl, —S(O)ₚ(C₁-C₆)alkyl, —S(O)ₚNH₂, —S(O)ₚNH(C₁-C₆)alkyl, —S(O)ₚN((C₁-C₆)alkyl)₂, —CO(C₁-C₆)alkyl, —OCO(C₁-C₆)alkyl, —C(O)O(C₁-C₆)alkyl, —OC(O)O(C₁-C₆)alkyl, —C(O)H or —CO₂H. Alternatively, each R¹ and R² is independently H or (C₁-C₆)alkyl, wherein the alkyl group is optionally substituted with halogen, —OH, —CN, —NH₂, (C₁-C₃)alkoxy, (C₁-C₃)haloalkoxy, phenyl, halophenyl, hydroxyphenyl, or methoxyphenyl.

R³ is as described above. Alternatively, each R³ is independently: i) halogen, —X₁—OH, —X₁—CN, —X₁—CO₂R¹⁰, —X₁—OR¹⁰, —X₁—NR¹⁰C(O)N(R¹⁰)₂, —X₁—NR¹⁰C(S)N(R¹⁰)₂, —X₁—COR¹⁰, —X₁—N(R¹⁰)₂, —X₁—N(R¹⁰)₃, —X₁—OCOR¹⁰, —X₁—SO₂N(R¹⁰)₂, —X₁—S(O)ₙR¹⁰, —X₁—NR¹⁰S(O)ₙR¹⁰, —X₁—NR¹⁰COR¹⁰, —X₁—CON(R¹⁰)₂, or —X₁—NR¹⁰CO₂R¹⁰; or

ii) (C₁-C₆)alkyl, (C₁-C₆)haloalkyl, (C₂-C₆)alkenyl, (C₂-C₆)haloalkenyl, (C₂-C₆)alkynyl or (C₂-C₆)haloalkynyl; or iii) phenyl, thienyl, oxazolyl, isooxazolyl, oxadiazolyl, thiadiazolyl, thiazolyl, pyridyl, pyrazolyl, or pyrrolyl, each optionally and independently substituted with up to 2 groups selected from halogen, —CN, —OH, —NH₂, (C₁-C₃)alkyl, halo(C₁-C₃)alkyl, phenyl[optionally substituted with halogen, (C₁-C₃)alkyl, (C₁-C₃)alkoxy, (C₁-C₃)haloalkyl, (C₁-C₃)haloalkoxy, —CN or —NO₂], (C₁-C₃)alkoxy, halo(C₁-C₃)alkoxy, —CO₂(C₁-C₃)alkyl, —CONH₂, —CONH(C₁-C₃)alkyl, —CO(C₁-C₃)alkyl or —CO₂H; or iv) 1,3-dioxolanyl, 1,3-dioxanyl, (C₃-C₆)cycloalkyl, piperidinyl or morpholinyl, each optionally and independently substituted with up to 2 groups selected from halogen, —OH, —NH₂, —O(C₁-C₃)alkyl, (C₁-C₃)alkyl, phenyl, —CO₂H, oxo and thioxo. In another alternative, each R³ is independently —F, —Cl, —CN, —COMe, —CONH₂, —CO₂Me, —CO(cyclopropyl), —OCF₃, —OMe, —O-iPr, —OCHF₂, —OCH₂CN, —NH₂, —NHCOMe, —NMe₂, —NHPh, -Me, -Et, allyl, -Ph, —CF₃, —CH₂CN, —CH₂OH, —CH₂CH₂OH, —CH(OH)CH₃, —CH₂COMe, —CH₂CO₂H, —CH₂NH₂, —CH₂NHCOCF₃, —SO₂NH₂, —SO₂Me, or a group selected from:

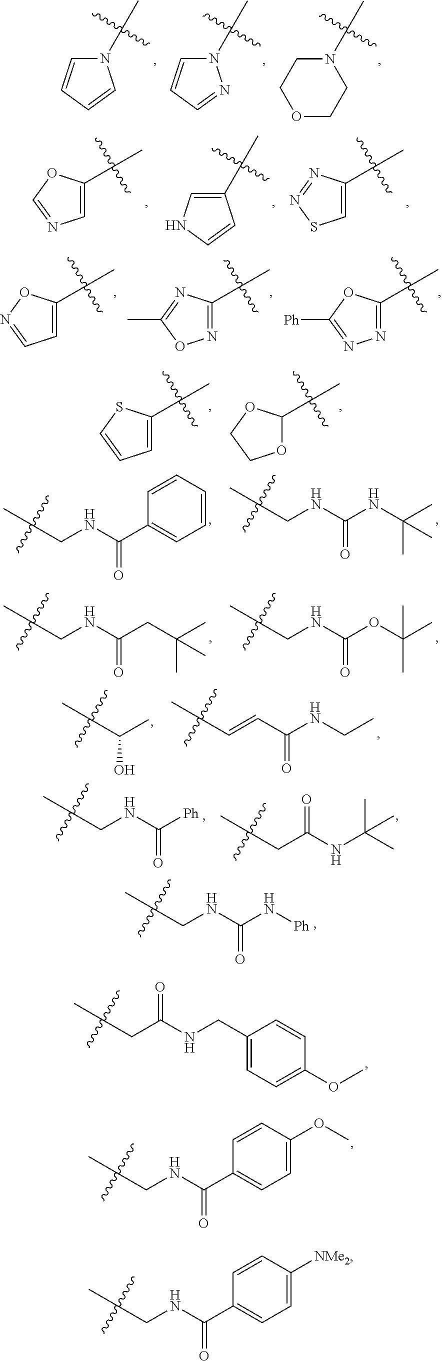

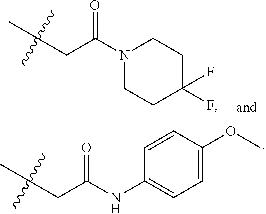

In another alternative, one group represented by R³ is represented by a structural formula selected from:

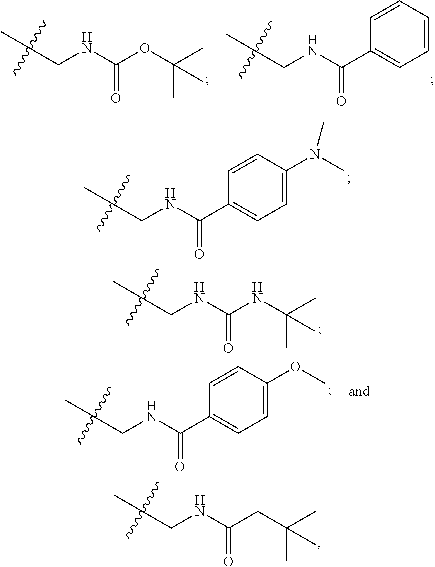

and the other R³ group(s), if present, are independently selected from halogen, (C₁-C₃)alkyl, (C₁-C₃)alkoxy, (C₁-C₃)haloalkyl, (C₁-C₃)haloalkoxy, —CN and —NO₂.In yet another alternative, each R³ is independently halogen, (C₁-C₆)alkyl, halo(C₁-C₆)alkyl, (C₁-C₆)alkoxy, —CN, —CO(C₁-C₄)alkyl, —CO₂(C₁-C₄)alkyl, —NH₂, —NH(C₁-C₆)alkyl, —N((C₁-C₆)alkyl)₂, —NHCO(C₁-C₆)alkyl, —CH₂NHCOCF₃,

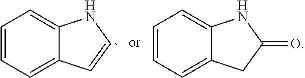

R⁴ is as described above. Alternatively, each R⁴ is independently halogen, —OH, —NH₂, —O(C₁-C₃)alkyl, (C₁-C₃)alkyl, phenyl, —CO₂H, oxo or thioxo.

R⁷ and R⁸ are as described above. Alternatively, R⁷ and R⁸ are independently H, halogen, cyano, (C₁-C₆)alkyl, (C₂-C₆)alkenyl, (C₂-C₆)alkynyl, —OR⁵, (C₃-C₈)cycloalkyl, 5-10 membered heterocycloalkyl, —C(O)OR⁵, —C(O)R⁵, —OC(O)R⁵, —C(O)NR⁵R⁶, —NR⁵C(O)NR⁵R⁶, —NR⁵C(O)OR⁵, —NR⁵C(O)R⁵, —NR⁵C(S)NR⁵R⁶, —S(O)ₚNR⁵R⁶, —NR⁵R⁶, —SR^{S}, —NR⁵S(O)ₚR⁵, wherein said alkyl, alkenyl, alkynyl, cycloalkyl, and heterocycloalkyl represented by R⁷ and R⁸ are each optionally substituted with halogen, —CN, —OH, —NH₂, —NH(C₁-C₆)alkyl, —N((C₁-C₆)alkyl)₂, (C₁-C₆)alkoxy, (C₁-C₆)alkoxycarbonyl, —CONH₂, —OCONH₂, —NHCONH₂, —N(C₁-C₆)alkylCONH₂, —N(C₁-C₆)alkylCONH(C₁-C₆)alkyl, —NHCONH(C₁-C₆)alkyl, —NHCON((C₁-C₆)alkyl)₂, —N(C₁-C₆)alkylCON((C₁-C₆)alkyl)₂, —NHC(S)NH₂, —N(C₁-C₆)alkylC(S)NH₂, —N(C₁-C₆)alkylC(S)NH(C₁-C₆)alkyl, —NHC(S)NH(C₁-C₆)alkyl, —NHC(S)N((C₁-C₆)alkyl)₂, —N(C₁-C₆)alkylC(S)(S)N((C₁-C₆)alkyl)₂, —CONH(C₁-C₆)alkyl, —OCONH(C₁-C₆)alkyl —CON((C₁-C₆)alkyl)₂, —C(S)(C₁-C₆)alkyl, —S(O)ₚ(C₁-C₆)alkyl, —S(O)ₚNH₂, —S(O)ₚNH(C₁-C₆)alkyl, —S(O)ₚN((C₁-C₆)alkyl)₂, —CO(C₁-C₆)alkyl, —OCO(C₁-C₆)alkyl, —C(O)O(C₁-C₆)alkyl, —OC(O)O(C₁-C₆)alkyl, —C(O)H or —CO₂H. In another alternative, R⁷ and R⁸ are independently H, halogen or (C₁-C₆)alkyl, wherein the alkyl is optionally substituted by halogen, —CN, —OH, —NH₂, —NH(C₁-C₆)alkyl, —N((C₁-C₆)alkyl)₂, (C₁-C₆)alkoxy, (C₁-C₆)alkoxycarbonyl, —CONH₂, —CONH(C₁-C₆)alkyl, —CON((C₁-C₆)alkyl)₂, —CO(C₁-C₆)alkyl or —CO₂H.

R¹⁰ is as described above. Alternatively, each R¹⁰ is independently H, (C₁-C₆)alkyl, (C₃-C₆)cycloalkyl, piperidinyl, morpholinyl, benzyl or phenyl; wherein the alkyl, cycloalkyl, piperidinyl, morpholinyl, benzyl and phenyl groups represented by R¹⁰ are optionally and independently substituted with halogen, —CN, —OH, —NH₂, —NH(C₁-C₃)alkyl, —N((C₁-C₃)alkyl)₂, —COMe, —CO₂H, (C₁-C₃)alkyl, halo(C₁-C₃)alkyl, (C₁-C₃)alkoxy or halo(C₁-C₃)alkoxy.

W, Y, X, p, n, R⁵ and R⁶ are all as described above.

Each X₁ is independently a covalent bond, a (C₁-C₆)alkylene, (C₁-C₆)alkenylene or (C₁-C₆)alkynylene. ALternatively, each X₁ is independently a covalent bond or a (C₁-C₂)alkylene.

In another aspect, the present invention provides a compound according to Formulae II, IIa-IIf, III, and IIIa-IIIi:

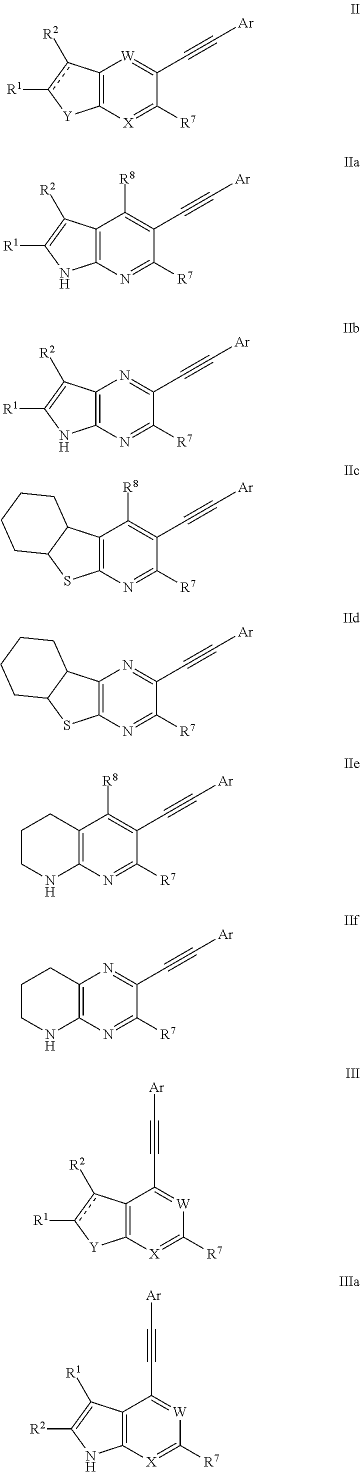

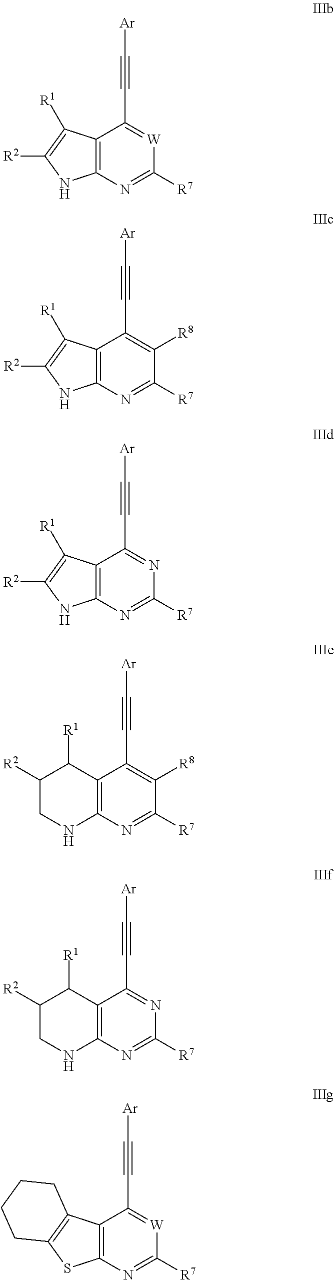

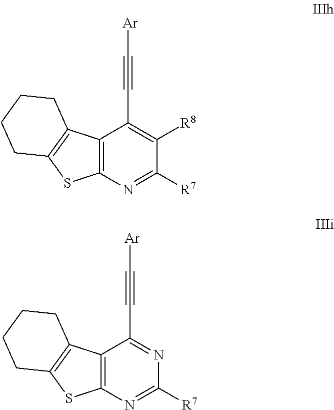

as well as pharmaceutically acceptable salts thereof, wherein the values and particular values for the variables, where present, are as described for Formula I.

In a first specific embodiment, the variables of Formulae I, II, IIa-IIf, III, and IIIa-IIIi have the following meanings:

R¹ and R², where present, are independently selected from H, halogen, cyano, (C₁-C₆)alkyl, (C₂-C₆)alkenyl, (C₂-C₆)alkynyl, (C₁-C₆)alkoxy, (C₃-C₈)cycloalkyl, 5-10 membered heterocycloalkyl, —C(O)OR⁵, —C(O)R⁵, —OC(O)R⁵, —C(O)NR⁵R⁶, —NR⁵C(O)NR⁵R⁶, —NR⁵C(O)OR⁵, —NR⁵C(O)R⁵, —NR⁵C(S)NR⁵R⁶, —S(O)ₚNR⁵R⁶, —NR⁵R⁶, —S(O)ₚR⁵, —NR⁵S(O)ₚR⁵; or R¹ and R² can be taken together with their intervening atoms to form a (Cₛ—C₆)cycloalkyl ring; wherein said alkyl, alkenyl, alkynyl, alkoxy, cycloalkyl, heterocycloalkyl or (Cₛ—C₆)cycloalkyl ring represented by R¹ and/or R² are optionally and independently substituted with halogen, —CN, —OH, —NH₂, —NH(C₁-C₆)alkyl, —N((C₁-C₆)alkyl)₂, (C₁-C₆)alkoxy, (C₁-C₆)alkoxycarbonyl, —CONH₂, —OCONH₂, —NHCONH₂, —N(C₁-C₆)alkylCONH₂, —N(C₁-C₆)alkylCONH(C₁-C₆)alkyl, —NHCONH(C₁-C₆)alkyl, —NHCON((C₁-C₆)alkyl)₂, —N(C₁-C₆)alkylCON((C₁-C₆)alkyl)₂, —NHC(S)NH₂, —N(C₁-C₆)alkylC(S)NH₂, —N(C₁-C₆)alkylC(S)NH(C₁-C₆)alkyl, —NHC(S)NH(C₁-C₆)alkyl, —NHC(S)N((C₁-C₆)alkyl)₂, —N(C₁-C₆)alkylC(S)N((C₁-C₆)alkyl)₂, —CONH(C₁-C₆)alkyl, —OCONH(C₁-C₆)alkyl —CON((C₁-C₆)alkyl)₂, —C(S)(C₁-C₆)alkyl, —S(O)ₚ(C₁-C₆)alkyl, —S(O)ₚNH₂, —S(O)ₚNH(C₁-C₆)alkyl, —S(O)ₚN((C₁-C₆)alkyl)₂, —CO(C₁-C₆)alkyl, —OCO(C₁-C₆)alkyl, —C(O)O(C₁-C₆)alkyl, —OC(O)O(C₁-C₆)alkyl, —C(O)H or —CO₂H.

R⁷ and R⁸, where present, are independently H, halogen, cyano, (C₁-C₆)alkyl, (C₂-C₆)alkenyl, (C₂-C₆)alkynyl, —OR⁵, (C₃-C₈)cycloalkyl, 5-10 membered heterocycloalkyl, —C(O)OR⁵, —C(O)R⁵, —OC(O)R⁵, —C(O)NR⁵R⁶, —NR⁵C(O)NR⁵R⁶, —NR⁵C(O)OR⁵, —NR⁵C(O)R⁵, —NR⁵C(S)NR⁵R⁶, —S(O)ₚNR⁵R⁶, —NR⁵R⁶, —SR⁵, —NR⁵S(O)ₚR⁵, wherein said alkyl, alkenyl, alkynyl, cycloalkyl, and heterocycloalkyl represented by R⁷ and R⁸ are each optionally substituted with halogen, —CN, —OH, —NH₂, —NH(C₁-C₆)alkyl, —N((C₁-C₆)alkyl)₂, (C₁-C₆)alkoxy, (C₁-C₆)alkoxycarbonyl, —CONH₂, —OCONH₂, —NHCONH₂, —N(C₁-C₆)alkylCONH₂, —N(C₁-C₆)alkylCONH(C₁-C₆)alkyl, —NHCONH(C₁-C₆)alkyl, —NHCON((C₁-C₆)alkyl)₂, —N(C₁-C₆)alkylCON((C₁-C₆)alkyl)₂, —NHC(S)NH₂, —N(C₁-C₆)alkylC(S)NH₂, —N(C₁-C₆)alkylC(S)NH(C₁-C₆)alkyl, —NHC(S)NH(C₁-C₆)alkyl, —NHC(S)NH(C₁-C₆)alkyl)₂, —N(C₁-C₆)alkylC(S)N((C₁-C₆)alkyl)₂, —CONH(C₁-C₆)alkyl, —OCONH(C₁-C₆)alkyl —CON((C₁-C₆)alkyl)₂, —C(S)(C₁-C₆)alkyl, —S(O)ₚ(C₁-C₆)alkyl, —S(O)ₚNH₂, —S(O)ₚNH(C₁-C₆)alkyl, —S(O)ₚN((C₁-C₆)alkyl)₂, —CO(C₁-C₆)alkyl, —OCO(C₁-C₆)alkyl, —C(O)O(C₁-C₆)alkyl, —OC(O)O(C₁-C₆)alkyl, —C(O)H or —CO₂H, and the rest of the variables have values and particular values as described for Formula I.

In a second specific embodiment, the variables of Formulae I, II, IIa-IIf, III, and IIIa-IIIi, Ar is selected from the group consisting of phenyl, pyridinyl, pyrimidinyl, imidazolyl, 1H-indolyl, 2-oxo-indolinyl, benzo[1,3-d]dioxolyl and furanyl, each optionally and independently substituted with up to 3 independently selected substituents represented by R³, and the rest of the values and particular values of the variables are as described for Formula I. Alternatively, R¹, R², R⁷ and R⁸ are as described in the first specific embodiment and the rest of the values and particular values of the variables are as described for Formula I.

In a third specific embodiment, the variables of Formulae I, II, IIa-IIf, III, and IIIa-IIIi, R¹ and R², where present, are each independently H or (C₁-C₆)alkyl, wherein the alkyl group is optionally substituted with halogen, —OH, —CN, —NH₂, (C₁-C₃)alkoxy, (C₁-C₃)haloalkoxy, or phenyl, and the rest of the values and particular values of the variables are as described for Formula I. Alternatively, Ar is as described for the second embodiment, and the rest of the values and particular values of the variables are as described for Formula I.

In a fourth specific embodiment, the variables of Formulae I, II, IIa-IIf, III, and IIIa-IIIi have the following meanings:

Each R³ is independently: i) halogen, —X₁—OH, —X₁—CN, —X₁—CO₂R¹⁰, —X₁—OR¹⁰, —X₁—NR¹⁰C(O)N(R¹⁰)₂.—X₁—NR¹⁰C(S)N(R¹⁰)₂, —X₁—COR¹⁰, —X₁—N(R¹⁰)₂, —X₁—N(R¹⁰)₃, —X₁—OCOR¹⁰, —X₁SO₂N(R¹⁰)₂, —X₁—S(O)ₙR¹⁰, —X₁—NR¹⁰S(O)ₙR¹⁰, —X₁—NR¹⁰COR¹⁰, —X₁—CON(R¹⁰)₂, or —X₁—NR¹⁰CO₂R¹⁰; or ii) (C₁-C₆)alkyl, (C₁-C₆)haloalkyl, (C₂-C₆)alkenyl, (C₂-C₆)haloalkenyl, (C₂-C₆)alkynyl or (C₂-C₆)haloalkynyl; or

iii) phenyl, thienyl, oxazolyl, isooxazolyl, oxadiazolyl, thiadiazolyl, thiazolyl, pyridyl, pyrazolyl, or pyrrolyl, each optionally and independently substituted with up to 2 groups selected from halogen, —CN, —OH, —NH₂, (C₁-C₃)alkyl, halo(C₁-C₃)alkyl, phenyl[optionally substituted with halogen, (C₁-C₃)alkyl, (C₁-C₃)alkoxy, (C₁-C₃)haloalkyl, (C₁-C₃)haloalkoxy, —CN or —NO₂], (C₁-C₃)alkoxy, halo(C₁-C₃)alkoxy, —CO₂(C₁-C₃)alkyl, —CONH₂, —CONH(C₁-C₃)alkyl, —CO(C₁-C₃)alkyl or —CO₂H; or
iv) 1,3-dioxolanyl, 1,3-dioxanyl, (C₃-C₆)cycloalkyl, piperidinyl or morpholinyl, each optionally and independently substituted with up to 2 groups selected from halogen, —OH, —NH₂, —O(C₁-C₃)alkyl, (C₁-C₃)alkyl, phenyl, —CO₂H, oxo and thioxo

Each R⁴ is independently halogen, —OH, —NH₂, —O(C₁-C₃)alkyl, (C₁-C₃)alkyl, phenyl, —CO₂H, oxo or thioxo.

Each X₁ is independently a covalent bond or (C₁-C₂)alkylene.

The variable n is an integer from 0 to 2.

R⁷ and R⁸ are independently H, halogen or (C₁-C₆)alkyl, wherein the alkyl is optionally substituted by halogen, —CN, —OH, —NH₂, —NH(C₁-C₆)alkyl, —N((C₁-C₆)alkyl)₂, (C₁-C₆)alkoxy, (C₁-C₆)alkoxycarbonyl, —CONH₂, —CONH(C₁-C₆)alkyl, —CON((C₁-C₆)alkyl)₂, —CO(C₁-C₆)alkyl or —CO₂H.

each R¹⁰ is independently H, (C₁-C₆)alkyl, (C₃-C₆)cycloalkyl, piperidinyl, morpholinyl, benzyl or phenyl; wherein the alkyl, cycloalkyl, piperidinyl, morpholinyl, benzyl and phenyl groups represented by R¹⁰ are optionally and independently substituted with halogen, —CN, —OH, —NH₂, —NH(C₁-C₃)alkyl, —N((C₁-C₃)alkyl)₂, —COMe, —CO₂H, (C₁-C₃)alkyl, halo(C₁-C₃)alkyl, (C₁-C₃)alkoxy or halo(C₁-C₃)alkoxy. wherein the values and particular values of the rest of the variables are as described for Formula I. Alternatively, Ar is as described in the second embodiment, and the values and particular values of the rest of the variables are as described for Formula I.

In a fifth specific embodiment, the variables of Formulae I, II, IIa-IIf, III, and IIIa-IIIi have the following meanings:

Ar is a phenyl, optionally substituted with up to 3 substituents, R³, and each R³ is independently —F, —Cl, —CN, —COMe, —CONH₂, —CO₂Me, —CO(cyclopropyl), —OCF₃, —OMe, —O-iPr, —OCHF₂, —OCH₂CN, —NH₂, —NHCOMe, —NMe₂, —NHPh, -Me, -Et, allyl, -Ph, —CF₃, —CH₂CN, —CH₂OH, —CH₂CH₂OH, —CH(OH)CH₃, —CH₂COMe, —CH₂CO₂H, —CH₂NH₂, —CH₂NHCOCF₃, —SO₂NH₂, —SO₂Me, or a group selected from:

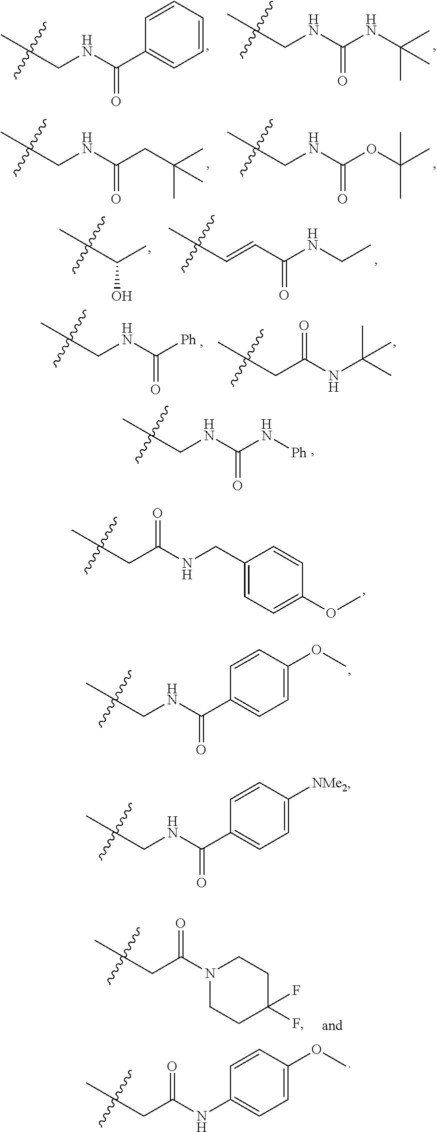

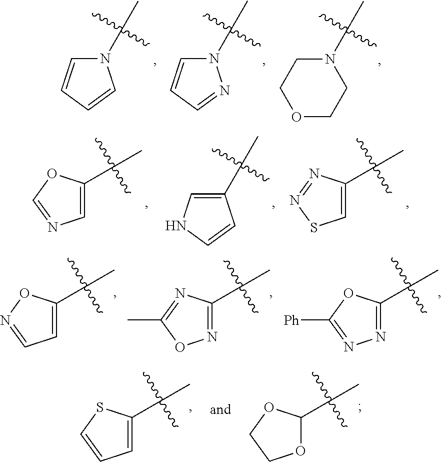

or Ar is a phenyl, optionally substituted with 1, 2 or 3 substituents, R³, wherein on substituent represented by R³ is represented by a structural formula selected from:

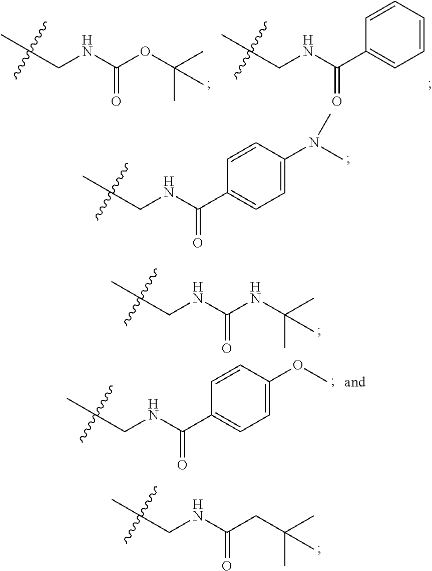

and the other R³ group(s), if present, are selected from halogen, (C₁-C₃)alkyl, (C₁-C₃)alkoxy, (C₁-C₃)haloalkyl, (C₁-C₃)haloalkoxy, —CN and —NO₂, and wherein the values and particular values of the rest of the variables are as described for Formula I. Alternatively, R⁴, n, R⁷, R⁸ and R¹⁰ are as described in the fourth embodiment, and the values and particular values of the rest of the variables are as described for Formula I.

In a sixth specific embodiment, the variables of Formulae I, II, IIa-IIf, III, and IIIa-IIIi have the following meanings:

Ar is phenyl, pyridinyl, 1H-indolyl, 2-oxo-indolinyl, pyrimidinyl, benzo[1,3-d]dioxolyl or furanyl, each optionally substituted with up to 3 substituents, R³.

And, each R³ is independently

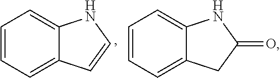

halogen, (C₁-C₆)alkyl, halo(C₁-C₆)alkyl, (C₁-C₆)alkoxy, —CN, —CO(C₁-C₄)alkyl, —CO₂(C₁-C₄)alkyl, —NH₂, —NH(C₁-C₆)alkyl, —N((C₁-C₆)alkyl)₂, —NHCO(C₁-C₆)alkyl, or —CH₂NHCOCF₃, wherein the values and particular values of the rest of the variables are as described for Formula I. Alternatively, R⁴, n, R⁷, R⁸ and R¹⁰ are as described in the fourth embodiment, and the values and particular values of the rest of the variables are as described for Formula I.

In a seventh specific embodiment, the variables of Formulae I, II, IIa-IIf, III, and IIIa-IIIi have the following meanings:

Ar is phenyl, pyridinyl, 1H-indolyl, 2-oxo-indolinyl, pyrimidinyl, benzo[1,3-d]dioxolyl or furanyl, each optionally substituted with up to 3 substituents, R³.

And, each R³ is independently

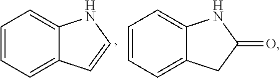

halogen, (C₁-C₆)alkyl, halo(C₁-C₆)alkyl, (C₁-C₆)alkoxy, —CN, —CO(C₁-C₄)alkyl, —CO₂(C₁-C₄)alkyl, —NH₂, —NH(C₁-C₆)alkyl, —N((C₁-C₆)alkyl)₂, —NHCO(C₁-C₆)alkyl, or —CH₂NHCOCF₃, provided that one R³ is represented by a structural formula selected from:

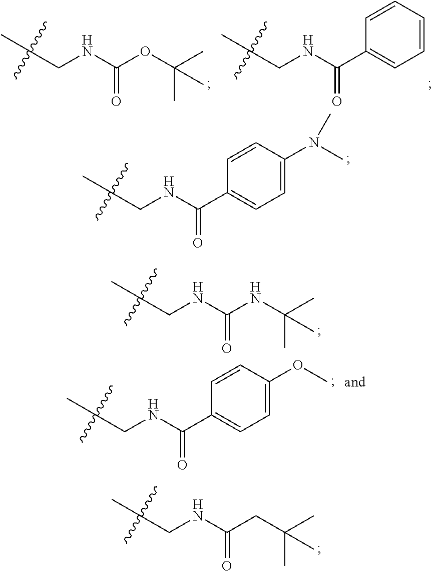

wherein the values and particular values of the rest of the variables are as described for Formula I. Alternatively, R⁴, n, R⁷, R⁸ and R¹⁰ are as described in the fourth embodiment, and the values and particular values of the rest of the variables are as described for Formula I.

A further embodiment of the invention, a compound according to Formulae I, II, IIa-IIf, III or IIIa-IIIi excludes the two following compounds (group A), and pharmaceutically acceptable salts thereof:

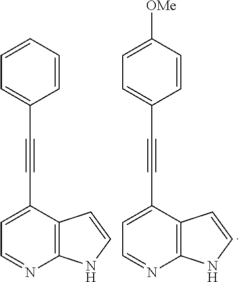

An alternative embodiment of the invention is a compound according to Formulae I, II, IIa-IIf, III or IIIa-IIIi that excludes compounds (group B), and pharmaceutically acceptable salts thereof, wherein X and W are N; Y is S; R¹ and R² are taken together with their intervening atoms to form an unsubstituted cyclohexyl, there is a double bond between R¹ and R²; R⁷ is H; and Ar is phenyl substituted with halogen, methyl or CF₃, and additionally substituted with 0-4 groups selected from halogen, OH, CN, CF₃, NO₂, (C₁-C₄)alkyl, (C₁-C₄)alkenyl, and (C₁-C₄)alkynyl.

Alternatively, the present invention excludes compounds according to Formulae I, II, IIa-IIf, III or IIIa-IIIi wherein Ar is a substituted or unsubstituted 2,4-diamino-1,3-pyrimidinyl (group C), and pharmaceutically acceptable salts thereof.

Additionally, the present invention embodies compounds according to Formulae I, II, IIa-IIf, III or IIIa-IIIi which exclude compounds from group A and group B; or compounds according to Formulae I, II, IIa-IIf, III or IIIa-IIIi which exclude compounds from group A and group C; or compounds according to Formulae I, II, IIa-IIf, III or IIIa-IIIi which exclude compounds from group B and group C; or compounds according to Formulae I, II, IIa-IIf, III or IIIa-IIIi which exclude compounds from groups A, B and C.

### DEFINITIONS

“Cₘ-Cₙ,” or “Cₘ-Cₙ group” used alone or as a prefix, refers to any group having m to n carbon atoms.

“Cycloalkyl” refers to a monocyclic or polycyclic, non-aromatic ring system of 3 to 20 carbon atoms, 3 to 12 carbon atoms, or 3 to 8 carbon atoms, which may be saturated or unsaturated. Examples of cycloalkyl groups include cyclopropyl, cyclobutyl, cyclopentyl, cyclohexyl, cyclohexenyl, cyclohexa-1,3-dienyl, cyclooctyl, cycloheptanyl, norbornyl, adamantyl, and the like.

“Heterocycloalkyl” refers to a saturated or unsaturated, non-aromatic, monocyclic or polycyclic ring system of 3 to 20 atoms, 3 to 12 atoms, or 3 to 8 atoms, containing one to four ring heteroatoms chosen from O, N and S. Examples of heterocycloalkyl groups include pyrrolidine, piperidine, tetrahydrofuran, tetrahydropyran, tetrahydrothiophene, tetrahydrothiopyran, isoxazolidine, 1,3-dioxolane, 1,3-dithiolane, 1,3-dioxane, 1,4-dioxane, 1,3-dithiane, 1,4-dithiane, morpholine, thiomorpholine, thiomorpholine-1,1-dioxide, tetrahydro-2H-1,2-thiazine-1,1-dioxide, isothiazolidine-1,1-dioxide, pyrrolidin-2-one, piperidin-2-one, piperazin-2-one, and morpholin-2-one, and the like.

“Halogen” and “halo” refer to fluoro, chloro, bromo or iodo.

“Haloalkyl” refers to an alkyl group substituted with one or more halogen atoms. By analogy, “haloalkenyl”, “haloalkynyl”, etc., refers to the group (for example alkenyl or alkynyl) substituted by one or more halogen atomes.

“Cyano” refers to the group —CN.

“Oxo” refers to a divalent ═O group.

“Thioxo” refers to a divalent ═S group.

“Ph” refers to a phenyl group.

“Carbonyl” refers to a divalent —C(O)— group.

“Alkyl” refers to a straight or branched, saturated aliphatic group typically having 1 to 12 carbon atoms. More particularly, the aliphatic group may have 1 to 8, 1 to 6, or 1 to 4 carbon atoms. This term is exemplified by groups such as methyl, ethyl, n-propyl, isopropyl, n-butyl, isobutyl, tert-butyl, n-hexyl, and the like.

“Alkenyl” refers to a straight or branched aliphatic group with at least one double bond. Typically, alkenyl groups have from 2 to 12 carbon atoms, from 2 to 8, from 2 to 6, or from 2 to 4 carbon atoms. Examples of alkenyl groups include ethenyl (—CH═CH₂), n-2-propenyl (allyl, —CH₂CH═CH₂), pentenyl, hexenyl, and the like.

“Alkynyl” refers to a straight or branched aliphatic group having at least 1 site of alkynyl unsaturation. Typically, alkynyl groups contain 2 to 12, 2 to 8, 2 to 6 or 2 to 4 carbon atoms. Examples of alkynyl groups include ethynyl (—≡CH), propargyl (—CH₂≡CH), pentynyl, hexynyl, and the like.

“Alkylene” refers to a bivalent saturated straight-chained hydrocarbon, e.g., C₁-C₆ alkylene includes —(CH₂)₆—, —CH₂—CH—(CH₂)₃CH₃,

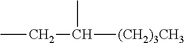

and the like. “Bivalent means that the alkylene group is attached to the remainder of the molecule through two different carbon atoms.

“Alkenylene” refers to an alkylene group with in which one carbon-carbon single bond is replaced with a double bond.

“Alkynylene” refers to an alkylene group with in which one carbon-carbon single bond is replaced with a triple bond.

“Aryl” refers to an aromatic carbocyclic group of from 6 to 14 carbon atoms having a single ring or multiple condensed rings. The term “aryl” also includes aromatic carbocycle(s) fused to cycloalkyl or heterocycloalkyl groups. Examples of aryl groups include phenyl, benzo[d][1,3]dioxole, naphthyl, phenantrenyl, and the like.

“Aryloxy” refers to an —OAr group, wherein 0 is an oxygen atom and Ar is an aryl group as defined above.

“Aralkyl” refers to an alkyl having at least one alkyl hydrogen atom replaced with an aryl moiety, such as benzyl, —(CH₂)₂-phenyl, —(CH₂)₃-phenyl, —CH(phenyl)₂, and the like.

“Alkyl cycloalkyl” refers to an alkyl having at least one alkyl hydrogen atom replaced with a cycloalkyl moiety, such as —CH₂-cyclohexyl, —CH₂-cyclohexenyl, and the like.

“Heteroaryl” refers to a 5 to 14 membered monocyclic, bicyclic or tricyclic heteroaromatic ring system, containing one to four ring heteroatoms selected from nitrogen, oxygen and sulfur. The term “heteroaryl” also includes heteroaromatic ring(s) fused to cycloalkyl or heterocycloalkyl groups. Particular examples of heteroaryl groups include optionally substituted pyridyl, pyrrolyl, pyrimidinyl, furyl, thienyl, imidazolyl, oxazolyl, isoxazolyl, thiazolyl, isothiazolyl, pyrazolyl, 1,2,3-triazolyl, 1,2,4-triazolyl, 1,2,3-oxadiazolyl, 1,2,4-oxadiazolyl, 1,2,5-oxadiazolyl, 1,3,4-oxadiazolyl,1,3,4-triazinyl, 1,2,3-triazinyl, benzofuryl, [2,3-dihydro]benzofuryl, isobenzofuryl, benzothienyl, benzotriazolyl, isobenzothienyl, indolyl, isoindolyl, 3H-indolyl, benzimidazolyl, imidazo[1,2-a]pyridyl, benzothiazolyl, benzoxa-zolyl, quinolizinyl, quinazolinyl, pthalazinyl, quinoxalinyl, cinnolinyl, napthyridinyl, pyrido[3,4-b]pyridyl, pyrido[3,2-b]pyridyl, pyrido[4,3-b]pyridyl, quinolyl, isoquinolyl, tetrazolyl, 1,2,3,4-tetrahydroquinolyl, 1,2,3,4-tetrahydroisoquinolyl, purinyl, pteridinyl, carbazolyl, xanthenyl, benzoquinolyl, and the like.

“Heteroaryloxy” refers to an -OHet group, wherein 0 is an oxygen atom and Het is a heteroaryl group as defined above.

“Heteroaralkyl” refers to an alkyl having at least one alkyl hydrogen atom replaced with a heteroaryl moiety, such as —CH₂-pyridinyl, —CH₂-pyrimidinyl, and the like.

“Alkoxy” refers to the group —O—R where R is “alkyl”, “cycloalkyl”, “alkenyl”, or “alkynyl”. Examples of alkoxy groups include for example, methoxy, ethoxy, ethenoxy, and the like.

“Alkyl heterocycloalkyl” refers to an alkyl having at least one alkyl hydrogen atom replaced with a heterocycloalkyl moiety, such as —CH₂-morpholino, —CH₂-piperidyl and the like.

“Alkoxycarbonyl” refers to the group —C(O)OR where R is “alkyl”, “alkenyl”, “alkynyl”, “cycloalkyl”, “heterocycloalkyl”, “aryl”, or “heteroaryl”.

Suitable substituents for “alkyl”, “alkenyl”, “alkynyl”, “cycloalkyl”, “heterocycloalkyl”, “aryl”, or “heteroaryl”, etc., are those which do not significantly affect protein kinase inhibitory activity, e.g., reduce activity by more than 10× or 100×. Examples of suitable substituents are those selected from the group consisting of halogen, —CN, —OH, —NH₂, (C₁-C₄)alkyl, (C₁-C₄)haloalkyl, aryl, heteroaryl, (C₃-C₇)cycloalkyl, (5-7 membered) heterocycloalkyl, —NH(C₁-C₆)alkyl, —N((C₁-C₆)alkyl)₂, (C₁-C₆)alkoxy, (C₁-C₆)alkoxycarbonyl, —CONH₂, —OCONH₂, —NHCONH₂, —N(C₁-C₆)alkylCONH₂, —N(C₁-C₆)alkylCONH(C₁-C₆)alkyl, —NHCONH(C₁-C₆)alkyl, —NHCON((C₁-C₆)alkyl)₂, —N(C₁-C₆)alkylCON((C₁-C₆)alkyl)₂, —NHC(S)NH₂, —N(C₁-C₆)alkylC(S)NH₂, —N(C₁-C₆)alkylC(S)NH(C₁-C₆)alkyl, —NHC(S)NH(C₁-C₆)alkyl, —NHC(S)N((C₁-C₆)alkyl)₂, —N(C₁-C₆)alkylC(S)N((C₁-C₆)alkyl)₂, —CONH(C₁-C₆)alkyl, —OCONH(C₁-C₆)alkyl —CON((C₁-C₆)alkyl)₂, —C(S)(C₁-C₆)alkyl, —S(O)ₚ(C₁-C₆)alkyl, —S(O)ₚNH₂, —S(O)ₚNH(C₁-C₆)alkyl, —S(O)ₚN((C₁-C₆)alkyl)₂, —CO(C₁-C₆)alkyl, —OCO(C₁-C₆)alkyl, —C(O)O(C₁-C₆)alkyl, —OC(O)O(C₁-C₆)alkyl, —C(O)H or —CO₂H. More particularly, the substituents are selected from halogen, —CN, —OH, —NH₂, (C₁-C₄)alkyl, (C₁-C₄)haloalkyl, (C₁-C₄)alkoxy, phenyl, and (C₃-C₇)cycloalkyl. Within the framework of this invention, said “substitution” is also meant to encompass situations where neighboring substituents undergo ring closure, in particular when vicinal functional substituents are involved, thus forming, e.g., lactams, lactones, cyclic anhydrides, acetals, thioacetals, aminals, and the like.

Where a compounds of the present invention has more than one conformation, i.e., a tautomer of an individual compound, both structures are claimed individually and together as mixtures in any ratio. An example of a functional group that commonly tautomerizes is a ketone ←→. Additionally, when a compound of the present invention is depicted as a stereoisomer, enantiomer, cis/trans isomer, and/or conformer, all isomers of the structure are claimed individually and as a mixture in any ratio (e.g., a racemic mixture of enantiomers).

As used herein the term “subject” typically means a human, but can also be an animal in need of treatment, e.g., companion animals (dogs, cats, and the like), farm animals (cows, pigs, horses, sheep, goats, and the like) and laboratory animals (rats, mice, guinea pigs, and the like).

“Treatment” and “treating” encompass reducing the symptoms, reducing the progression, postponing the onset and/or increasing the longevity of a subject in need of the inhibition of a protein kinase. The terms “treatment” and “treating” also encompass the prophylactic administration of a compound of the invention to a subject susceptible to a disease requiring inhibition of a protein kinase. Prophylactic treatment includes suppression (partially or completely) of said disease associated with the expression of protein kinases, and further includes reducing the severity of the disease if onset occurs. Prophylactic treatment additionally includes the administration of a protein kinase inhibitor of the invention before the onset of said disease, and can result in the delay of disease onset, and/or reducing the likelihood of developing the disease.

“Reducing cancer metastasis” refers to reducing the progression and/or delaying the onset of metastasis of cancer in a subject in need thereof. Alternatively, “reducing cancer metastasis” includes reducing the number of organs and/or systems affected by metastasis, and reduction of tumor growth and/or progression in the organs and/or systems affected by metastasis. “Reducing cancer metastasis” alternatively includes the prophylactic treatment of a patient with cancer who is at risk for metastasis in order to delay onset, avert onset, reduce the likelihood of onset, or reduce the severity of metastasis. Prophylactic treatment is particularly advantageous for administration to subjects with cancer who are at risk for cancer metastasis. The present invention includes administration of a protein kinase inhibitor according to Formula I after the primary tumor(s) is treated, or during treatment of the primary tumor. Compounds of the invention can be used to treat the primary tumor and/or in combination with an alternative method of treatment of the primary tumor (i.e., radiation therapy, surgery, etc.) to reduce cancer metastasis.

“A subject in need to inhibition of a kinase protein”, as used herein, refers, for example, to a subject with a hyperproliferative disease or an inflammatory disease that is associated with the expression or activity of protein kinases, either directly or indirectly. For example, many tumors produce protein kinases directly, which promotes tumor cell proliferation and/or tumor cell survival. Analogously, many tumors produce ligands that stimulate protein kinase production, and thus indirectly produce protein kinases, which in turn promotes tumor cell proliferation and/or survival. In many cases, the increased expression of the protein kinases is localized and not systematic. Thus, the present invention encompasses both the localized and systematic inhibition of protein kinases associated with hyperproliferative and inflammatory diseases.

“Protein kinase inhibitor” refers to a compound of the invention that binds to the protein kinase and thereby decreases its activity. Protein kinases that are within the scope of this invention include Akt, Axl, Aurora A, Aurora B, dyrk2, epha2, fgfr3, igflr, IKK2, JNK3, VEGFR1, VEGFR2, VEGFR3 (also known as Flt-4), KDR, MEK1, MET, P70s6K, Plk1, RSK1, Src, TrkA, Zap70, cKit, bRaf, EGFR, Jak2, PI3K, NPM-Alk, c-Abl, BTK, FAK, PDGFR, TAK1, LimK, Flt-3, PDK1 and Erk. In one embodiment, the protein kinase is VEGFR2, VEGFR3, PDK1 and/or Flt-3.

As used herein, the term “hyperproliferative disease” refers to a disease or medical condition involving pathological growth of cells. Hyperproliferative disease includes neoplasia such as cancer and cancer metastasis, including, but not limited to: carcinoma such as cancer of the bladder, breast, colon, kidney, liver, lung (including small cell lung cancer), esophagus, gall-bladder, ovary, pancreas, stomach, cervix, thyroid, prostate, and skin (including squamous cell carcinoma); hematopoietic tumors of lymphoid lineage (including leukemia, acute lympocytic leukemia, acute lymphoblastic leukemia, B-cell lymphoma, T-cell lymphoma, Hodgkin's lymphoma, non-Hodgkin's lymphoma, hairy cell lymphoma and Burkett's lymphoma); hematopoietic tumors of myeloid lineage (including acute and chronic myelogenous leukemias, myelodysplastic syndrome and promyelocytic leukemia); tumors of mesenchymal origin (including fibrosarcoma and rhabdomyosarcorma, and other sarcomas, e.g. soft tissue and bone); tumors of the central and peripheral nervous system (including astrocytoma, neuroblastoma, glioma and schwannomas); and other tumors, including melanoma, seminoma, teratocarcinoma, osteosarcoma, xenoderoma pigmentosum, keratoctanthoma, thyroid follicular cancer and Kaposi's sarcoma. Alternatively, the compounds of the invention are useful for the treatment of neoplasia selected from lung, colon, head and neck, prostate, leukemias, lymphomas and Kaposi's sarcoma.

Non-cancerous proliferative disorders include smooth muscle cell proliferation, systemic sclerosis, cirrhosis of the liver, adult respiratory distress syndrome, idiopathic cardiomyopathy, lupus erythematosus, retinopathy, e.g., diabetic retinopathy or other retinopathies, cardiac hyperplasia, reproductive system associated disorders such as benign prostatic hyperplasia and ovarian cysts, pulmonary fibrosis, endometriosis, fibromatosis, harmatomas, lymphangiomatosis, sarcoidosis, desmoid tumors and the like. Non-cancerous proliferative disorders also include hyperproliferation of cells in the skin such as psoriasis and its varied clinical forms, Reiter's syndrome, pityriasis rubra pilaris, and hyperproliferative variants of disorders of keratinization (e.g., actinic keratosis, senile keratosis), scleroderma, and the like.

Smooth muscle cell proliferation includes proliferative vascular disorders, for example, intimal smooth muscle cell hyperplasia, restenosis and vascular occlusion, particularly stenosis following biologically- or mechanically-mediated vascular injury, e.g., vascular injury associated with balloon angioplasty or vascular stenosis. Moreover, intimal smooth muscle cell hyperplasia can include hyperplasia in smooth muscle other than the vasculature, e.g., hyperplasia in bile duct blockage, in bronchial airways of the lung in asthma patients, in the kidneys of patients with renal interstitial fibrosis, and the like.

“Inflammatory disease” as used herein refers to a disease or condition characterized by inflammation. Inflammation encompasses the first response of the immune system to infection or irritation, and is sometimes referred to as the innate cascade. Inflammation typically is characterized by one or more of the following symptoms: redness, heat, swelling, pain, and dysfunction of the organs involved.

Examples of inflammatory diseases treatable as described herein include, without limitation, transplant rejection; chronic inflammatory disorders of the joints, such as arthritis, rheumatoid arthritis, juvenile idiopathic arthritis, ankylosing spondylitis, psoriatic arthritis, osteoarthritis and bone diseases associated with increased bone resorption; inflammatory bowel diseases, such as ileitis, ulcerative colitis, Barrett's syndrome, and Crohn's disease; inflammatory lung disorders, such as asthma, adult respiratory distress syndrome (ARDS), chronic obstructive pulmonary disease (COPD) or chronic obstructive airway disease; inflammatory disorders of the eye, such as corneal dystrophy, trachoma, onchocerciasis, uveitis, sympathetic ophthalmitis and endophthalmitis; chronic inflammatory disorders of the gum, such as gingivitis and periodontitis; tuberculosis; leprosy; inflammatory diseases of the kidney, such as uremic complications, glomerulonephritis and nephrosis; inflammatory diseases of the liver, such as viral hepatitis and autoimmune hepatitis; inflammatory disorders of the skin, such as sclerodermatitis, psoriasis, erythema, eczema, or contact dermatitis; inflammatory diseases of the central nervous system, such as stroke, chronic demyelinating diseases of the nervous system, multiple sclerosis, AIDS-related neurodegeneration and Alzheimer's disease, infectious meningitis, encephalomyelitis, Parkinson's disease, Huntington's disease, amyotrophic lateral sclerosis and viral or autoimmune encephalitis; autoimmune diseases, such as diabetes mellitus, immune-complex vasculitis, systemic lupus erythematosus (SLE); inflammatory diseases of the heart, such as cardiomyopathy, ischemic heart disease, hypercholesterolemia, and atherosclerosis; as well as inflammation resulting from various diseases such as preeclampsia, chronic liver failure, brain and spinal cord trauma, and cancer. Inflammatory diseases treatable as described herein further include systemic inflammations of the body. Examples of systemic inflammation include but are not limited to gram-positive or gram negative shock, sepsis, septic shock, hemorrhagic or anaphylactic shock, and systemic inflammatory response syndrome. Further examples of inflammatory disease include circulatory shock, hemorrhagic shock and cardiogenic shock.

More particularly, inflammatory diseases treatable as described herein include inflammatory rheumatoid or rheumatic disease, especially of manifestations at the locomotor apparatus, such as rheumatoid arthritis, juvenile arthritis or psoriasis arthropathy; paraneoplastic syndrome or tumor-induced inflammatory diseases; turbin effusion; collagenosis, such as systemic Lupus erythmatosus, poly-myositis, dermato-myositis; systemic sclerodermia or mixed collagenosis; post-infectious arthritis (where no living pathogenic organism can be found at or in the infected part of the body); seronegative spondylarthritis, such as spondylitis ankylosans; and vasculitis.

The compounds of the present invention are also useful for the treatment of inflammatory conditions associated with the deleterious effects of the VEGFs. Such conditions include ophthalmologic conditions such as corneal graft rejection, ocular neovascularization, retinal neovascularization including neovascularization following injury or infection, diabetic retinopathy, retrolental fibroplasias and neovascular glaucoma. Particularly, the compounds are useful to treat corneal graft rejection.

When a disclosed compound or its pharmaceutically acceptable salt is named or depicted by structure, it is to be understood that solvates or hydrates of the compound are also included. “Solvates” refer to crystalline forms wherein solvent molecules are incorporated into the crystal lattice during crystallization. Solvate may include water or nonaqueous solvents such as ethanol, isopropanol, DMSO, acetic acid, ethanolamine, and EtOAc. Solvates, wherein water is the solvent molecule incorporated into the crystal lattice, are typically referred to as “hydrates.” Hydrates include stoichiometric hydrates as well as compositions containing variable amounts of water.

While the compounds of the invention can be administered as the sole active pharmaceutical agent, they can also be used in combination with one or more compounds of the invention or other agents. When administered as a combination, the therapeutic agents can be formulated as separate compositions that are administered at the same time or sequentially at different times, or the therapeutic agents can be given as a single composition.

The phrase “co-therapy” or “combination-therapy”, refers to the a use of a compound the invention in conjunction with another pharmaceutical agent, and is intended to embrace administration of each agent in a sequential manner in a regimen that will provide beneficial effects of the drug combination. Additionally, it is intended to also embrace co-administration of these agents in a substantially simultaneous manner, such as in a single capsule having a fixed ratio of the active agents or in multiple, separate capsules for each agent.

Specifically, the administration of compounds of the present invention may be in conjunction with additional therapies known to those skilled in the art in the prevention or treatment of neoplasia, such as radiation therapy, or with cytostatic or cytotoxic agents.

If formulated as a fixed dose, such combination products employ the compounds of this invention within the accepted dosage ranges. Compounds of Formulae I, II, IIa-IIf, III, or IIIa-IIIi may also be administered sequentially with known anticancer or cytotoxic agents when a combination formulation is inappropriate. The invention is not limited in the sequence of administration; compounds of the invention may be administered either prior to, simultaneous with or after administration of the known anticancer or cytotoxic agent.

Currently, standard treatment of primary tumors consists of surgical excision followed by either radiation or IV administered chemotherapy. The typical chemotherapy regime consists of either DNA alkylating agents, DNA intercalating agents, CDK inhibitors, or microtubule poisons. The chemotherapy doses used are just below the maximal tolerated does and therefore dose limiting toxicities typically include, nausea, vomiting, diarrhea, hair loss, neutropenia and the like.

The invention also relates to the treatment of a hyperproliferative disease that comprises administering a therapeutically effective amount of a compound of Formulae I, II, IIa-IIf, III, or IIIa-IIIi, in combination with an anti-tumour agent selected from the group consisting of mitotic inhibitors, alkylating agents, antimetabolites, intercalating antibiotics, growth factor inhibitors, cell cycle inhibitors, enzyme inhibitors, topoisomerase inhibitors, biological response modifiers, antihormones, angiogenesis inhibitors, and anti-androgens. It is well known in the art which anti-tumour agents are effective in combination therapy.

“Pharmaceutically acceptable salts” refer to salts or complexes of the below-specified compounds of Formulae I, II, IIa-IIf, III, or IIIa-IIIi. Examples of such salts include, but are not limited to, base addition salts formed by reaction of compounds of Formulae I, II, IIa-IIf, III, or IIIa-IIIg with organic or inorganic bases such as hydroxide, carbonate or bicarbonate of a metal cation such as those selected in the group consisting of alkali metals (sodium, potassium or lithium), alkaline earth metals (e.g. calcium or magnesium), or with an organic primary, secondary or tertiary alkyl amine Amine salts derived from methylamine, dimethylamine, trimethylamine, ethylamine, diethylamine, triethylamine, morpholine, N-Me-D-glucamine, N,N′-bis(phenylmethyl)-1,2-ethanediamine, tromethamine, ethanolamine, diethanolamine, ethylenediamine, N-methylmorpholine, procaine, piperidine, piperazine and the like are contemplated being within the scope of the instant invention. Additional examples are salts which are formed with inorganic acids (e.g. hydrochloric acid, hydrobromic acid, sulfuric acid, phosphoric acid, nitric acid, and the like), as well as salts formed with organic acids such as acetic acid, oxalic acid, tartaric acid, succinic acid, malic acid, fumaric acid, maleic acid, ascorbic acid, benzoic acid, tannic acid, palmoic acid, alginic acid, polyglutamic acid, naphthalene sulfonic acid, methane sulfonic acid, naphthalene disulfonic acid, and poly-galacturonic acid, as well as salts formed with basic amino acids such as Lysine or Arginine. Additional examples of such salts can be found in Berge, et al., J. Pharm. Sci., 66, 1 (1977).

“Pharmaceutically acceptable carrier” means compounds and compositions that are of sufficient purity and quality for use in the formulation of a composition of the invention and that, when appropriately administered to an animal or human, do not produce an adverse reaction.

The invention further includes the process for making the composition comprising mixing one or more of the present compounds and an optional pharmaceutically acceptable carrier; and includes those compositions resulting from such a process, which process includes conventional pharmaceutical techniques.

The compositions include compositions suitable for oral, rectal, topical, parenteral (including subcutaneous, intramuscular, and intravenous), ocular (ophthalmic), pulmonary (nasal or buccal inhalation), or nasal administration, although the most suitable route in any given case will depend on the nature and severity of the conditions being treated and on the nature of the active ingredient. They may be conveniently presented in unit dosage form and prepared by any of the methods well-known in the art of pharmacy.

In practical use, a compound of Formulae I, II, IIa-IIf, III, or IIIa-IIIi can be combined as the active ingredient in admixture with a pharmaceutical carrier according to conventional pharmaceutical compounding techniques. The carrier may take a wide variety of forms depending on the form of preparation desired for administration, e.g., oral or parenteral (including intravenous). In preparing the compositions for oral dosage form, any of the usual pharmaceutical media may be employed, such as, for example, water, glycols, oils, alcohols, flavoring agents, preservatives, coloring agents and the like in the case of oral liquid preparations, such as, suspensions, elixirs and solutions; or carriers such as starches, sugars, microcrystalline cellulose, diluents, granulating agents, lubricants, binders, disintegrating agents and the like in the case of oral solid preparations such as, powders, hard and soft capsules and tablets, with the solid oral preparations being preferred over the liquid preparations.

Because of their ease of administration, tablets and capsules represent the most advantageous oral dosage unit form in which case solid pharmaceutical carriers are obviously employed. If desired, tablets may be coated by standard aqueous or nonaqueous techniques. Such compositions and preparations should contain at least 0.1 percent of the active compound. The percentage of active compound, a pharmaceutically acceptable salt, or composition thereof, in these compositions may, of course, be varied and may conveniently be between about 2 percent to about 60 percent by weight. The amount of active compound in such therapeutically useful compositions is such that an effective dosage will be obtained. The active compounds can also be administered intranasally as, for example, liquid drops or spray.

The tablets, pills, capsules, and the like may also contain a binder such as gum tragacanth, acacia, corn starch or gelatin; excipients such as dicalcium phosphate; a disintegrating agent such as corn starch, potato starch, alginic acid; a lubricant such as magnesium stearate; and a sweetening agent such as sucrose, lactose or saccharin. When a dosage unit form is a capsule, it may contain, in addition to materials of the above type, a liquid carrier such as a fatty oil.

Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavoring such as cherry or orange flavor.

Compounds of the invention may also be administered parenterally. Solutions or suspensions of these active compounds can be prepared in water suitably mixed with a surfactant such as hydroxy-propylcellulose. Dispersions can also be prepared in glycerol, liquid polyethylene glycols and mixtures thereof in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), suitable mixtures thereof, and vegetable oils.

Any suitable route of administration may be employed for providing a mammal, especially a human, with an effective dose of a compound of the present invention. For example, oral, rectal, topical, parenteral, ocular, pulmonary, nasal, and the like may be employed. Dosage forms include tablets, troches, dispersions, suspensions, solutions, capsules, creams, ointments, aerosols, and the like. Preferably compounds of the present invention are administered orally.

The effective dosage of active ingredient employed may vary depending on the particular compound employed, the mode of administration, the condition being treated and the severity of the condition being treated. Such dosage may be ascertained readily by a person skilled in the art.

When treating hyperproliferative or inflammatory diseases for which compounds of the invention are indicated, generally satisfactory results are obtained when the compounds of the present invention are administered at a daily dosage of from about 0.1 milligram to about 100 milligram per kilogram of animal body weight, preferably given as a single daily dose or in divided doses two to six times a day, or in sustained release form. For most large mammals, the total daily dosage is from about 1.0 milligrams to about 1000 milligrams, preferably from about 1 milligram to about 50 milligrams. In the case of a 70 kg adult human, the total daily dose will generally be from about 7 milligrams to about 350 milligrams. This dosage regimen may be adjusted to provide the optimal therapeutic response.

The present invention comprises processes for the preparation of a compound of Formulae I, II, IIa-IIf, III, or IIIa-IIIi.

The compounds according to the invention can be prepared according to the procedures of the following Schemes and Examples, using appropriate materials and are further exemplified by the following specific examples. Moreover, by utilizing the procedures described herein, in conjunction with ordinary skills in the art, additional compounds of the present invention claimed herein can be readily prepared. The compounds illustrated in the examples are not, however, to be construed as forming the only genus that is considered as the invention. The examples further illustrate details for the preparation of the compounds according to Formulae I, II, IIa-IIf, III, or IIIa-IIIi. Those skilled in the art will readily understand that known variations of the conditions and processes of the following preparative procedures can be used to prepare these compounds. The instant compounds are generally isolated in the form of their pharmaceutically acceptable salts, such as those described above. The amine-free bases corresponding to the isolated salts can be generated by neutralization with a suitable base, such as aqueous sodium bicarbonate, sodium carbonate, sodium hydroxide and potassium hydroxide, and extraction of the liberated amine-free base into an organic solvent, followed by evaporation. The amine-free base, isolated in this manner, can be further converted into another pharmaceutically acceptable salt by dissolution in an organic solvent, followed by addition of the appropriate acid and subsequent evaporation, precipitation or crystallization.

### General Synthetic Procedures

An illustration of the preparation of compounds according to Formulae I, II, IIa-IIc, III, or IIIa-IIIi is shown in Schemes 1-6. Unless otherwise indicated in the schemes, the variables have the same meaning as described above. Specific conditions for the reactions shown in the following schemes are detailed in the Examples.

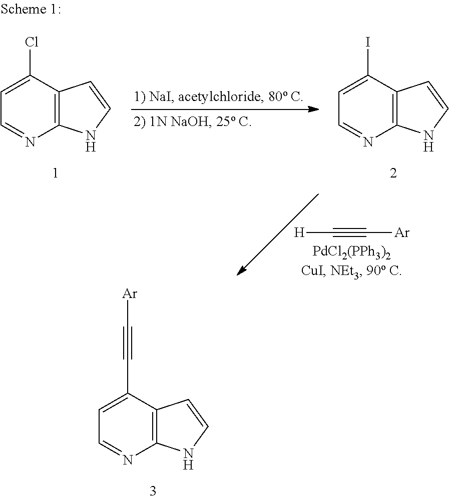

The 1H-pyrrolo[2,3-b]pyridine (3) can be prepared by the palladium mediated Sonagashira coupling outlined in Scheme 1.

Alternatively, 1H-pyrrolo[2,3-b]pyridine (3) can be prepared by the process outlined in Scheme 2.

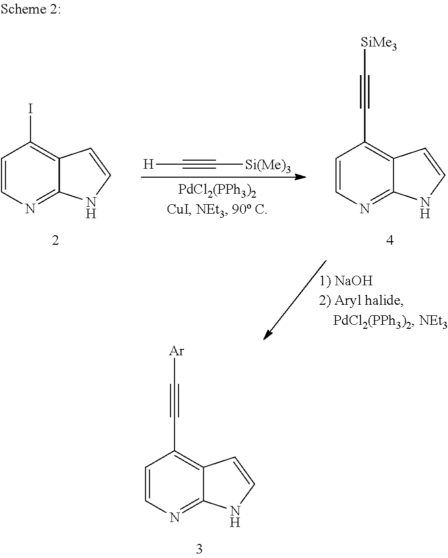

Similar to the process illustrated in Scheme 1, alternate routes for the Sonagashira couplings outlined in Scheme 2 may be employed by those skilled in the art.

The synthesis of the 7H-pyrrolo[2,3-d]pyrimidine (6) can be accessed in a process analogous to those used in Scheme 1 or Scheme 2. For the examples illustrated below, the synthetic route outlined in Scheme 3 was the most commonly used.

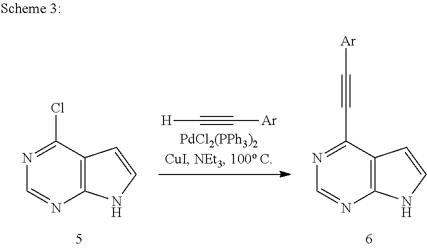

A route for the synthesis of 1H-pyrrolo[2,3-b]pyridine derivatives (8) is illustrated in Scheme 4.

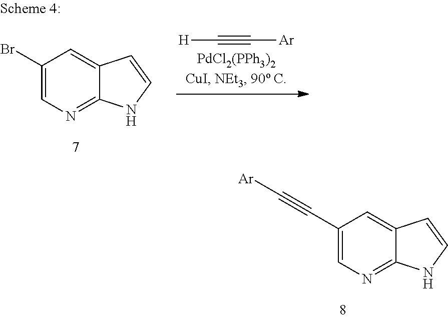

The synthesis of the tetrahydro-[1,8]naphthyridines (11) (Scheme 5) can be accessed via a synthetic route analogous to that illustrated in Scheme 1.

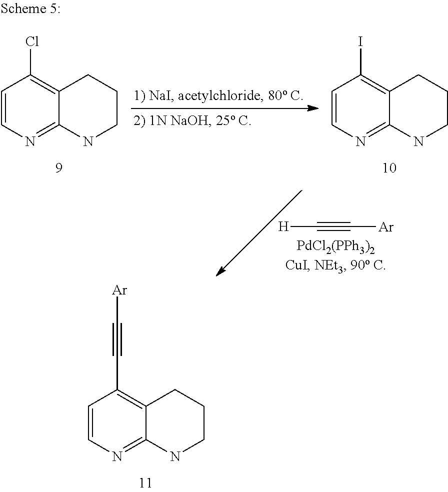

The tetrahydro[1]benzothieno[2,3-d]pyrimidines (13) are readily obtained via a palladium mediated coupling similar to that employed in Scheme 1.

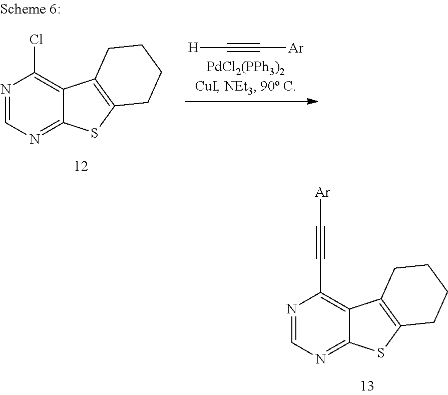

As can be appreciated by the skilled artisan, the above synthetic schemes are not intended to comprise a comprehensive list of all means by which the compounds described and claimed in this application may be synthesized. For example, in any of the above schemes, the substituents on Ar may be manipulated by standard techniques know by those of skill in the art. Additionally, further synthetic methods will be evident to those of ordinary skill in the art. The various synthetic steps described above may be performed in an alternate sequence or order to give the desired compounds. Furthermore, synthetic chemistry transformations and protecting group methodologies (protection and de-protection) useful in synthesizing the inhibitor compounds of the present invention are known in the art.

The compounds of the invention can be prepared according to the procedures detailed in the Schemes and Examples, using appropriate materials and are further exemplified by the following specific examples. Moreover, by utilizing the procedures described herein, in conjunction with ordinary skills in the art, additional compounds of the present invention claimed herein can be readily prepared. The compounds illustrated in the examples are not, however, to be construed as forming the only genus that is considered as the invention. The examples further illustrate details for the preparation of the compounds according to the invention. Those skilled in the art will readily understand that known variations of the conditions and processes of the following preparative procedures can be used to prepare these compounds. The instant compounds are generally isolated in the form of their pharmaceutically acceptable salts, such as those described above. The amine-free bases corresponding to the isolated salts can be generated by neutralization with a suitable base, such as aqueous sodium bicarbonate, sodium carbonate, sodium hydroxide and potassium hydroxide, and extraction of the liberated amine-free base into an organic solvent, followed by evaporation. The amine-free base, isolated in this manner, can be further converted into another pharmaceutically acceptable salt by dissolution in an organic solvent, followed by addition of the appropriate acid and subsequent evaporation, precipitation or crystallization.

The compounds of this invention may be modified by appending appropriate functionalities to enhance selective biological properties. Such modifications are known in the art and include those which increase biological penetration into a given biological compartment (e.g., blood, lymphatic system, central nervous system), increase oral bioavailability, increase solubility to allow administration by injection, alter metabolism and alter rate of excretion.

### Analytical Methodology

Analytical LC/MS was performed using the following two methods:

### Method A:

A Discovery® C¹⁸, 5 μm, 3×30 mm column was used at a flow rate of 400 μL/min, sample loop 5 μL, mobile phase: (A) methanol with 0.1% formic acid, mobile phase, (B) water with 0.1% formic acid; retention times are given in minutes. Method details: (I) runs on a Quaternary Pump G1311A (Agilent) with UV/Vis diode array detector G1315B (Agilent) and Finnigan LCQ Duo MS detector in ESI+modus with UV-detection at 254 and 280 nm with a gradient of 15-95% (B) in a 3.2 min linear gradient (II) hold for 1.4 min at 95% (B) (III) decrease from 95-15% (B) in a 0.1 min linear gradient (IV) hold for 2.3 min at 15% (B).

### Method B:

A Waters Symmetry® C¹⁸, 3.5 μm, 4.6×75 mm column at a flow rate of 400 μL/min, sample loop 5 μL, mobile phase (A) is methanol with 0.1% formic acid, mobile phase (B) is water with 0.1% formic acid; retention times are given in minutes. Methods details: (I) runs on a Binary Pump G1312A (Agilent) with UV/Vis diode array detector G1315B (Agilent) and Applied Biosystems API3000 MS detector in ESI+modus with UV-detection at 254 and 280 nm with a gradient of 20-95% (B) in a 10 min linear gradient (II) hold for 1 min at 95% (B) (III) decrease from 95-20% (B) in a 0.2 min linear gradient (IV) hold for 3.8 min at 20% (B).

If desired, isomers can be separated by methods well known in the art, e.g., by liquid chromatography. Additionally, enantiomers can be separated by methods well known in the art, i.e., by using chiral stationary phase liquid chromatography. Furthermore, enantiomers may be isolated by converting them into diastereomers, i.e., coupling with an enantiomerically pure auxiliary compound, subsequent separation of the resulting diastereomers and cleavage of the auxiliary residue. Alternatively, any enantiomer of a compound of the present invention may be obtained from stereoselective synthesis using optically pure starting materials.

### EXPERIMENTAL SECTION

### Abbreviations

ATP Adenosine-5′-triphosphate
CDCl₃ chloroform-d
CuI copper iodide
DMF dimethylformamide
DMSO dimethylsulfoxide
DMSO-d₆ d₆-dimethylsulfoxide
DTT 1,4-dithio-DL-threitol
EtOAc ethyl acetate
g gram
h hour
H₂O water
HCl hydrochloric acid
HEPES 4-(2-hydroxyethyl)-1-piperazineethanesulfonic acid
HPLC high perfomrance liquid chromatography
Hz hertz
K₂CO₃ potassium carbonate
LC/MS liquid chromatography/mass spectrometry
mg milligram
MgCl₂ magnesium chloride
mL milliliter
μl microliter
mmol millimole
MS mass spectrometry
N normal
NaHCO₃ sodium bicarbonate
NaI sodium iodide
NaOH sodium hydroxide
NEt₃ triethylamine
NMR nuclear magnetic resonance
° C. degrees celcius
PdCl₂(PPh₃)₂ palladium chloride bis(triphenylphosphine)
RT retention time
tert tertiary
THF tetrahydrofuran
TMS trimethylsilane

The following examples contain detailed descriptions of the methods of preparation of compounds of Formulae I, II, IIa-IIf, III, or IIIa-IIIi. These detailed descriptions fall within the scope, and serve to exemplify, the above described General Synthetic Procedures which form part of the invention. These detailed descriptions are presented for illustrative purposes only and are not intended as a restriction on the scope of the invention.

Unless otherwise noted, all materials were obtained from commercial suppliers and used without further purification. Unless otherwise noted, all non-aqueous reactions were carried out either under an argon or nitrogen atmosphere with commercial dry solvents. Compounds were purified using flash column chromatography using Merck silica gel 60 (230-400 mesh) or Biotage pre-packed column The ¹H-NMR spectra were recorded on a Joel ECP-400 (400 MHz for ¹H-NMR) using d₆-dimethylsulfoxide, d₄-methanol or CDCl₃ as solvent; chemical shifts are reported in ppm relative to tetramethylsilane.

### Example 1

### 4-(phenylethynyl)-1H-pyrrolo[2,3-b]pyridine

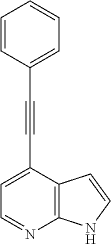

### Intermediate 1.1: 4-iodo-1H-pyrrolo[2,3-b]pyridine

Acetyl chloride (2.34 mL, 2.57 g, 32.8 mmol) was added dropwise to a solution of 4-chloro-1H-pyrrolo[2,3-b]pyridine (2.00 g, 13.1 mmol) and sodium iodide (13.8 g, 91.8 mmol) in acetonitrile (25 mL). The resulting suspension was heated at 80° C. for 7 days. After cooling to room temperature, the reaction mixture was concentrated under vacuo, and a saturated aqueous solution of potassium carbonate (50 mL) was added to the residue. The mixture was then extracted with dichloromethane (3×50 mL), the combined organic phase was washed with a saturated solution of sodium bisulfite (2×50 mL) and brine (50 mL), dried over sodium sulfate and concentrated under vacuo. The residue was dissolved in THF (25 mL) and added to an aqueous solution 1N of sodium hydroxide (15 mL). The resulting solution was stirred at 25° C. for 3 h. The reaction mixture was then concentrated under vacuo, and water (100 mL) was added to the residue. The mixture was extracted with dichloromethane (3×50 mL), the combined organic phase was washed with brine (50 mL), dried over sodium sulfate and concentrated under vacuo. The residue was purified by chromatography on a SP1 Biotage system, using hexanes and ethyl acetate as eluents to afford the title compound (1.26 g, 39%) as a yellow solid (HPLC: 66%, RT: 5.77 min) ¹H NMR (CDCl₃) δ=11.77 (br s, 1H), 7.94 (d, J=5.1 Hz, 1H), 7.51 (d, J=5.1 Hz, 1H), 7.44 (d, J=3.7 Hz, 1H), 6.41 (d, J=3.3 Hz, 1H); MS (m/z) 245 [M+H]⁺ (¹²⁷I).

### Example 1

### 4-(phenylethynyl)-1H-pyrrolo[2,3-b]pyridine

Dichloro bis(triphenylphosphine) palladium (II) (5.8 mg, 0.0082 mmol), copper (I) iodide (3.1 mg, 0.016 mmol) and triethylamine (288 μl, 207 mg, 2.05 mmol) were added to a solution of intermediate 1.1 (100 mg, 0.410 mmol) and phenylacetylene (89.1 μl, 83.7 mg, 0.820 mmol) in 1,4-dioxane (2 mL), and placed in a sealable tube. Nitrogen gas was bubbled in the reaction mixture for 5 min, before the tube was sealed and the reaction mixture was heated at 90° C. for 2 h. After cooling, the brown solution was filtered through Celite and concentrated under vacuo. The residue was purified by chromatography on a SP1 Biotage system, using hexanes and ethyl acetate as eluents to afford the title compound (79 mg, 88%) as a yellow solid (HPLC: 99%, RT: 6.69 min). ¹H NMR (DMSO-d₆) δ=11.94 (br s, 1H), 8.24 (d, J=4.8 Hz, 1H), 7.70-7.66 (m, 2H), 7.61 (dd, J=3.3, 2.6 Hz, 1H), 7.50-7.46 (m, 3H), 7.22 (d, J=4.8 Hz, 1H), 6.65 (dd, J=3.3, 1.8 Hz, 1H); MS (m/z) 219 [M+H]⁺.

### Example 2

### 4-[(3-chlorophenyl)ethynyl]-1H-pyrrolo[2,3-b]pyridine

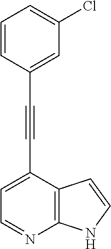

The title compound was obtained in 83% yield from intermediate 1.1 and 1-chloro-3-ethynylbenzene following the procedure described for example 1. (HPLC: 99%, RT: 7.24 min) ¹H NMR (DMSO-d₆) δ=11.95 (br s, 1H), 8.25 (d, J=4.8 Hz, 1H), 7.80 (dd, J=1.8, 1.5 Hz, 1H), 7.66 (td, J=7.3, 1.5 Hz, 1H), 7.63 (dd, J=3.3, 2.6 Hz, 1H), 7.58-7.48 (m, 2H), 7.23 (d, J=4.8 Hz, 1H), 6.70 (dd, J=3.7, 1.8 Hz, 1H); MS (m/z) 253 [M+H]⁺ (³⁵Cl).

### Example 3

### 4-(pyridin-2-ylethynyl)-1H-pyrrolo[2,3-b]pyridine

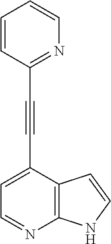

The title compound was obtained in 42% yield from intermediate 1.1 and 2-ethynylpyridine following the procedure described for example 1. (HPLC: 99%, RT: 4.98 min) ¹H NMR (CDCl₃) δ=8.69 (br s, 1H), 7.75 (td, J=7.7, 1.5 Hz, 1H), 7.65 (d, J=8.1 Hz, 1H), 7.47 (d, J=3.7 Hz, 1H), 7.37 (br s, 1H), 7.32 (ddd, J=7.5, 4.9, 1.1 Hz, 1H), 6.83 (br s, 1H); MS (m/z) 220 [M+H]⁺.

### Example 4

### 4-[(4-methoxyphenyl)ethynyl]-1H-pyrrolo[2,3-b]pyridine

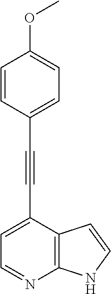

The title compound was obtained in 50% yield from intermediate 1.1 and 4-ethynylanisole following the procedure described for example 1. (HPLC: 99%, RT: 6.65 min) ¹H NMR (DMSO-d₆) δ=11.89 (br s, 1H), 8.22 (d, J=5.1 Hz, 1H), 7.62 (d, J=8.8 Hz, 2H), 7.58 (dd, J=3.3, 2.6 Hz, 1H), 7.17 (d, J=4.8 Hz, 1H), 7.03 (d, J=8.8 Hz, 2H), 6.63 (dd, J=3.3, 1.8 Hz, 1H), 3.82 (s, 3H); MS (m/z) 249 [M+H]⁺.

### Example 5

### 4-[(2,4-difluorophenyl)ethynyl]-1H-pyrrolo[2,3-b]pyridine

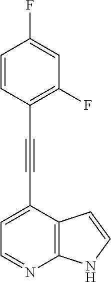

The title compound was obtained in 87% yield from intermediate 1.1 and 1-ethynyl-2,4-difluorobenzene following the procedure described for example 1. (HPLC: 91%, RT: 6.77 min) ¹H NMR (DMSO-d₆) δ=11.98 (br s, 1H), 8.26 (d, J=4.8 Hz, 1H), 7.84 (ddd, J=15.0, 8.4, 6.6 Hz, 1H), 7.63 (dd, J=3.3, 2.6 Hz, 1H), 7.50 (td, J=9.7, 2.6 Hz, 1H), 7.27-7.21 (m, 2H), 6.60 (dd, J=3.3, 1.8 Hz, 1H); MS (m/z) 255 [M+H]⁺.

### Example 6

### 4-(pyridin-4-ylethynyl)-1H-pyrrolo[2,3-b]pyridine

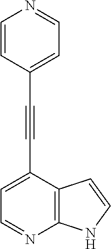

The title compound was obtained in 38% yield from intermediate 1.1 and 4-ethynylpyridine hydrochloride following the procedure described for example 1. (HPLC: 88%, RT: 4.85 min) ¹H NMR (DMSO-d₆) δ=12.01 (br s, 1H), 8.69 (dd, J=4.4, 1.5 Hz, 2H), 8.28 (d, J=4.8, 1H), 7.67-7.65 (m, 3H), 7.28 (d, J=5.1 Hz, 1H), 6.68 (dd, J=3.5, 1.8 Hz, 1H); MS (m/z) 220 [M+H]⁺.

### Example 7

### 3-(1H-pyrrolo[2,3-b]pyridin-4-ylethynyl)aniline

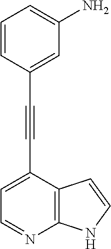

The title compound was obtained in 63% yield from intermediate 1.1 and 3-ethynylaniline following the procedure described for example 1. (HPLC: 99%, RT: 4.75 min) ¹H NMR (DMSO-d₆) δ=11.91 (br s, 1H), 8.22 (d, J=5.1, 1H), 7.59 (dd, J=3.3, 2.9 Hz, 1H), 7.17 (d, J=4.8 Hz, 1H), 7.10 (t, J=7.7 Hz, 1H), 6.83 (t, J=1.8 Hz, 1H), 6.78 (dt, J=7.3, 1.5 Hz, 1H), 6.65 (dd, J=8.1, 2.2 Hz, 1H), 6.57 (dd, J=3.3, 1.8 Hz, 1H), 5.32 (br s, 2H); MS (m/z) 234 [M+H]⁺.

### Example 8

### N-[3-(1H-pyrrolo[2,3-b]pyridin-4-ylethynyl)phenyl]acetamide

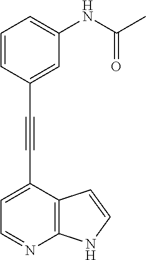

acetyl chloride (0.107 mL, 118 mg, 1.50 mmol) was carefully added to a solution of 3-(1H-pyrrolo[2,3-b]pyridin-4-ylethynyl)aniline (example 7, 70.0 mg, 0.300 mmol) in pyridine (2 mL). The yellow solution was stirred at 25° C. overnight, and then concentrated under vacuo. The residue was dissolve in THF (2 mL), a 1 N solution of sodium hydroxide (2 mML) was added, and the resulting mixture was stirred at 25° C. for 2 h, before being concentrated under vacuo. The residue was purified by chromatography on a SP1 Biotage system, using hexanes and ethyl acetate as eluents to afford the title compound (49 mg, 59%) as a white solid (HPLC: 99%, RT: 5.54 min) ¹H NMR (DMSO-d₆) δ=11.94 (br s, 1H), 10.12 (br s, 1H), 8.24 (d, J=4.8 Hz, 1H), 8.13 (dd, J=8.8, 5.5 Hz, 1H), 7.96 (dd, J=1.8, 1.5 Hz, 1H), 7.61 (dd, J=3.3, 2.9 Hz, 1H), 7.59 (ddd, J=8.1, 2.2, 1.5 Hz, 1H), 7.40 (dd, J=8.1, 7.7 Hz, 1H), 7.34 (dt, J=7.7, 1.5 Hz, 1H), 7.22 (d, J=4.8 Hz, 1H), 6.60 (dd, J=3.3, 1.8 Hz, 1H), 2.08 (s, 3H); MS (m/z) 276 [M+H]⁺.

### Example 9

### N,N-dimethyl-4-(1H-pyrrolo[2,3-b]pyridin-4-ylethynyl)aniline

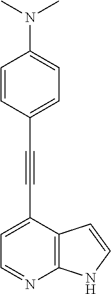

The title compound was obtained in 16% yield from intermediate 1.1 and 1-ethynyl-4-dimethylaniline following the procedure described for example 1. (HPLC: 99%, RT: 6.80 min) ¹H NMR (DMSO-d₆) δ=12.38 (br s, 1H), 8.30 (d, J=5.1 Hz, 1H), 7.68 (dd, J=3.3, 2.6 Hz, 1H), 7.54 (d, J=8.8 Hz, 2H), 7.30 (d, J=5.5 Hz, 1H), 6.80 (d, J=8.8 Hz, 2H), 6.77 (d, J=3.7, 2.2 Hz, 1H), 3.00 (s, 6H); MS (m/z) 262 [M+H]⁺.

### Example 10

### 4-fluoro-2-(1H-pyrrolo[2,3-b]pyridin-4-ylethynyl)benzonitrile

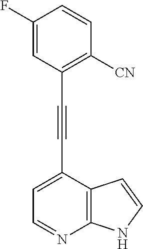

### Intermediate 10.1: 4-[(trimethylsilyl)ethynyl]-1H-pyrrolo[2,3-b]pyridine

Intermediate 10.1 was obtained in 84% yield from intermediate 1.1 and ethynyltrimethylsilane following the procedure described for example 1. (HPLC: 99%, RT: 6.80 min) ¹H NMR (DMSO-d₆) δ=11.92 (br s, 1H), 8.19 (d, J=4.8 Hz, 1H), 7.58 (dd, J=3.3, 2.6 Hz, 1H), 7.11 (d, J=4.8 Hz, 1H), 6.46 (dd, J=3.3, 1.8 Hz, 1H), 0.29 (s, 9H); MS (m/z) 215 [M+H]⁺.

### Intermediate 10.2: 4-ethynyl-1H-pyrrolo[2,3-b]pyridine

A solution 1 N of sodium hydroxide (14 mL, 14 mmol) was added to a solution of intermediate 10.1 (600 mg, 2.80 mmol) in methanol (20 mL). The resulting reaction mixture was stirred at 25° C. for 2 h, and then concentrated under vacuo. The residue was suspended in water (50 mL) and extracted with ethyl acetate (3×50 mL). The combined organic phase was washed with brine (50 mL), dried over sodium sulfate and concentrated under vacuo to afford the title compound (397 mg, 99%) as a yellow solid (HPLC: 66%, RT: 4.59 min) ¹H NMR (DMSO-d₆) δ=11.92 (br s, 1H), 8.20 (d, J=5.1 Hz, 1H), 7.58 (dd, J=3.3, 2.6 Hz, 1H), 7.15 (d, J=5.1 Hz, 1H), 6.50 (dd, J=3.3, 1.8 Hz, 1H), 4.67 (s, 1H); MS (m/z) 143 [M+H]⁺.

### Example 10

### 4-fluoro-2-(1H-pyrrolo[2,3-b]pyridin-4-ylethynyl)benzonitrile

Dichloro bis(triphenylphosphine) palladium (II) (29.6 mg, 0.0422 mmol), and triethylamine (742 μl, 534 mg, 5.28 mmol) were added to a solution of intermediate 10.2 (150 mg, 1.06 mmol) and 2-bromo-4-fluorobenzonitrile (1.06 g, 5.28 mmol) in 1,4-dioxane (4 mL), and placed in a sealable tube. Nitrogen gas was bubbled in the reaction mixture for 5 min, before the tube was sealed and the reaction mixture was heated at 80° C. for 2 h. After cooling, the brown solution was filtered through Celite and concentrated under vacuo. The residue was purified by chromatography on a SP1 Biotage system, using hexanes and ethyl acetate as eluents to afford the title compound (33 mg, 12%) as a yellow solid (HPLC: 99%, RT: 6.20 min) ¹H NMR (DMSO-d₆) δ=12.05 (br s, 1H), 8.31 (d, J=5.1 Hz, 1H), 8.13 (dd, J=8.8, 5.5 Hz, 1H), 7.90 (dd, J=9.3, 2.6 Hz, 1H), 7.69 (dd, J=3.3, 2.6 Hz, 1H), 7.59 (td, J=8.4, 2.6 Hz, 1H), 7.27 (d, J=4.8 Hz, 1H), 6.78 (dd, J=3.7, 1.8 Hz, 1H); MS (m/z) 262 [M+H]⁺.

### Example 11

### 2-[2-(1H-pyrrolo[2,3-b]pyridin-4-ylethynyl)phenyl]ethanol

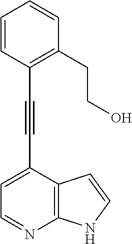

The title compound was obtained in 46% yield from intermediate 10.2 and 2-bromophenylethanol following the procedure described for example 10. (HPLC: 99%, RT: 2.99 min) ¹H NMR (DMSO-d₆) δ=11.94 (br s, 1H), 8.69 (dd, J=4.4, 1.5 Hz, 2H), 8.25 (d, J=5.1, 1H), 7.65-7.60 (m, 2H), 7.40 (d, J=4.0 Hz, 2H), 7.35-7.28 (m, 1H), 7.22 (d, J=4.8 Hz, 1H), 6.65 (dd, J=3.3, 1.8 Hz, 1H), 4.82 (t, J=5.1 Hz, 1H), 3.74 (ddd, J=7.3, 7.9, 5.1 Hz, 2H), 3.07 (t, J=7.3 Hz, 2H); MS (m/z) 263 [M+H]⁺.

### Example 12

Tert-butyl 2-(1H-pyrrolo[2,3-b]pyridin-4-ylethynyl)benzylcarbamate

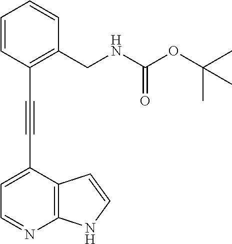

The title compound was obtained in 29% yield from intermediate 10.2 and tert-butyl 2-bromobenzylcarbamate following the procedure described for example 10. (HPLC: 99%, RT: 6.52 min) ¹H NMR (DMSO-d₆) δ=11.94 (br s, 1H), 8.25 (d, J=5.1 Hz, 1H), 7.65 (d, J=7.7 Hz, 1H), 7.62 (t, J=2.9 Hz, 1H), 7.52-7.45 (m, 2H), 7.39-7.32 (m, 2H), 7.26 (d, J=5.1 Hz, 1H), 6.64 (dd, J=3.3, 1.8 Hz, 1H), 4.46 (d, J=5.9 Hz, 2H), 1.41 (s, 9H); MS (m/z) 348 [M+H]⁺.

### Example 13

### 2-(1H-pyrrolo[2,3-b]pyridin-4-ylethynyl)benzylamine

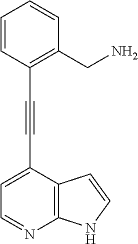

To a solution of tert-butyl 2-(1H-pyrrolo[2,3-b]pyridin-4-ylethynyl)benzylcarbamate (example 12, 119 mg, 0.343 mmol) in methanol (3 mL) and ether (6 mL) was added a 2 N solution of hydrogen chloride (1.71 mL, 3.43 mmol) in ether. The colorless solution was stirred at 25° C. overnight, and a precipitate formed slowly. The yellow solid was filtered, washed with ether and dried in vacuo to afford to afford the title compound (82 mg, 84%) as a hydrochloride salt (HPLC: 99%, RT: 0.45 min). ¹H NMR (DMSO-d₆) δ=12.15 (br s, 1H), 8.60 (br s, 2H), 8.31 (d, J=5.1 Hz, 1H), 7.78 (dd, J=7.7, 1.5 Hz, 1H), 7.70 (d, J=7.7 Hz, 1H), 7.68 (t, J=2.9 Hz, 1H), 7.58 (td, J=7.7, 1.5 Hz, 1H), 7.51 (td, J=7.7, 1.1 Hz, 1H), 7.34 (dd, J=5.1, 0.7 Hz, 1H), 6.74 (m, 1H), 4.38-4.33 (m, 2H); MS (m/z) 248 [M+H]⁺.

### Example 14

### (1S)-1-[2-(1H-pyrrolo[2,3-b]pyridin-4-ylethynyl)phenyl]ethanol

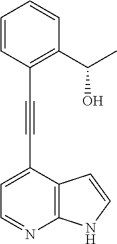

The title compound was obtained in 38% yield from intermediate 10.2 and (1S)-1-(2-bromophenyl)ethanol following the procedure described for example 10. (HPLC: RT: 5.91 min) ¹H NMR (DMSO-d₆) δ=11.96 (br s, 1H), 8.25 (d, J=4.8, 1H), 7.66 (d, J=8.4 Hz, 1H), 7.63 (dd, J=3.7, 2.6 Hz, 1H), 7.60 (dd, J=7.7, 1.1 Hz, 1H), 7.48 (td, J=7.7, 1.5 Hz, 1H), 7.33 (td, J=7.5, 1.5 Hz, 1H), 7.21 (d, J=5.1 Hz, 1H), 6.59 (dd, J=3.3, 2.0 Hz, 1H), 5.41 (d, J=4.0 Hz, 1H), 5.34-5.28 (m, 1H), 1.44 (d, J=6.6 Hz, 3H); MS (m/z) 363 [M+H]⁺.

### Example 15

### 4-[(2,6-dichlorophenyl)ethynyl]-1H-pyrrolo[2,3-b]pyridine

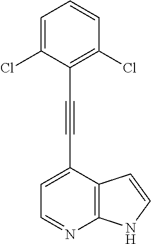

The title compound was obtained in 61% yield from intermediate 10.2 and 1,3-dichloro-2-iodobenzene following the procedure described for example 10.

(HPLC: 99%, RT: 7.25 min) ¹H NMR (DMSO-d₆) δ=12.03 (br s, 1H), 8.29 (d, J=4.8, 1H), 7.67 (d, J=8.4 Hz, 2H), 7.66 (dd, J=3.7, 2.6 Hz, 1H), 7.51 (dd, J=8.6, 7.7 Hz, 1H), 7.26 (d, J=5.1 Hz, 1H), 6.63 (dd, J=3.3, 1.8 Hz, 1H); MS (m/z) 287 [M+H]⁺ (³⁵Cl+³⁷Cl).

### Example 16

### 4-[(2-thien-2-ylphenyl)ethynyl]-1H-pyrrolo[2,3-b]pyridine

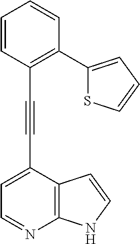

The title compound was obtained in 47% yield from intermediate 10.2 and 2-(2-bromophenyl)thiophene following the procedure described for example 10. (HPLC: 98%, RT: 7.20 min) ¹H NMR (DMSO-d₆) δ=11.95 (br s, 1H), 8.25 (d, J=5.1 Hz, 1H), 7.80 (dd, J=7.7, 1.5 Hz, 1H), 7.74-7.70 (m, 3H), 7.60 (dd, J=3.3, 2.6 Hz, 1H), 7.53 (td, J=7.7, 1.5 Hz, 1H), 7.44 (td, J=7.5, 1.5 Hz, 1H), 7.23 (dd, J=5.1, 3.7 Hz, 1H), 7.20 (d, J=4.8 Hz, 1H), 6.50 (dd, J=3.7, 1.8 Hz, 1H); MS (m/z) 301 [M+H]⁺.

### Example 17

### 4-{[2-(1,3-oxazol-5-yl)phenyl]ethynyl}-1H-pyrrolo[2,3-b]pyridine

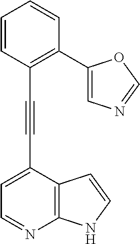

The title compound was obtained in 49% yield from intermediate 10.2 and 5-(2-bromophenyl)-1,3-oxazole following the procedure described for example 10. (HPLC: 99%, RT: 6.48 min) ¹H NMR (DMSO-d₆) δ=12.00 (br s, 1H), 8.61 (s, 1H), 8.29 (d, J=5.1 Hz, 1H), 7.99 (s, 1H), 7.85 (ddd, J=7.7, 2.2, 1.5 Hz, 2H), 7.65 (dd, J=3.3, 2.6 Hz, 1H), 7.60 (td, J=8.1, 1.5 Hz, 1H), 7.28 (d, J=4.8 Hz, 1H), 6.60 (dd, J=3.7, 1.8 Hz, 1H); MS (m/z) 286 [M+H]⁺.

### Example 18

### 4-(phenylethynyl)-7H-pyrrolo[2,3-d]pyrimidine

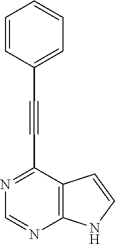

Dichloro bis(triphenylphosphine) palladium (II) (23 mg, 0.033 mmol), copper (I) iodide (12 mg, 0.065 mmol) and triethylamine (451 μl, 330 mg, 3.26 mmol) was added to a solution of 4-chloro-7H-pyrrolo[2,3-c]pyrimidine (100 mg, 0.651 mmol) and phenylacetylene (215 μl, 200 mg, 1.95 mmol) in DMF (2 mL), and placed in a sealable tube. Nitrogen gas was bubbled in the reaction mixture for 5 min, before the tube was sealed and the reaction mixture was heated at 100° C. for 2 h. After cooling to room temperature, the brown solution was filtered through Celite and concentrated under vacuo. The residue was purified by chromatography on a SP1 Biotage system, using hexanes and ethyl acetate as eluents to afford the title compound (82 mg, 57%) as a greenish solid (HPLC: 99%, RT: 7.09 min). ¹H NMR (DMSO-d₆) δ=12.38 (br s, 1H), 8.77 (s, 1H), 7.77-7.73 (m, 2H), 7.71 (dd, J=3.3, 2.6 Hz, 1H), 7.55-7.50 (m, 3H), 6.76 (dd, J=3.3, 1.8 Hz, 1H); MS (m/z) 220 [M+H]⁺.

### Example 19

### 4-(7H-pyrrolo[2,3-d]pyrimidin-4-ylethynyl)aniline

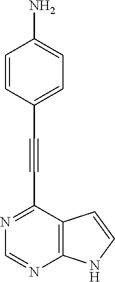

The title compound was obtained in 32% yield from 4-chloro-7H-pyrrolo[2,3-c]pyrimidine and 4-ethynylaniline following the procedure described for example 18. (HPLC: 99%, RT: 3.96 min) ¹H NMR (DMSO-d₆) δ=12.23 (br s, 1H), 8.68 (s, 1H), 7.62 (dd, J=3.3, 2.6 Hz, 1H), 7.38 (d, J=8.4 Hz, 2H), 6.67 (d, J=3.7, 1.8 Hz, 1H), 6.61 (d, J=8.4 Hz, 2H), 5.85 (br s, 2H); MS (m/z) 235 [M+H]⁺.

### Example 20

### N,N-dimethyl-4-(7H-pyrrolo[2,3-d]pyrimidin-4-ylethynyl)aniline

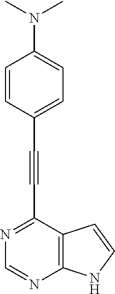

The title compound was obtained in 39% yield from 4-chloro-7H-pyrrolo[2,3-c]pyrimidine and 1-ethynyl-4-dimethylaniline following the procedure described for example 18. (HPLC: 99%, RT: 5.81 min) ¹H NMR (DMSO-d₆) δ=12.25 (br s, 1H), 8.69 (s, 1H), 7.63 (dd, J=3.3, 2.6 Hz, 1H), 7.53 (d, J=8.8 Hz, 2H), 6.77 (d, J=9.2 Hz, 2H), 6.71 (d, J=3.3, 1.8 Hz, 1H), 2.30 (s, 6H); MS (m/z) 263 [M+H]⁺.

### Example 21

### 5-(phenylethynyl)-1H-pyrrolo[2,3-b]pyridine

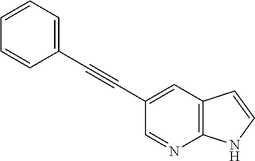

Dichloro bis(triphenylphosphine) palladium (II) (18 mg, 0.026 mmol), copper (I) iodide (10 mg, 0.053 mmol) and triethylamine (352 μl, 257 mg, 2.54 mmol) were added to a solution of 5-bromo-1H-pyrrolo[2,3-b]pyridine (100 mg, 0.507 mmol) and phenylacetylene (167 μl, 155 mg, 1.52 mmol) in 1,4-dioxane (2 mL) and placed in a sealable tube. Nitrogen was bubbled in the reaction mixture for 5 min, before the tube was sealed and the reaction mixture was heated at 90° C. overnight. After cooling to room temperature, the brown solution was filtered through Celite and concentrated under vacuo. The residue was purified by chromatography on a SP1 Biotage system, using hexanes and ethyl acetate as eluents to afford the title compound (23 mg, 21%) as a light yellow solid (HPLC: 99%, RT: 6.78 min) ¹H NMR (DMSO-d₆) δ=11.93 (br s, 1H), 8.40 (d, J=1.8 Hz, 1H), 8.18 (d, J=1.5 Hz, 1H), 7.59-7.55 (m, 3H), 7.47-7.39 (m, 3H), 6.50 (dd, J=3.3, 1.8 Hz, 1H); MS (m/z) 219 [M+H]⁺.

### Example 22

### 4-(1H-pyrrolo[2,3-b]pyridin-5-ylethynyl)aniline

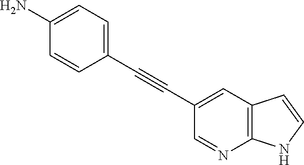

Piperidine (251 ml, 216 mg, 2.54 mmol), bis(benzonitrile)dichloropalladium(II) (3.9 mg, 0.010 mmol), copper (I) iodide (3.9 mg, 0.021 mmol) and di(tert-butyl)(2′,4′,6′-triisopropyl-1,1′-biphenyl-2-yl)phosphine (13 mg; 0.031 mmol) were added to a solution of 5-bromo-1H-pyrrolo[2,3-b]pyridine (100 mg, 0.507 mmol) and 4-ethynylaniline (89 mg, 0.76 mmol) in 1,4-dioxane (2 mL) and placed in a sealable tube. Nitrogen gas was bubbled in the reaction mixture for 5 min, before the tube was sealed and the reaction mixture was heated at 100° C. overnight. After cooling to room temperature, the brown solution was filtered through Celite and concentrated under vacuo. The residue was purified by chromatography on a SP1 Biotage system, using hexanes and ethyl acetate as eluents to afford the title compound (29 mg, 25%) as a yellow solid (HPLC: 99%, RT: 5.03 min) ¹H NMR (DMSO-d₆) δ=11.83 (br s, 1H), 8.30 (d, J=1.8 Hz, 1H), 8.05 (d, J=1.8 Hz, 1H), 7.52 (dd, J=3.3, 2.6 Hz, 1H), 7.21 (d, J=8.4 Hz, 2H), 6.56 (d, J=8.4 Hz, 2H), 6.46 (dd, J=3.3, 1.8 Hz, 1H), 5.54 (br s, 2H); MS (m/z) 234 [M+H]⁺.

### Example 23

### 2-(1H-pyrrolo[2,3-b]pyridin-5-ylethynyl)aniline

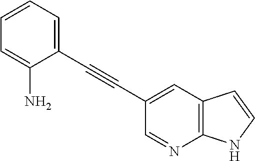

The title compound was obtained in 9% yield from 5-bromo-1H-pyrrolo[2,3-b]pyridine and 2-ethynylaniline following the procedure described for example 21. (HPLC: 97%, RT: 5.92 min) ¹H NMR (DMSO-d₆) δ=11.87 (br s, 1H), 8.44 (d, J=2.2 Hz, 1H), 8.21 (d, J=1.8 Hz, 1H), 7.54 (dd, J=3.1, 2.8 Hz, 1H), 7.24 (dd, J=7.5, 1.8 Hz, 1H), 7.07 (ddd, J=8.3, 7.2, 1.8 Hz, 1H), 6.73 (d, J=7.3 Hz, 1H), 6.54 (td, J=7.3, 1.1 Hz, 1H), 6.48 (dd, J=3.3, 1.8 Hz, 1H), 5.51 (br s, 2H); MS (m/z) 234 [M+H]⁺.

### Example 24

### 3-(1H-pyrrolo[2,3-b]pyridin-5-ylethynyl)aniline

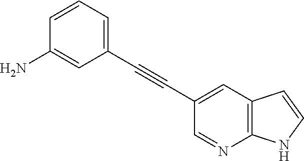

The title compound was obtained in 28% yield from 5-bromo-1H-pyrrolo[2,3-b]pyridine and 3-ethynylaniline following the procedure described for example 21. (HPLC: 99%, RT: 5.02 min) ¹H NMR (DMSO-d₆) δ=11.90 (br s, 1H), 8.35 (d, J=1.8 Hz, 1H), 8.13 (d, J=1.5 Hz, 1H), 7.55 (dd, J=3.3, 2.9 Hz, 1H), 7.24 (dd, J=7.5, 1.8 Hz, 1H), 7.05 (t, J=7.7 Hz, 1H), 6.74 (t, J=1.8 Hz, 1H), 6.69 (dt, J=7.3, 1.1 Hz, 1H), 6.59 (ddd, J=8.1, 2.6, 1.1 Hz, 1H), 6.48 (dd, J=3.7, 1.8 Hz, 1H), 5.26 (br s, 2H); MS (m/z) 234 [M+H]⁺.

### Example 25

### 5-{[2-(trifluoromethyl)phenyl]ethynyl}-1H-pyrrolo[2,3-b]pyridine

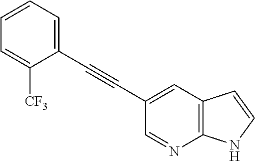

The title compound was obtained in 18% yield from 5-bromo-1H-pyrrolo[2,3-b]pyridine and 1-ethynyl-2-(trifluoromethyl)benzene following the procedure described for example 21. (HPLC: 99%, RT: 6.86 min) ¹H NMR (DMSO-d₆) δ=12.01 (br s, 1H), 8.38 (d, J=2.2 Hz, 1H), 8.17 (d, J=1.8 Hz, 1H), 7.84 (t, J=7.3 Hz, 2H), 7.74 (t, J=7.7 Hz, 1H), 7.62 (t, J=7.7 Hz, 1H), 7.59 (dd, J=3.3, 2.6 Hz, 1H), 6.69 (dt, J=7.3, 1.1 Hz, 1H), 6.54 (dd, J=3.3, 1.8 Hz, 1H); MS (m/z) 287 [M+H]⁺.

### Example 26

### 5-[(4-methoxyphenyl)ethynyl]-1H-pyrrolo[2,3-b]pyridine

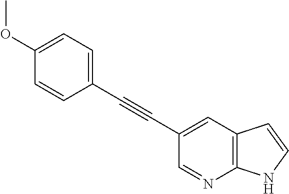

The title compound was obtained in 25% yield from 5-bromo-1H-pyrrolo[2,3-b]pyridine and 1-ethynyl-4-methoxybenzene following the procedure described for example 21. (HPLC: 99%, RT: 6.73 min) ¹H NMR (DMSO-d₆) δ=11.89 (br s, 1H), 8.36 (d, J=1.8 Hz, 1H), 8.13 (d, J=1.8 Hz, 1H), 7.55 (dd, J=3.3, 2.6 Hz, 1H), 7.51 (d, J=8.8 Hz, 2H), 6.99 (d, J=8.8 Hz, 2H), 6.48 (dd, J=3.3, 1.8 Hz, 1H), 3.80 (s, 3H); MS (m/z) 249 [M+H]⁺.

### Example 27

### 3,3-Dimethyl-N-[2-(1H-pyrrolo[2,3-b]pyridin-4-ylethynyl)-benzyl]-butyramide

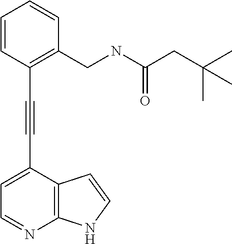

1-(3-Dimethylaminopropyl)-3-ethylcarbodiimide hydrochloride (41 mg, 0.21 mmol), 1-hydroxybenzotriazole (29 mg, 0.21 mmol) and N,N-diisopropylethylamine (146 μl, 114 mg, 0.88 mmol) were added to a solution of 3,3-dimethyl-butyric acid (22 μl, 21 mg, 0.18 mmol) in anhydrous DMF (2 mL). The reaction mixture was stirred at 25° C. for 15 minutes before 1-[2-(1H-pyrrolo[2,3-b]pyridin-4-ylethynyl)phenyl]methanamine (example 13, 50 mg, 0.18 mmol) was added. The resulting solution was stirred at 25° C. overnight and concentrated under vacuo. The residue was purified by chromatography on a SP1 Biotage system, using hexanes and ethyl acetate as eluents to afford the title compound (29 mg, 47%) as a white solid (HPLC: 98%, RT: 6.25 min) ¹H NMR (DMSO-d₆) δ=11.94 (br s, 1H), 8.33 (t, J=5.5 Hz, 1H), 8.25 (d, J=4.8 Hz, 1H), 7.67 (dd, J=7.5, 1.5 Hz, 1H), 7.62 (dd, J=3.3, 2.6 Hz, 1H), 7.46 (td, J=7.3, 1.5 Hz, 1H), 7.40 (d, J=6.6 Hz, 1H), 7.36 (td, J=7.3, 1.5 Hz, 1H), 7.25 (d, J=5.1 Hz, 1H), 6.64 (dd, J=3.3, 1.8 Hz, 1H), 4.59 (d, J=5.5 Hz, 2H), 2.08 (s, 2H), 0.98 (s, 9H); MS (m/z) 346 [M+H]⁺.

### Example 28

### N-[2-(1H-Pyrrolo[2,3-b]pyridin-4-ylethynyl)-benzyl]-benzamide

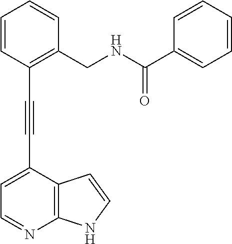

The title compound was obtained in 59% yield from 1-[2-(1H-pyrrolo[2,3-b]pyridin-4-ylethynyl)phenyl]methanamine (example 13) and benzoic acid following the procedure described for example 27. (HPLC: 99%, RT: 5.91 min). ¹H NMR (DMSO-d₆) δ=11.93 (br s, 1H), 9.13 (t, J=5.9 Hz, 1H), 8.25 (d, J=5.1 Hz, 1H), 7.96-7.93 (m, 2H), 7.69 (dd, J=7.7, 1.1 Hz, 1H), 7.61 (dd, J=3.3, 2.2 Hz, 1H), 7.58-7.35 (m, 6H), 7.26 (d, J=5.1 Hz, 1H), 6.66 (dd, J=3.1, 1.8 Hz, 1H), 4.82 (d, J=5.9 Hz, 2H); MS (m/z) 352 [M+H]⁺.

### Example 29

### 4-Dimethylamino-N-[2-(1H-pyrrolo[2,3-b]pyridin-4-ylethynyl)-benzyl]-benzamide

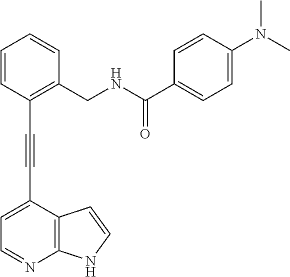

The title compound was obtained in 53% yield from 1-[2-(1H-pyrrolo[2,3-b]pyridin-4-ylethynyl)phenyl]methanamine (example 13) and 4-dimethylaminobenzoic acid following the procedure described for example 27. (HPLC: 99%, RT: 6.06 min) ¹H NMR (DMSO-d₆) δ=11.94 (br s, 1H), 8.77 (t, J=5.9 Hz, 1H), 8.25 (d, J=4.8 Hz, 1H), 7.82 (d, J=9.2 Hz, 2H), 7.67 (d, J=7.7 Hz, 1H), 7.62 (dd, J=3.3, 2.6 Hz, 1H), 7.44 (td, J=7.7, 1.1 Hz, 1H), 7.39-7.33 (m, 2H), 7.26 (d, J=5.1 Hz, 1H), 6.73 (d, J=9.2 Hz, 2H), 6.66 (dd, J=3.3, 1.8 Hz, 1H), 4.78 (d, J=5.9 Hz, 2H), 2.98 (s, 6H); MS (m/z) 395 [M+H]⁺.

### Example 30

### 4-Methoxy-N-[2-(1H-pyrrolo[2,3-b]pyridin-4-ylethynyl)-benzyl]-benzamide

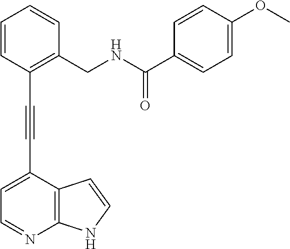

The title compound was obtained in 62% yield from 1-[2-(1H-pyrrolo[2,3-b]pyridin-4-ylethynyl)phenyl]methanamine (example 13) and 4-methoxybenzoic acid following the procedure described for example 27. (HPLC: 99%, RT: 5.96 min) ¹H NMR (DMSO-d₆) δ=11.94 (br s, 1H), 8.99 (t, J=5.9 Hz, 1H), 8.29 (d, J=5.1 Hz, 1H), 7.93 (d, J=8.8 Hz, 2H), 7.69 (d, J=7.7 Hz, 1H), 7.61 (dd, J=3.3, 2.6 Hz, 1H), 7.45 (td, J=7.7, 1.5 Hz, 1H), 7.41-7.34 (m, 2H), 7.26 (d, J=5.1 Hz, 1H), 7.02 (d, J=8.8 Hz, 2H), 6.66 (dd, J=3.3, 1.8 Hz, 1H), 4.80 (d, J=5.5 Hz, 2H), 3.82 (s, 3H); MS (m/z) 382 [M+H]⁺.

### Example 31

### 2,2,2-Trifluoro-N-[2-(1H-pyrrolo[2,3-b]pyridin-4-ylethynyl)-benzyl]-acetamide

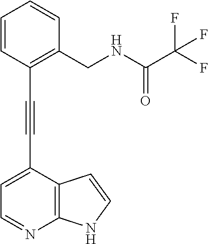

The title compound was obtained in 65% yield from intermediate 10.2 and 2,2,2-trifluoro-N-(2-iodobenzyl)acetamide following the procedure described for example 10. (HPLC: 99%, RT: 5.90 min) ¹H NMR (DMSO-d₆) δ=11.94 (br s, 1H), 10.09 (t, J=5.9, 1H), 8.26 (d, J=4.8 Hz, 1H), 7.71 (d, J=7.7 Hz, 1H), 7.62 (t, J=2.9 Hz, 1H), 7.50 (td, J=7.7, 1.5 Hz, 1H), 7.42 (t, J=7.7 Hz, 1H), 7.39 (d, J=7.7 Hz, 1H), 7.25 (d, J=5.1 Hz, 1H), 6.65 (dd, J=3.3, 1.8 Hz, 1H), 4.72 (d, J=5.9 Hz, 2H); MS (m/z) 344 [M+H]⁺.

### Example 32

### 1-Phenyl-3-[2-(1H-pyrrolo[2,3-b]pyridin-4-ylethynyl)-benzyl]-urea

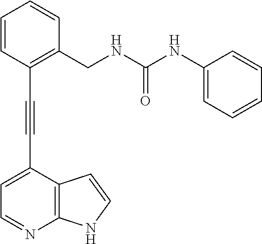

1-[2-(1H-Pyrrolo[2,3-b]pyridin-4-ylethynyl)phenyl]methanamine (example 13, 50 mg, 0.18 mmol), phenyl isocyanate (58 μl, 63 mg, 0.53 mmol) and N,N-diisopropylethylamine (146 μl, 114 mg, 0.88 mmol) were dissolved in anhydrous THF (1 mL) and stirred at 25° C. overnight. The reaction mixture was diluted with DMSO (5 mL) and a 5 N solution of sodium hydroxide (2 mL) was added to the solution and stirred at 25° C. for 1 h. The reaction mixture was further diluted with water (100 mL), and thereby forming a precipitate which was then filtered. The tan solid was triturated in dichloromethane and methanol and filtered to afford the title compound (12 mg, 19%) as a beige solid (HPLC: 98%, RT: 5.03 min) ¹H NMR (DMSO-d₆) δ=11.66 (br s, 1H), 8.75 (s, 1H), 8.20 (d, J=4.8 Hz, 1H), 7.85 (s, 1H), 7.60 (d, J=8.4 Hz, 2H), 7.46 (d, J=7.3 Hz, 1H), 7.41 (dd, J=3.3, 2.6 Hz, 1H), 7.33 (t, J=7.3 Hz, 3H), 7.08-7.00 (m, 3H), 6.93 (d, J=8.1 Hz, 1H), 6.27 (dd, J=3.3, 1.8 Hz, 1H), 5.17 (s, 2H); MS (m/z) 367 [M+H]⁺.

### Example 33

### 1-tert-Butyl-3-[2-(1H-pyrrolo[2,3-b]pyridin-4-ylethynyl)-benzyl]-urea

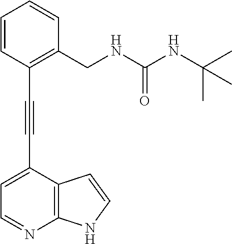

The title compound was obtained in 27% yield from 1-[2-(1H-pyrrolo[2,3-b]pyridin-4-ylethynyl)phenyl]methanamine (example 1) and tert-butyl isocyanate following the procedure described for example 32. (HPLC: 98%, RT: 6.03 min). ¹H NMR (DMSO-d₆) δ=11.92 (br s, 1H), 8.25 (d, J=4.8 Hz, 1H), 7.65 (d, J=7.7 Hz, 1H), 7.61 (t, J=2.9 Hz, 1H), 7.46 (t, J=7.3 Hz, 1H), 7.39 (d, J=7.3 Hz, 1H), 7.34 (t, J=7.3 Hz, 1H), 7.25 (d, J=4.8 Hz, 1H), 6.65 (dd, J=3.3, 1.8 Hz, 1H), 6.17 (t, J=5.9 Hz, 1H), 5.87 (s, 1H), 4.48 (d, J=5.9 Hz, 2H), 1.24 (s, 9H); MS (m/z) 347 [M+H]⁺.

### Example 34

### [2-(1H-Pyrrolo[2,3-b]pyridin-4-ylethynyl)-phenyl]-acetic acid

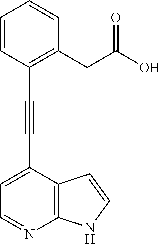

Dichloro bis(triphenylphosphine) palladium (II) (17 mg, 0.024 mmol), and triethylamine (335 μL, 241 mg, 2.39 mmol) were added to a solution of intermediate 10.2 (102 mg, 1.06 mmol) and (2-iodophenyl)acetic acid (125 mg, 0.48 mmol) in 1,4-dioxane (2 mL), and placed in a sealable tube. Nitrogen gas was bubbled in the reaction mixture for 5 min, before the tube was sealed and the reaction mixture was heated at 60° C. for 2 h. After cooling to room temperature, the brown solution was filtered through Celite and concentrated under vacuo. The residue was then dissolved in a solution 5 N of sodium hydroxide, washed with ethyl acetate (3×20 mL), neutralized with a solution 5 N of hydrochloric acid, and filtered to afford the title compound (60 mg, 45%) as a beige solid (HPLC: 99%, RT: 6.19 min) ¹H NMR (DMSO-d₆) δ=8.25 (d, J=4.8 Hz, 1H), 7.66 (d, J=7.3 Hz, 1H), 7.62 (dd, J=3.3, 2.6 Hz, 1H), 7.45-735 (m, 3H), 7.21 (d, J=5.1 Hz, 1H), 6.66 (dd, J=3.5, 1.8 Hz, 1H), 3.90 (s, 2H); MS (m/z) 277 [M+H]⁺.

### Example 35

### N-tert-Butyl-2-[2-(1H-pyrrolo[2,3-b]pyridin-4-ylethynyl)-phenyl]-acetamide

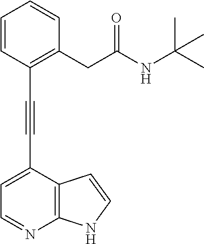

1-(3-Dimethylaminopropyl)-3-ethylcarbodiimide hydrochloride (30 mg, 0.16 mmol), 1-hydroxybenzotriazole (21 mg, 0.16 mmol) and N,N-diisopropylethylamine (108 μl, 84 mg, 0.65 mmol) were added to a solution of [2-(1H-pyrrolo[2,3-b]pyridin-4-ylethynyl)-phenyl]-acetic acid (example 34, 36 mg, 0.13 mmol) in anhydrous DMF (2 mL). The reaction mixture was stirred at 25° C. for 15 minutes before tert-butylamine (14 μl, 10 mg, 0.13 mmol) was added, and the resulting solution was then stirred at 25° C. overnight before concentrating under vacuo. The residue was purified by chromatography on a SP1 Biotage system, using hexanes and ethyl acetate as eluents to afford the title compound (6 mg, 14%) as a white solid (HPLC: 98%, RT: 5.89 min). ¹H NMR (DMSO-d₆) δ=11.91 (br s, 1H), 8.25 (d, J=4.8 Hz, 1H), 7.74 (s, 1H), 7.64 (d, J=7.7 Hz, 1H), 7.61 (dd, J=3.3, 2.9 Hz, 1H), 7.43-7.31 (m, 3H), 7.27 (d, J=4.8 Hz, 1H), 6.68 (dd, J=3.7, 1.8 Hz, 1H), 3.73 (s, 2H), 1.21 (s, 9H); MS (m/z) 332 [M+H]⁺.

### Example 36

### N-(4-Methoxybenzyl)-2-[2-(1H-pyrrolo[2,3-b]pyridin-4-ylethynyl)-phenyl]-acetamide

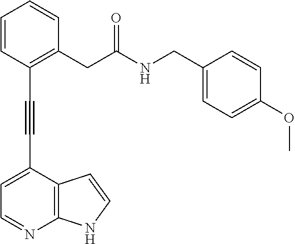

The title compound was obtained in 54% yield from [2-(1H-pyrrolo[2,3-b]pyridin-4-ylethynyl)-phenyl]-acetic acid (example 34) and 4-methoxybenzylamine following the procedure described for example 35. (HPLC: 99%, RT: 5.80 min). ¹H NMR (DMSO-d₆) δ=11.91 (br s, 1H), 8.46 (t, J=5.9 Hz, 1H), 8.22 (d, J=4.8 Hz, 1H), 7.66 (d, J=7.3 Hz, 1H), 7.59 (t, J=2.9 Hz, 1H), 7.44-7.33 (m, 3H), 7.19 (d, J=4.8 Hz, 1H), 7.13 (d, J=8.4 Hz, 2H), 6.69 (d, J=8.4 Hz, 2H), 6.65 (dd, J=3.3, 1.8 Hz, 1H), 4.21 (d, J=5.9 Hz, 2H), 3.84 (s, 2H), 3.66 (s, 3H); MS (m/z) 396 [M+H]⁺.

### Example 37

### N-(4-Methoxyphenyl)-2-[2-(1H-pyrrolo[2,3-b]pyridin-4-ylethynyl)-phenyl]-acetamide

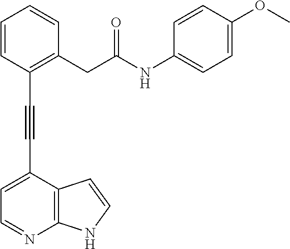

The title compound was obtained in 56% yield from [2-(1H-pyrrolo[2,3-b]pyridin-4-ylethynyl)-phenyl]-acetic acid (example 34) and p-anisidine following the procedure described for example 35. (HPLC: 99%, RT: 5.81 min). ¹H NMR (DMSO-d₆) δ=11.87 (br s, 1H), 10.14 (s, 1H), 8.18 (d, J=5.1 Hz, 1H), 7.68 (d, J=7.3 Hz, 1H), 7.53-7.49 (m, 3H), 7.46-7.35 (m, 3H), 7.19 (d, J=4.8 Hz, 1H), 6.86 (d, J=9.1 Hz, 2H), 6.62 (dd, J=3.3, 1.8 Hz, 1H), 3.99 (s, 2H), 3.71 (s, 3H); MS (m/z) 382 [M+H]⁺.

### Example 38

### 4-({2-[2-(4,4-difluoropiperidin-1-yl)-2-oxoethyl]phenyl}ethynyl)-1H-pyrrolo[2,3-b]pyridine

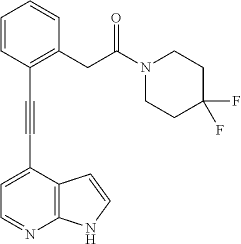

The title compound was obtained in 49% yield from [2-(1H-pyrrolo[2,3-b]pyridin-4-ylethynyl)-phenyl]-acetic acid (example 34) and 4,4-difluoropiperidine hydrochloride following the procedure described for example 35. (HPLC: 99%, RT: 5.96 min) ¹H NMR (DMSO-d₆) δ=11.93 (br s, 1H), 8.24 (d, J=5.1 Hz, 1H), 7.66 (d, J=7.7 Hz, 1H), 7.60 (dd, J=3.3, 2.6 Hz, 1H), 7.45-7.32 (m, 3H), 7.18 (d, J=5.1 Hz, 1H), 6.57 (dd, J=3.7, 1.8 Hz, 1H), 4.06 (s, 2H), 3.69-3.54 (m, 4H), 1.99-1.80 (m, 4H); MS (m/z) 380 [M+H]⁺.

### Example 39

### 2,2,2-Trifluoro-N-[2-(7H-pyrrolo[2,3-d]pyrimidin-4-ylethynyl)-benzyl]-acetamide

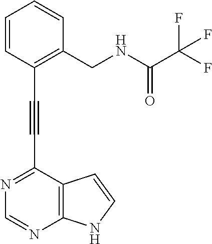

### Intermediate 39.1: 4-Trimethylsilanylethynyl-7H-pyrrolo[2,3-d]pyrimidine

Intermediate 39.1 was obtained in 29% yield from 4-chloro-7H-pyrrolo[2,3-d]pyrimidine and ethynyltrimethylsilane following the procedure described for example 18. (HPLC: 98%, RT: 6.13 min) ¹H NMR (DMSO-d₆) δ=8.72 (s, 1H), 7.68 (dd, J=3.7, 2.2 Hz, 1H), 6.55 (dd, J=3.5, 1.8 Hz, 1H), 0.31 (s, 9H); MS (m/z) 216 [M+H]⁺.

### Intermediate 39.2: 4-Ethynyl-7H-pyrrolo[2,3-d]pyrimidine

Intermediate 39.2 was obtained in 88% yield from intermediate 39.1 following the procedure described for intermediate 10.2. (HPLC: 92%, RT: 3.15 min) ¹H NMR (DMSO-d₆) δ=12.38 (br s, 1H), 8.74 (s, 1H), 7.68 (dd, J=3.3, 2.2 Hz, 1H), 6.59 (dd, J=3.3, 1.8 Hz, 1H), 4.84 (s, 1H); MS (m/z) 144 [M+H]⁺.

### Example 39

### 2,2,2-Trifluoro-N-[2-(7H-pyrrolo[2,3-d]pyrimidin-4-ylethynyl)-benzyl]-acetamide

The title compound was obtained in 31% yield from intermediate 39.2 and 2,2,2-trifluoro-N-(2-iodobenzyl)acetamide following the procedure described for example 10. (HPLC: 99%, RT: 5.64 min) ¹H NMR (DMSO-d₆) δ=12.39 (br s, 1H), 10.14 (t, J=5.5 Hz, 1H), 8.78 (s, 1H), 7.77 (dd, J=7.7, 1.5 Hz, 1H), 7.72 (dd, J=3.3, 2.2 Hz, 1H), 7.55 (td, J=7.7, 1.5 Hz, 1H), 7.45 (td, J=7.7, 1.1 Hz, 1H), 7.39 (d, J=7.3 Hz, 1H), 6.75 (dd, J=3.7, 1.5 Hz, 1H), 4.74 (d, J=5.5 Hz, 2H); MS (m/z) 345 [M+H]⁺.

### Example 40

### Synthesis of: 3-(5,6,7,8-Tetrahydro-[1,8]naphthyridin-4-ylethynyl)-phenylamine

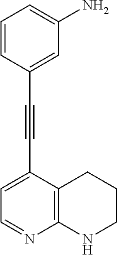

### Intermediate 40.1: 5-Iodoethynyl-1,2,3,4-tetrahydro-[1,8]naphthyridine

Intermediate 40.1 was obtained in 2% yield from 5-chloro-1,2,3,4-tetrahydro-1,8-naphthyridine following the procedure described for intermediate 1.1. ¹H NMR (DMSO-d₆) δ=7.37 (d, J=5.5 Hz, 1H), 6.89 (d, J=5.5 Hz, 1H), 6.64 (br s, 1H), 3.25-3.18 (m, 2H), 2.58 (t, J=6.2 Hz, 2H), 1.82-1.75 (m, 2H); MS (m/z) 261 [M+H]⁺.

### Example 40

### 34,5,6,7,8-Tetrahydro-[1,8]naphthyridin-4-ylethynyl)-phenylamine

The title compound was obtained from intermediate 40.1 and 3-ethynylaniline following the procedure described for example 1. The purified product was dissolved in methanol (2 mL) and the hydrochloric salt was precipitated by addition of a solution 2 N of hydrogen chloride in ether (5 mL) and ether (10 mL). The precipitate was then filtered to afford the title compound (15 mg, 45%) as a beige solid (HPLC: 98%, RT: 2.82 min) ¹H NMR (DMSO-d₆) δ=8.64 (br s, 1H), 7.83 (d, J=6.6 Hz, 1H), 7.39-7.34 (m, 1H), 7.26-7.22 (m, 2H), 7.12 (br d, J=10.3 Hz, 1H), 6.87 (d, J=6.6 Hz, 1H), 3.44 (br s, 2H), 2.93 (t, J=6.2 Hz, 2H), 1.93-1.87 (m, 2H); MS (m/z) 250 [M+H]⁺.

### Example 41

### 4-(phenylethynyl)-5,6,7,8-tetrahydro[1]benzothieno[2,3-d]pyrimidine

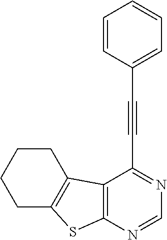

### General Procedure A

Dichloro bis(triphenylphosphine)palladium(II) (17.6 mg; 0.03 mmol; 0.05 equiv.), copper(I) iodide (9.6 mg; 0.05 mmol; 0.10 equiv.) and triethylamine (0.35 ml; 2.5 mmol; 5.0 equiv.) was added to 4-chloro-5,6,7,8-tetrahydro[1]benzothieno[2,3-d]pyrimidine (113.0 mg; 0.50 mmol; 1.00 equiv.) and alkyne (2.51 mmol; 5.00 equiv.) in anhydrous dioxane (2 ml). The reaction mixture was purged with nitrogen gas, capped and heated at 90° C. for 4 h. The reaction mixture was then filtered through a Celite pad, washed with ethyl acetate and concentrated. The crude mixture was purified by flash column chromatography on silica gel to yield the desired product.

4-(phenylethynyl)-5,6,7,8-tetrahydro[1]benzothieno[2,3-d]pyrimidine was synthesized according to general procedure A as a light yellow solid in 72% yield. ¹HNMR (in CDCl₃) δ=1.90-1.97 (m, 4H), 2.86-2.93 (m, 2H), 3.19-3.25 (m, 2H), 7.38-7.43 (m, 3H), 7.61-7.65 (m, 2H), 8.93 (s, 1H). Mass: M+H⁺: 291

### Example 42

### N,N-dimethyl-4-(5,6,7,8-tetrahydro[1]benzothieno[2,3-d]pyrimidin-4-ylethynyl)aniline

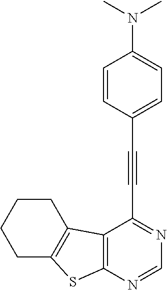

N,N-dimethyl-4-(5,6,7,8-tetrahydro[1]benzothieno[2,3-d]pyrimidin-4-ylethynyl)aniline was synthesized according to general procedure A as a bright yellow solid in 61% yield. ¹HNMR (in DMSO-d₆) δ=1.89 (br, 4H), 2.89 (br, 2H), 2.99 (s, 6H), 3.17 (br, 2H), 6.77 (dt, J=7.6 and 1.8 Hz, 2H), 7.48 (dt, J=7.6 and 1.8 Hz, 2H), 8.87 (s, 1H). Mass: M+H⁺: 334.

### Material and Methods:

Enzyme activity for VEGFR3, VEGFR2 and Flt3 were measured on the Caliper Life Sciences LC3000 (Hopkinton, Mass.). The system utilizes the principle of electroosmotic flow to separate and quantify the amount of phosphorylated-Fluorescein-labelled-peptide (product) from unphosphorylated-Fluorescein-labelled peptide (substrate). The amount of product and substrate are determined by measuring the peak heights from the electropherogram. The enzyme activity is then quantified by dividing the amount of product by the sum of the product and substrate. The inhibitory activity of a sample is measured by comparing the enzyme activity in the presence of sample versus the enzyme activity in the presence of dimethylsufoxide (DMSO). Specific assay conditions for each enzyme are listed below:

VEGFR3 assay: The peptide substrate at 1 uM (FITC-KKKKEEIYFFF-CONH2; synthesized at Tufts University, Boston, Mass.) was incubated with 400 uM of ATP, corresponding to the Michaelis-Menten (Km) constant of the enzyme/substrate reaction, and 0.4 nM of VEGFR3 (Millpore Corp., Cat. No. 14-681) for 90 minutes at RT. After 90 minutes, the reaction was stopped by the addition of 10 mM EDTA. The substrate and product were then separated on the LC3000.

VEGFR2 assay: The peptide substrate at 1 uM (5-FL-EEPLYWSFPAKKK-CONH2; synthesized at Tufts University, Boston, Mass.) was incubated with 160 uM of ATP, corresponding to the Michaelis-Menten (Km) constant of the enzyme/substrate reaction, and 1 nM of VEGFR2 (BPS Bioscience; San Diego, Calif.; Cat. No. 40301) for 90 minutes at RT. After 90 minutes, the reaction was stopped by the addition of 10 mM EDTA. The substrate and product were then separated on the LC3000.

Flt3 assay: The peptide substrate at 1 uM (FITC-AHA-UEAIYAAPFAKKK-CONH2; synthesized at Tufts University, Boston, Mass.) was incubated with 350 uM of ATP, corresponding to the Michaelis-Menten (Km) constant of the enzyme/substrate reaction, and 3 nM of Flt3 (BPS Bioscience; San Diego, Calif.; Cat. No. 40225) for 90 minutes at RT. After 90 minutes, the reaction was stopped by the addition of 10 mM EDTA. The substrate and product were then separated on the LC3000.

TABLE 1
Compounds of the invention include the following and pharmaceutically acceptable
salts thereof:

| ID No. | Structure | VEGFR3 | VEGFR2 | Flt3 |
|---|---|---|---|---|
| 1 | Staurosporine-4 | *** | *** | *** |
| 2 | 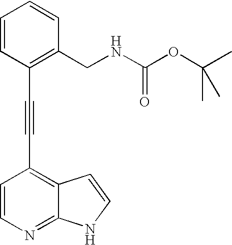 | *** | * | * |
| 3 | 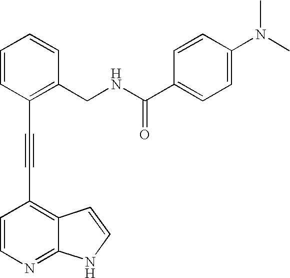 | *** | * | * |
| 4 | 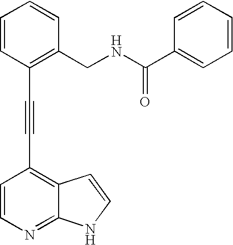 | *** | * | * |
| 5 | 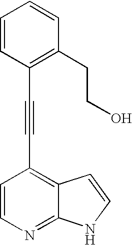 | *** | ** | *** |
| 6 | 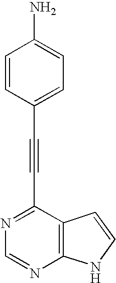 | *** | ** | ** |
| 7 | 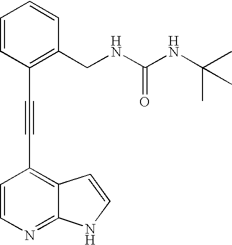 | *** | * | * |
| 8 | 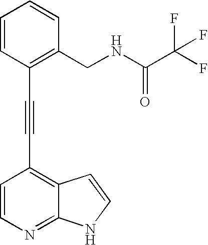 | *** | * | ** |
| 9 | 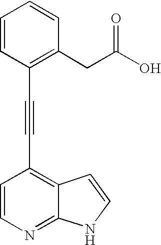 | *** | ** | * |
| 10 | 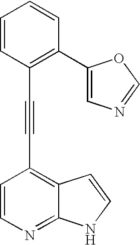 | *** | ** | ** |
| 11 | 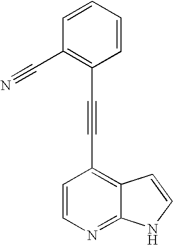 | *** | ** | ** |
| 12 | 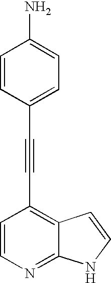 | *** | ** | ** |
| 13 | 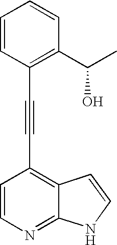 | *** | ** | ** |
| 14 | 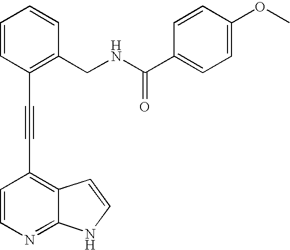 | *** | * | * |
| 15 | 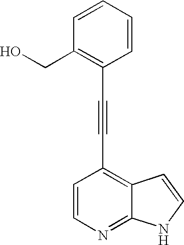 | *** | ** | ** |
| 16 | 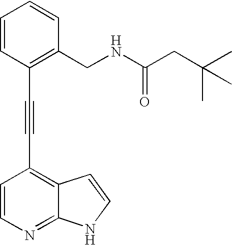 | ** | * | * |
| 17 | 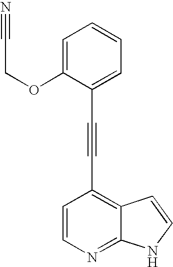 | ** | ** | ** |
| 18 | 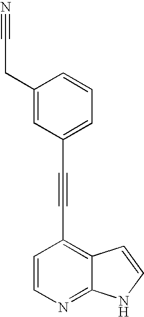 | ** | ** | ** |
| 19 | 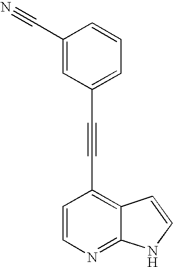 | ** | * | ** |
| 20 | 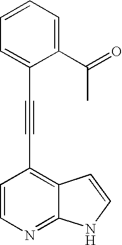 | ** | ** | ** |
| 21 | 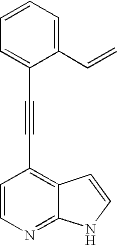 | ** | ** | ** |
| 22 | 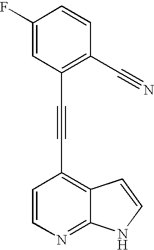 | ** | * | ** |
| 23 | 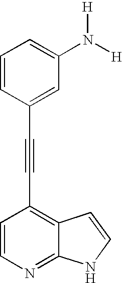 | ** | * | ** |
| 24 | 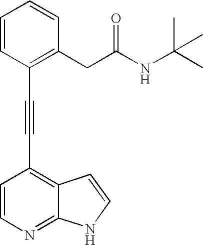 | ** | * | * |
| 25 | 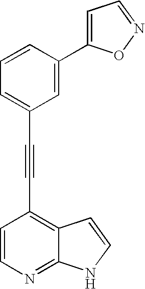 | ** | * | ** |
| 26 | 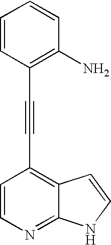 | ** | * | ** |
| 27 | 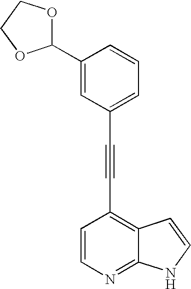 | ** | * | ** |
| 28 | 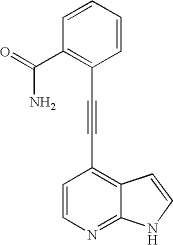 | ** | * | * |
| 29 | 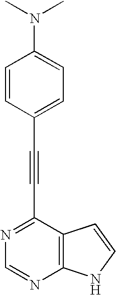 | ** | ** | ** |
| 30 | 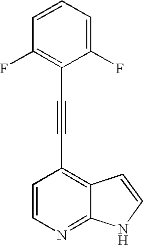 | ** | * | ** |
| 31 | 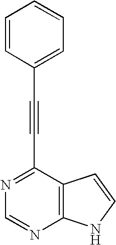 | ** | ** | ** |
| 32 | 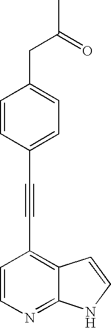 | ** | * | ** |
| 33 | 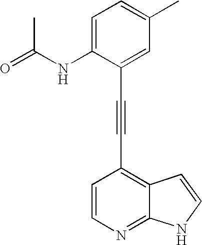 | ** | * | * |
| 34 | 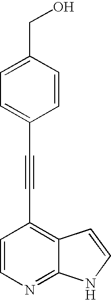 | ** | * | ** |
| 35 | 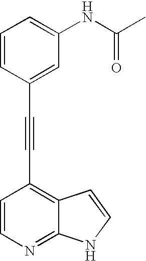 | ** | * | ** |
| 36 | 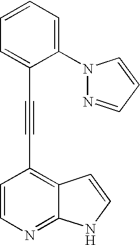 | ** | * | ** |
| 37 | 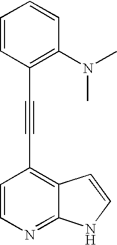 | ** | * | * |
| 38 | 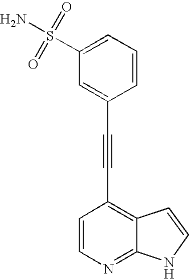 | ** | * | ** |
| 39 | 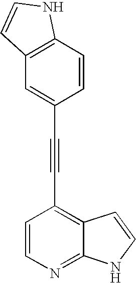 | ** | * | ** |
| 40 | 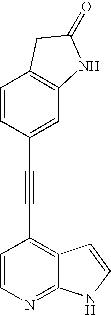 | ** | * | ** |
| 41 | 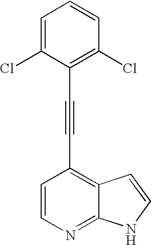 | ** | * | * |
| 42 | 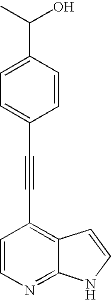 | ** | * | * |
| 43 | 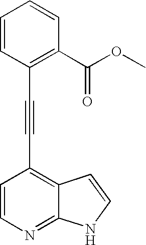 | ** | * | ** |
| 44 | 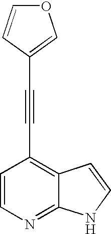 | ** | * | * |
| 45 | 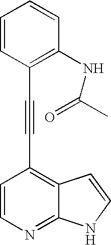 | ** | * | * |
| 46 | 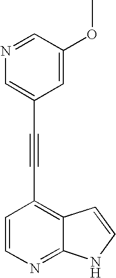 | ** | ** | ** |
| 47 | 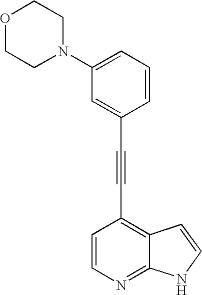 | ** | * | * |
| 48 | 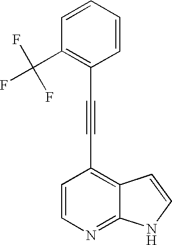 | ** | * | * |
| 49 | 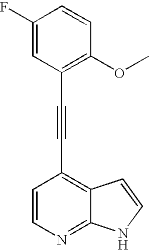 | ** | * | * |
| 50 | 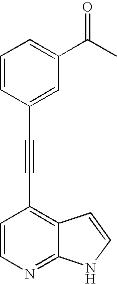 | ** | * | ** |
| 51 | 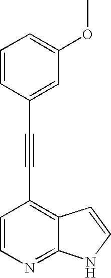 | ** | * | ** |
| 52 | 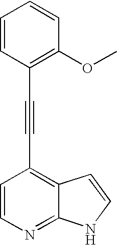 | ** | * | * |
| 53 | 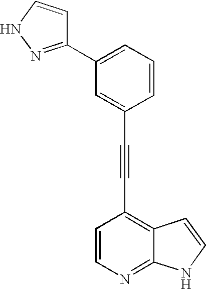 | ** | * | * |
| 54 | 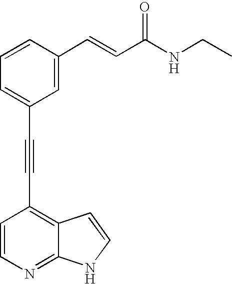 | ** | * | * |
| 55 | 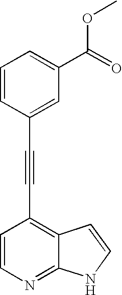 | ** | * | * |
| 56 | 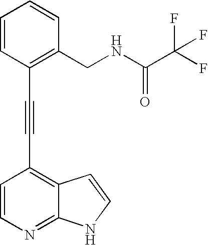 | ** | * | * |
| 57 | 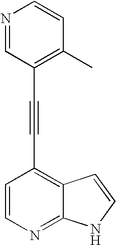 | ** | ** | ** |
| 58 | 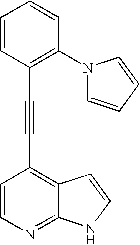 | ** | * | * |
| 59 | 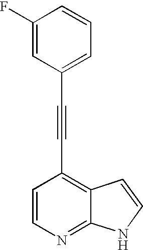 | ** | * | * |
| 60 | 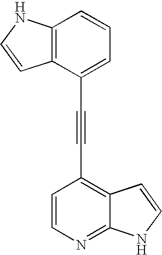 | ** | * | * |
| 61 | 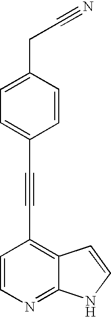 | ** | * | ** |
| 62 | 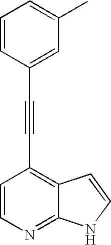 | ** | * | * |
| 63 | 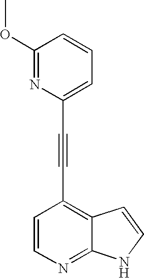 | ** | * | * |
| 64 | 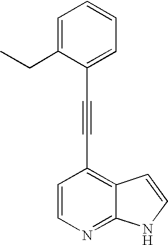 | ** | * | * |
| 65 | 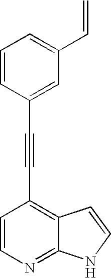 | ** | * | * |
| 66 | 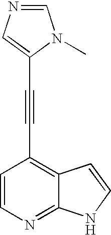 | ** | ** | ** |
| 67 | 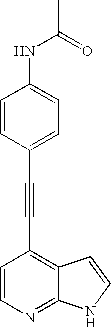 | ** | * | * |
| 68 | 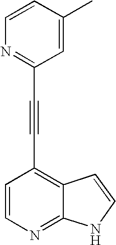 | ** | * | * |
| 69 | 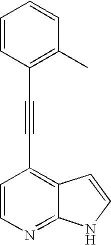 | ** | * | * |
| 70 | 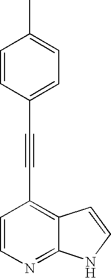 | ** | * | * |
| 71 | 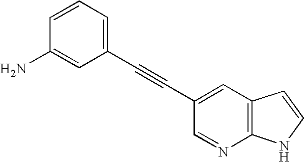 | ** | * | * |
| 72 | 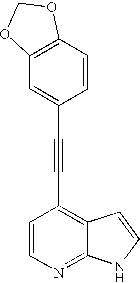 | ** | * | * |
| 73 | 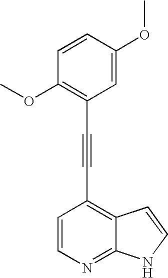 | ** | * | * |
| 74 | 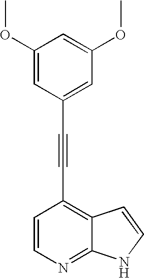 | * | * | * |
| 75 | 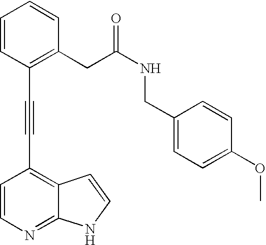 | * | * | * |
| 76 | 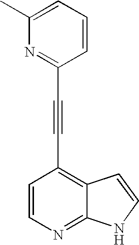 | * | * | * |
| 77 | 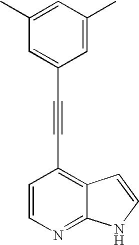 | * | * | * |
| 78 | 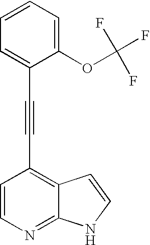 | * | * | * |
| 79 | 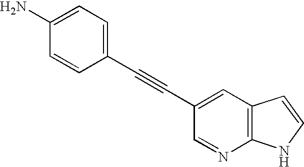 | * | * | ** |
| 80 | 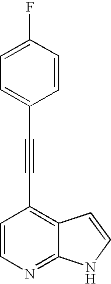 | * | * | * |
| 81 | 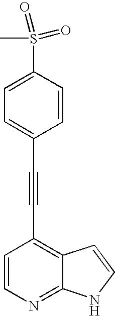 | * | * | * |
| 82 | 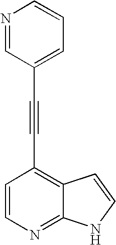 | * | * | * |
| 83 | 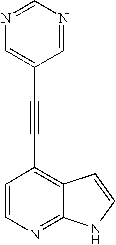 | * | * | * |
| 84 | 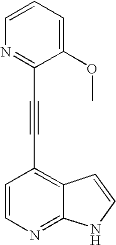 | * | * | * |
| 85 | 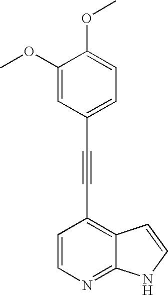 | * | * | * |
| 86 | 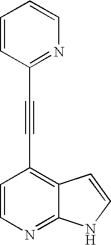 | * | * | * |
| 87 | 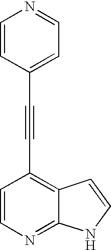 | * | * | * |
| 88 | 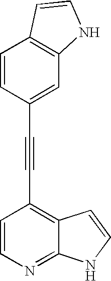 | * | * | * |
| 89 | 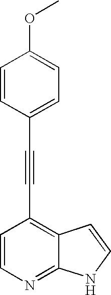 | * | * | * |
| 90 | 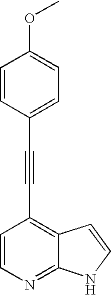 | * | * | * |
| 91 | 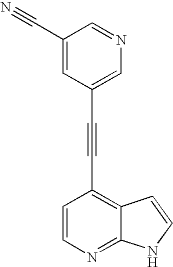 | * | * | * |
| 92 | 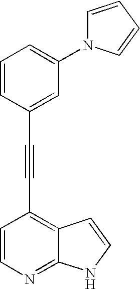 | * | * | * |
| 93 | 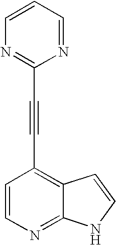 | * | * | * |
| 94 | 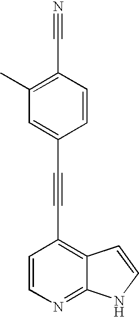 | * | * | * |
| 95 | 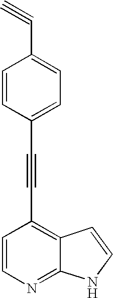 | * | * | * |
| 96 | 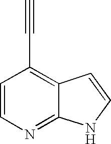 | * | * | * |
| 97 | 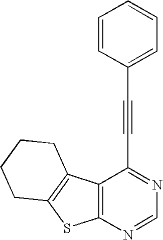 | * | * | * |
| 98 | 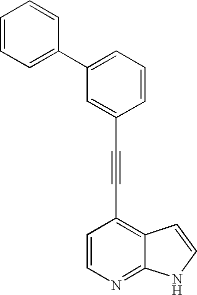 | * | * | * |
| 99 | 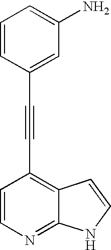 | * | * | * |
| 100 | 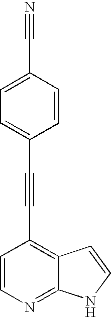 | * | * | * |
| 101 | 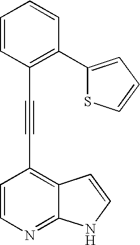 | * | * | * |
| 102 | 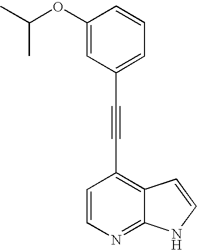 | * | * | * |
| 103 | 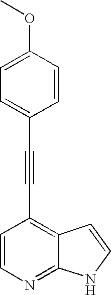 | * | * | * |
| 104 | 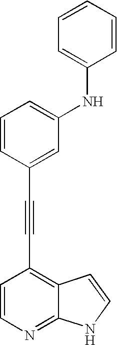 | * | * | * |
| 105 | 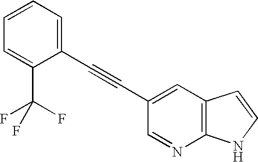 | * | * | * |
| 106 | 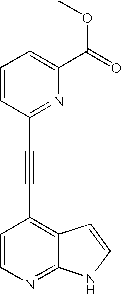 | * | * | * |
| 107 | 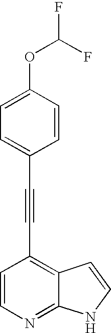 | * | * | * |
| 108 | 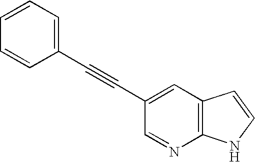 | * | * | * |
| 109 | 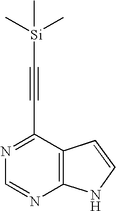 | * | * | * |
| 110 | 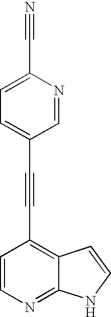 | * | * | * |
| 111 | 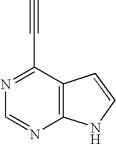 | * | * | * |
| 112 | 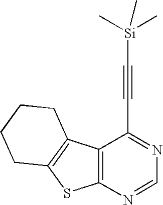 | * | * | * |
| 113 | 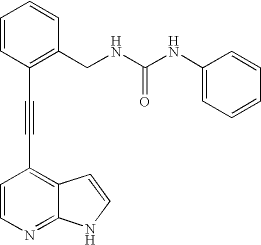 | * | * | * |
| 114 | 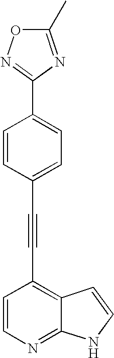 | * | * | * |
| 115 | 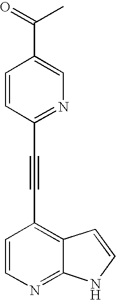 | * | * | * |
| 116 | 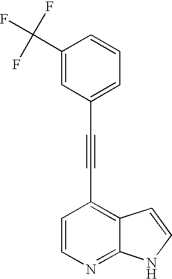 | * | * | * |
| 117 | 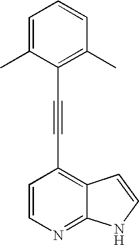 | * | * | * |
| 118 | 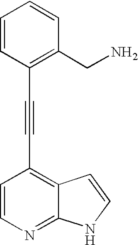 | * | * | * |
| 119 | 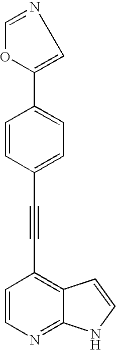 | * | * | * |
| 120 | 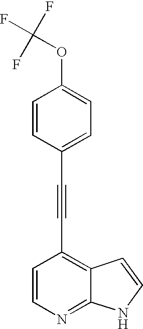 | * | * | * |
| 121 | 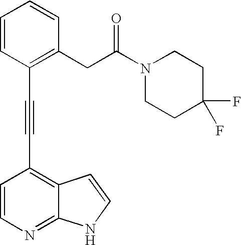 | * | * | * |
| 122 | 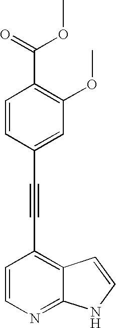 | * | * | * |
| 123 | 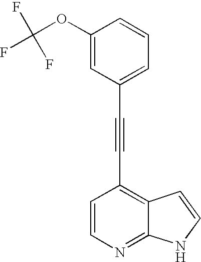 | * | * | * |
| 124 | 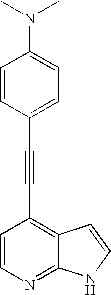 | * | * | * |
| 125 | 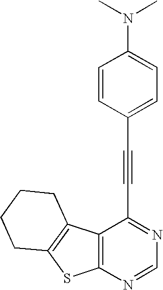 | * | * | * |
| 126 | 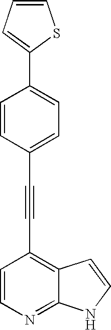 | * | * | * |
| 127 | 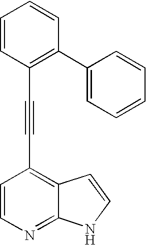 | * | * | * |
| 128 | 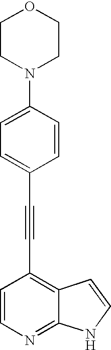 | * | * | * |
| 129 | 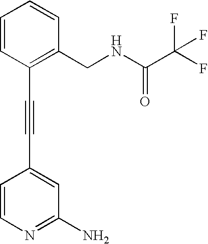 | * | * | * |
| 130 | 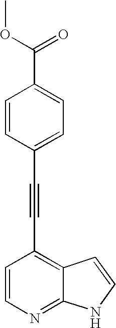 | * | * | * |
| 131 | 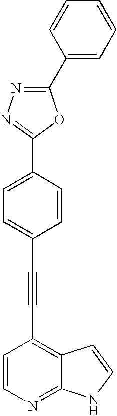 | * | * | * |
| 132 | 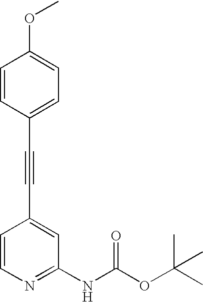 | * | * | * |
| 133 | 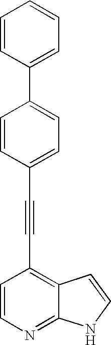 | * | * | * |
| 134 | 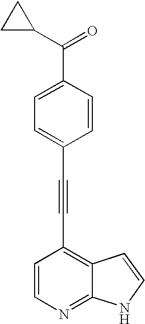 | * | * | * |
| 135 | 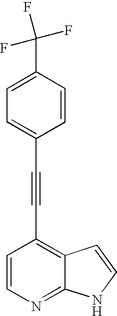 | * | * | * |
| 136 | 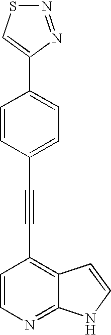 | * | * | * |
| 137 | 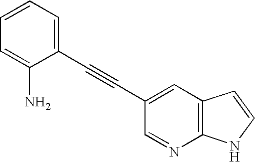 | * | * | * |
| 138 | 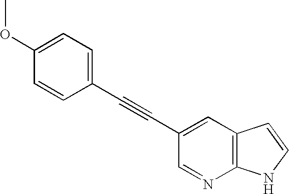 | * | * | * |
| 139 | 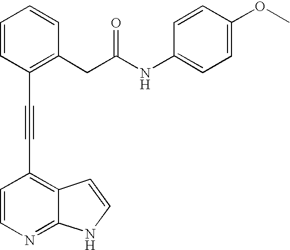 | * | * | * |

Coded Data: </= 100 nm = ***, 100 nM-1000 nM = **, >/= 1000 nM = *

While this invention has been particularly shown and described with references to example embodiments thereof, it will be understood by those skilled in the art that various changes in form and details may be made therein without departing from the scope of the invention encompassed by the appended claims.

1. A compound of the Formula: 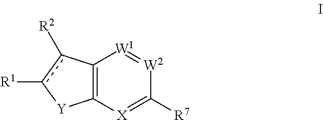
wherein:
W¹ is CR⁸ or N, and W² is —C—C≡C—Ar; or
W¹ is —C—C≡C—Ar, and W² is CR⁸ or N;
Y is —S—, —O—, —NH—, or —NHCH₂—;
X is N or N⁺—O⁻; [symbol]
represents either a single or a double bond;
Ar is aryl, carbocyclyl, heteroaryl or heterocyclyl; wherein the aryl and heteroaryl are optionally and independently substituted with up to 4 groups represented by R³, and wherein the carbocyclyl and heterocyclyl are optionally and independently substituted with up to 4 groups represented by R⁴;
R¹ and R² are independently selected from H, halogen, cyano, (C₁-C₆)alkyl, (C₂-C₆)alkenyl, (C₂-C₆)alkynyl, (C₁-C₆)alkoxy, (C₃-C₈)cycloalkyl, 5-10 membered heterocycloalkyl, —C(O)OR⁵, —C(O)R⁵, —OC(O)R⁵, —C(O)NR⁵R⁶, —NR⁵C(O)NR⁵R⁶, —NR⁵C(O)OR⁵, —NR⁵C(O)R⁵, —NR⁵C(S)NR⁵R⁶, —S(O)ₚNR⁵R⁶, —NR⁵R⁶, —S(O)ₚR⁵, —NR⁵S(O)ₚR⁵, wherein said alkyl, alkenyl, alkynyl, alkoxy, cycloalkyl, and heterocycloalkyl are each substituted or unsubstituted; or
R¹ and R² can be taken together with their intervening atoms to form a (C₅-C₆)cycloalkyl ring which is substituted or unsubstituted; and
p is an integer from 0 to 2;
each R³ is independently:
i) halogen, —X₁—OH, —X₁—CN, —X₁—OR¹⁰, —X₁—CO₂R¹⁰, —X₁—NR¹⁰C(O)N(R¹⁰)₂, —X₁—NR¹⁰C(S)N(R¹⁰)₂, —X₁NR¹⁰CO₂R¹⁰, —X₁COR¹⁰, —X₁N(R¹⁰)₂, —X₁N⁺ (R¹⁰)₂, —X₁—OCOR¹⁰, —X₁SO₂N(R¹⁰)₂; —₁—S(O)_{N}R¹⁰; —X₁NR¹⁰S(O)_{N}R¹⁰, —X₁—NR¹⁰COR¹⁰, —X₁—OC(O)N(R¹⁰)₂.—X₁—CO(R¹⁰)₂, or —X₁—NR¹⁰CO₂R²⁰; or
ii) (C₁-C₆)alkyl, (C₁-C₆)haloalkyl, (C₂-C₆)alkenyl, (C₂-C₆)haloalkenyl, (C₂-C₆)alkynyl or (C₂-C₆)haloalkynyl; or
iii) aryl, aralkyl, aryloxy, heteroaryl, heteroaralkyl, or heteroaryloxy, each optionally and independently substituted with up to 3 groups selected from halogen, —CN, —OH, —NH₂, —NH(C₁-C₆)alkyl, —N((C₁-C₆)alkyl)₂, (C₁-C₆)alkyl, halo(C₁-C₆)alkyl, (C₁-C₆)alkoxy, halo(C₁-C₆)alkoxy, (C₁-C₆)alkoxycarbonyl, —CONH₂, —CONH(C₁-C₆)alkyl, —CON((C₁-C₆)alkyl)₂, —CO(C₁-C₆)alkyl or —CO₂H; or
iv) carbocyclyl or heterocyclyl, each optionally and independently substituted with up to 3 groups selected from halogen, —CN, —OH, —NH₂, —NH(C₁-C₆)alkyl, —N((C₁-C₆)alkyl)₂, (C₁-C₆)alkyl, halo(C₁-C₆)alkyl, (C₁-C₆)alkoxy, halo(C₁-C₆)alkoxy, (C₁-C₆)alkoxycarbonyl, —CONH₂, —CONH(C₁-C₆)alkyl, —CON((C₁-C₆)alkyl)₂, —CO(C₁-C₆)alkyl, —CO₂H, aryl, heteroaryl, oxo and thioxo;
each X₁ is independently a covalent bond, a (C₁-C₆)alkylene, (C₁-C₆)alkenylene or (C₁-C₆)alkynylene;
each R⁴ is independently a group represented by R³, oxo or thioxo;
n is an integer from 0 to 2;
each R⁵ and R⁶ are independently selected from H, (C₁-C₄)alkyl, (C₃-C₈)cycloalkyl or phenyl; wherein said alkyl, cycloalkyl and phenyl are optionally and independently substituted with halogen, —CN, —OH, —NH₂, —OCF₃, —OMe, or (C₁-C₃)alkyl;
R⁷ and R⁸ are independently H, halogen, cyano, (C₁-C₆)alkyl, (C₂-C₆)alkenyl, (C₂-C₆)alkynyl, —OR⁵, (C₃-C₈)cycloalkyl, 5-10 membered heterocycloalkyl, —C(O)OR⁵, —C(O)R⁵, —OC(O)R⁵, —C(O)NR⁵R⁶, —NR⁵C(O)NR⁵R⁶, —NR⁵C(O)OR⁵, —NR⁵C(O)R⁵, —NR⁵C(S)NR⁵R⁶, —S(O)ₚNR⁵R⁶, —NR⁵R⁶, —SR⁵, —NR⁵S(O)ₚR⁵, wherein said alkyl, alkenyl, alkynyl, cycloalkyl, and heterocycloalkyl are each substituted or unsubstituted; and
each R¹⁰ is independently H, (C₁-C₆)alkyl, (C₂-C₆)alkenyl, (C₂-C₆)alkynyl, (C₃-C₈)cycloalkyl, 5-10 membered heterocycloalkyl, aryl, aralkyl, heteroaryl, or heteroaralkyl; wherein said alkyl, alkenyl, alkynyl, alkoxy, cycloalkyl, heterocycloalkyl, aryl, aralkyl, heteroaryl, and heteroaralkyl are optionally and independently substituted with halogen, —CN, —OH, —NH₂, —NH(C₁-C₃)alkyl, —N((C₁-C₃)alkyl)₂, —CONH₂, —CONH(C₁-C₃)alkyl, —CON((C₁-C₃)alkyl)₂, —CO(C₁-C₃)alkyl, —CO₂H, (C₁-C₃)alkyl, (C₁-C₃)haloalkyl, (C₁-C₃)alkoxy, (C₁-C₃)haloalkyl, (C₁-C₃)haloalkoxy, (C₁-C₃)alkyoxycarbonyl, (C₃-C₇)cycloalkyl, or phenyl;
or a pharmaceutically acceptable salt thereof.

2. The compound according to claim 1, wherein:
R¹ and R² are independently selected from H, halogen, cyano, (C₁-C₆)alkyl, (C₂-C₆)alkenyl, (C₂-C₆)alkynyl, (C₁-C₆)alkoxy, (C₃-C₈)cycloalkyl, 5-10 membered heterocycloalkyl, —C(O)OR⁵, —C(O)R⁵, —OC(O)R⁵, —C(O)NR⁵R⁶, —NR⁵C(O)NR⁵R⁶, —NR⁵C(O)OR⁵, —NR⁵C(O)R⁵, —NR⁵C(S)NR⁵R⁶, —S(O)ₚNR⁵R⁶, —NR⁵R⁶, —S(O)ₚR⁵, —NR⁵S(O)ₚR⁵; or
R¹ and R² can be taken together with their intervening atoms to form a (C₅-C₆)cycloalkyl ring; and
wherein said alkyl, alkenyl, alkynyl, alkoxy, cycloalkyl, heterocycloalkyl or (C₅-C₆)cycloalkyl ring represented by R¹ and/or R² are optionally and independently substituted with halogen, —CN, —OH, —NH₂, —NH(C₁-C₆)alkyl, —N((C₁-C₆)alkyl)₂, (C₁-C₆)alkoxy, (C₁-C₆)alkoxycarbonyl, —CONH₂, —OCONH₂, —NHCONH₂, —N(C₁-C₆)alkylCONH₂, —N(C₁-C₆)alkylCONH(C₁-C₆)alkyl, —NHCONH(C₁-C₆)alkyl, —NHCON((C₁-C₆)alkyl)₂, —N(C₁-C₆)alkylCON((C₁-C₆)alkyl)₂, —NHC(S)NH₂, —N(C₁-C₆)alkylC(S)NH₂, —N(C₁-C₆)alkylC(S)NH(C₁-C₆)alkyl, —NHC(S)NH(C₁-C₆)alkyl, —NHC(S)N((C₁-C₆)alkyl)₂, —N(C₁-C₆)alkylC(S)N((C₁-C₆)alkyl)₂, —CONH(C₁-C₆)alkyl, —OCONH(C₁-C₆)alkyl —CON((C₁-C₆)alkyl)₂, —C(S)(C₁-C₆)alkyl, —S(O)ₚ(C₁-C₆)alkyl, —S(O)ₚNH₂, —S(O)ₚNH(C₁-C₆)alkyl, —S(O)ₚN((C₁-C₆)alkyl)₂, —CO(C₁-C₆)alkyl, —OCO(C₁-C₆)alkyl, —C(O)O(C₁-C₆)alkyl, —OC(O)O(C₁-C₆)alkyl, —C(O)H or —CO₂H; and
R⁷ and R⁸ are independently H, halogen, cyano, (C₁-C₆)alkyl, (C₂-C₆)alkenyl, (C₂-C₆)alkynyl, —OR⁵, (C₃-C₈)cycloalkyl, 5-10 membered heterocycloalkyl, —C(O)OR⁵, —C(O)R⁵, —OC(O)R⁵, —C(O)NR⁵R⁶, —NR⁵C(O)NR⁵R⁶, —NR⁵C(O)OR⁵, —NR⁵C(O)R⁵, —NR⁵C(S)NR⁵R⁶, —S(O)ₚNR⁵R⁶, —NR⁵R⁶, —SR⁵, —NR⁵S(O)ₚR⁵, wherein said alkyl, alkenyl, alkynyl, cycloalkyl, and heterocycloalkyl represented by R⁷ and R⁸ are each optionally substituted with halogen, —CN, —OH, —NH₂, —NH(C₁-C₆)alkyl, —N((C₁-C₆)alkyl)₂, (C₁-C₆)alkoxy, (C₁-C₆)alkoxycarbonyl, —CONH₂, —OCONH₂, —NHCONH₂, —N(C₁-C₆)alkylCONH₂, —N(C₁-C₆)alkylCONH(C₁-C₆)alkyl, —NHCONH(C₁-C₆)alkyl, —NHCON((C₁-C₆)alkyl)₂, —N(C₁-C₆)alkylCON((C₁-C₆)alkyl)₂, —NHC(S)NH₂, —N(C₁-C₆)alkylC(S)NH₂, —N(C₁-C₆)alkylC(S)NH(C₁-C₆)alkyl, —NHC(S)NH(C₁-C₆)alkyl, —NHC(S)N((C₁-C₆)alkyl)₂, —N(C₁-C₆)alkylC(S)N((C₁-C₆)alkyl)₂, —CONH(C₁-C₆)alkyl, —OCONH(C₁-C₆)alkyl —CON((C₁-C₆)alkyl)₂, —C(S)(C₁-C₆)alkyl, —S(O)ₚ(C₁-C₆)alkyl, —S(O)ₚNH₂, —S(O)ₚNH(C₁-C₆)alkyl, —S(O)ₚN((C₁-C₆)alkyl)₂, —CO(C₁-C₆)alkyl, —OCO(C₁-C₆)alkyl, —C(O)O(C₁-C₆)alkyl, —OC(O)O(C₁-C₆)alkyl, —C(O)H or —CO₂H;
or a pharmaceutically acceptable salt thereof.

3. The compound according to claim 1 or 2, according to the formula: 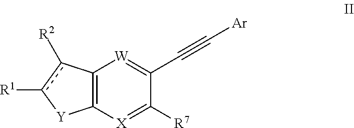
wherein W is N or CR⁸;
or a pharmaceutically acceptable salt thereof.

4. (canceled)

5. The compound of claim 1, according to the Formula: 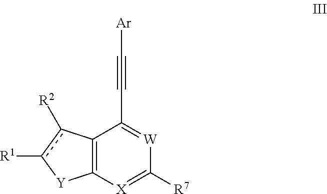
wherein W is N or CR⁸;
or a pharmaceutically acceptable salt thereof.

6-24. (canceled)

25. The compound claim 1, 2, 3 or 5, wherein:
each R³ is independently:
i) halogen, —X₁—OH, —X₁—CN, —X₁—CO₂R¹⁰, —X₁—OR¹⁰, —X₁—NR¹⁰C(O)N(R¹⁰)₂, —X₁—NR¹⁰C(S)N(R¹⁰)₂, —X₁COR¹⁰, —X₁—N(R¹⁰)₃, —X₁N(R¹⁰)₃, —X₁OCOR¹⁰, —X₁—SO₂N(R¹⁰)₂, —X₁—S(O)—R¹⁰, —X₁—NR¹⁰S(O)ₙR¹⁰, —X₁—NR¹⁰COR¹⁰, —X₁—CON(R¹⁰)₂, or —X₁—NR¹⁰CO₂R¹⁰;
ii) (C₁-C₆)alkyl, (C₁-C₆)haloalkyl, (C₂-C₆)alkenyl, (C₂-C₆)haloalkenyl, (C₂-C₆)alkynyl or (C₂-C₆)haloalkynyl; or
iii) phenyl, thienyl, oxazolyl, isooxazolyl, oxadiazolyl, thiadiazolyl, thiazolyl, pyridyl, pyrazolyl, or pyrrolyl, each optionally and independently substituted with up to 2 groups selected from halogen, —CN, —OH, —NH₂, (C₁-C₃)alkyl, halo(C₁-C₃)alkyl, phenyl [optionally substituted with halogen, (C₁-C₃)alkyl, (C₁-C₃)alkoxy, (C₁-C₃)haloalkyl, (C₁-C₃)haloalkoxy, —CN or —NO₂], (C₁-C₃)alkoxy, halo(C₁-C₃)alkoxy, —CO₂(C₁-C₃)alkyl, —CONH₂, —CONH(C₁-C₃)alkyl, —CO(C₁-C₃)alkyl or —CO₂H; or
iv) 1,3-dioxolanyl, 1,3-dioxanyl, (C₃-C₆)cycloalkyl, piperidinyl or morpholinyl, each optionally and independently substituted with up to 2 groups selected from halogen, —OH, —NH₂, —O(C₁-C₃)alkyl, (C₁-C₃)alkyl, phenyl, —CO₂H, oxo and thioxo;
X₁ is a covalent bond or (C₁-C₂)alkylene;
each R⁴ is independently halogen, —OH, —NH₂, —O(C₁-C₃)alkyl, (C₁-C₃)alkyl, phenyl, —CO₂H, oxo or thioxo;
n is an integer from 0 to 2;
R⁷ and R⁸ are independently H, halogen or (C₁-C₆)alkyl, wherein the alkyl is optionally substituted by halogen, —CN, —OH, —NH₂, —NH(C₁-C₆)alkyl, —N((C₁-C₆)alkyl)₂, (C₁-C₆)alkoxy, (C₁-C₆)alkoxycarbonyl, —CONH₂, —CONH(C₁-C₆)alkyl, —CON((C₁-C₆)alkyl)₂, —CO(C₁-C₆)alkyl or —CO₂H; and
each R¹⁰ is independently H, (C₁-C₆)alkyl, (C₃-C₆)cycloalkyl, piperidinyl, morpholinyl, benzyl or phenyl; wherein the alkyl, cycloalkyl, piperidinyl, morpholinyl, benzyl and phenyl groups represented by R¹⁰ are optionally and independently substituted with halogen, —CN, —OH, —NH₂, —NH(C₁-C₃)alkyl, —N((C₁-C₃)alkyl)₂, —COMe, —CO₂H, (C₁-C₃)alkyl, halo(C₁-C₃)alkyl, (C₁-C₃)alkoxy or halo(C₁-C₃)alkoxy.

26-28. (canceled)

29. A compound selected from the group consisting of:
3-((1H-pyrrolo[2,3-b]pyridin-4-yl)ethynyl)aniline;
1-(2-((1H-pyrrolo[2,3-b]pyridin-4-yl)ethynyl)phenyl)ethanone;
4((3-methoxyphenyl)ethynyl)-1H-pyrrolo[2,3-b]pyridine;
1-(3-((1H-pyrrolo[2,3-b]pyridin-4-yl)ethynyl)phenyl)ethanone;
4-(m-tolylethynyl)-1H-pyrrolo[2,3-b]pyridine;
4-(biphenyl-4-ylethynyl)-1H-pyrrolo[2,3-b]pyridine;
N-(2-((1H-pyrrolo[2,3-b]pyridin-4-yl(ethynyl)phenyl)acetamide;
4-((3-fluorophenyl)ethynyl)pyridin-2-amine;
4((4-fluorophenyl)ethynyl)-1H-pyrrolo[2,3-b]pyridine;
4((3-vinylphenyl)ethynyl)-1H-pyrrolo[2,3-b]pyridine;
4-(pyridin-3-ylethynyl)-1H-pyrrolo[2,3-b]pyridine;
4((6-methoxypyridin-3-yl)ethynyl)-1H-pyrrolo[2,3-b]pyridine;
4-((1H-pyrrolo[2,3-b]pyridin-4-yl)ethynyl)aniline;
3-((1H-pyrrolo[2,3-b]pyridin-4-yl)ethynyl)aniline; 44(2-(trifluoromethyl)phenyl)ethynyl)-1H-pyrrolo[2,3-b]pyridine;
4((2-methoxyphenyl)ethynyl)-1H-pyrrolo[2,3-b]pyridine;
44(3-(trifluoromethyl)phenyl)ethynyl)-1H-pyrrolo[2,3-b]pyridine;
4((2-vinylphenyl)ethynyl)-1H-pyrrolo[2,3-b]pyridine;
4((2-ethylphenyl)ethynyl)-1H-pyrrolo[2,3-b]pyridine;
4-(biphenyl-3-ylethynyl)-1H-pyrrolo[2,3-b]pyridine;
N-(4-((1H-pyrrolo[2,3-b]pyridin-4-yl)ethynyl)phenyl)acetamide;
4((4-vinylphenyl)ethynyl)-1H-pyrrolo[2,3-b]pyridine;
2-(3-((1H-pyrrolo[2,3-b]pyridin-4-yl)ethynyl)phenyl)acetonitrile;
2-(4-((1H-pyrrolo[2,3-b]pyridin-4-yl)ethynyl)phenyl)acetonitrile;
2-((1H-pyrrolo[2,3-b]pyridin-4-yl)ethynyl)benzamide;
4((5-fluoro-2-methoxyphenyl)ethynyl)-1H-pyrrolo[2,3-b]pyridine;
methyl 3-((1H-pyrrolo[2,3-b]pyridin-4-yl)ethynyl)benzoate;
4((3-(trifluoromethoxy)phenyl)ethynyl)-1H-pyrrolo[2,3-b]pyridine;
4((4-(trifluoromethoxy)phenyl)ethynyl)-1H-pyrrolo[2,3-b]pyridine;
2-((1H-pyrrolo[2,3-b]pyridin-4-yl)ethynyl)benzenesulfonamide;
4((4-(difluoromethoxy)phenyl)ethynyl)-1H-pyrrolo[2,3-b]pyridine;
(E)-3-(3-((1H-pyrrolo[2,3-b]pyridin-4-yl)ethynyl)phenyl)-N-ethylacrylamide;
(2-((1H-pyrrolo[2,3-b]pyridin-4-yl)ethynyl)phenyl)methanol;
4((3,5-dimethoxyphenyl)ethynyl)-1H-pyrrolo[2,3-b]pyridine;
5-((1H-pyrrolo[2,3-b]pyridin-4-yl)ethynyl)picolinonitrile;
4((5-methoxypyridin-3-yl)ethynyl)-1H-pyrrolo[2,3-b]pyridine;
2-((1H-pyrrolo[2,3-b]pyridin-4-yl)ethynyl)benzonitrile;
3-((1H-pyrrolo[2,3-b]pyridin-4-yl)ethynyl)benzonitrile;
4-((1H-pyrrolo[2,3-b]pyridin-4-yl)ethynyl)benzonitrile;
4((4-(methylsulfonyl)phenyl)ethynyl)-1H-pyrrolo[2,3-b]pyridine;
4-((1H-pyrrolo[2,3-b]pyridin-4-yl)ethynyl)-2-methylbenzonitrile;
3-((1H-pyrrolo[2,3-b]pyridin-4-yl)ethynyl)benzenesulfonamide;
4((2-(trifluoromethoxy)phenyl)ethynyl)-1H-pyrrolo[2,3-b]pyridine;
4-(pyrimidin-5-ylethynyl)-1H-pyrrolo[2,3-b]pyridine;
4((4-methylpyridin-3-yl)ethynyl)-1H-pyrrolo[2,3-b]pyridine;
methyl 6-((1H-pyrrolo[2,3-b]pyridin-4-yl)ethynyl)picolinate;
4-(benzo[d][1,3]-dioxol-5-ylethynyl)-1H-pyrrolo[2,3-b]pyridine;
4-((1H-indol-5-yl)ethynyl)-1H-pyrrolo[2,3-b]pyridine;
methyl 2-((1H-pyrrolo[2,3-b]pyridin-4-yl)ethynyl)benzoate;
(4-((1H-pyrrolo[2,3-b]pyridin-4-yl)ethynyl)phenyl)(cyclopropyl)methanone;
3-(4-((1H-pyrrolo[2,3-b]pyridin-4-yl)ethynyl)phenyl)-5-methyl-1,2,4-oxadiazole;
2-(4-((1H-pyrrolo[2,3-b]pyridin-4-yl)ethynyl)phenyl)-5-phenyl-1,3,4-oxadiazole;
methyl 4-((1H-pyrrolo[2,3-b]pyridin-4-yl)ethynyl)-2-methoxybenzoate;
4-(o-tolylethynyl)-1H-pyrrolo[2,3-b]pyridine;
4((3,5-dimethylphenyl)ethynyl)-1H-pyrrolo[2,3-b]pyridine;
4((4-(trifluoromethyl)phenyl)ethynyl)-1H-pyrrolo[2,3-b]pyridine;
(4-((1H-pyrrolo[2,3-b]pyridin-4-yl)ethynyl)phenyl)methanol;
4((3,4-dimethoxyphenyl)ethynyl)-1H-pyrrolo[2,3-b]pyridine;
4((2,5-dimethoxyphenyl)ethynyl)-1H-pyrrolo[2,3-b]pyridine;
methyl 4-((1H-pyrrolo[2,3-b]pyridin-4-yl)ethynyl)benzoate;
4-(4′-((1H-pyrrolo[2,3-b]pyridin-4-yl)ethynyl)biphenyl-4-yl)-1,2,3-thiadiazole;
4-((1H-indol-6-yl)ethynyl)-1H-pyrrolo[2,3-b]pyridine;
4-((1H-indol-4-yl)ethynyl)-1H-pyrrolo[2,3-b]pyridine;
2-(2-((1H-pyrrolo[2,3-b]pyridin-4-yl)ethynyl)phenoxy)acetonitrile;
4-((3-(pyrrolidin-1-yl)phenyl)ethynyl)-1H-pyrrolo[2,3-b]pyridine;
5-(4-((1H-pyrrolo[2,3-b]pyridin-4-yl)ethynyl)phenyl)oxazole;
4-(3-((1H-pyrrolo[2,3-b]pyridin-4-yl)ethynyl)phenyl)morpholine;
4-((2-(1H-pyrazol-1-yl)phenyl)ethynyl)-1H-pyrrolo[2,3-b]pyridine;
4-((2-(1H-pyrazol-1-yl)phenyl)ethynyl)-1H-pyrrolo[2,3-b]pyridine;
4-((3-(1H-pyrazol-1-yl)phenyl)ethynyl)-1H-pyrrolo[2,3-b]pyridine;
4-(biphenyl-2-ylethynyl)-1H-pyrrolo[2,3-b]pyridine;
4((4-(thiophen-2-yl)phenyl)ethynyl)-1H-pyrrolo[2,3-b]pyridine;
4-(4-((1H-pyrrolo[2,3-b]pyridin-4-yl)ethynyl)phenyl)morpholine;
N-(2-((1H-pyrrolo[2,3-b]pyridin-4-yl)ethynyl)-4-methylphenyl)acetamide;
4-(p-tolylethynyl)-1H-pyrrolo[2,3-b]pyridine;
1-(4-((1H-pyrrolo[2,3-b]pyridin-4-yl)ethynyl)phenyl)ethanol;
1-(4-((1H-pyrrolo[2,3-b]pyridin-4-yl)ethynyl)phenyl)propan-2-one;
4-((3-(1H-pyrazol-3-yl)phenyl)ethynyl)-1H-pyrrolo[2,3-b]pyridine;
4((3-isopropoxyphenyl)ethynyl)-1H-pyrrolo[2,3-b]pyridine;
4-((3-(1,3-dioxolan-2-yl)phenyl)ethynyl)-1H-pyrrolo[2,3-b]pyridine;
4((6-methylpyridin-2-yl)ethynyl)-1H-pyrrolo[2,3-b]pyridine;
4((6-methoxypyridin-2-yl)ethynyl)-1H-pyrrolo[2,3-b]pyridine;
1-(6-((1H-pyrrolo[2,3-b]pyridin-4-yl)ethynyl)pyridin-3-yl)ethanone;
4-((1-methyl-1H-imidazol-5-yl)ethynyl)-1H-pyrrolo[2,3-b]pyridine;
44(2,6-dimethylphenyl)ethynyl)-1H-pyrrolo[2,3-b]pyridine;
44(2,6-difluorophenyl)ethynyl)-1H-pyrrolo[2,3-b]pyridine;
5-(3-((1H-pyrrolo[2,3-b]pyridin-4-yl)ethynyl)phenyl)isoxazole;
4-((2-(1H-pyrrol-1-yl)phenyl)ethynyl)-1H-pyrrolo[2,3-b]pyridine;
2-((1H-pyrrolo[2,3-b]pyridin-4-yl)ethynyl)-N,N-dimethylaniline;
3-((1H-pyrrolo[2,3-b]pyridin-4-yl)ethynyl)-N-phenylaniline;
6-((1H-pyrrolo[2,3-b]pyridin-4-yl)ethynyl)indolin-2-one;
4-(pyrimidin-2-ylethynyl)-1H-pyrrolo[2,3-b]pyridine;
4((4-methylpyridin-2-yl)ethynyl)-1H-pyrrolo[2,3-b]pyridine;
5-((1H-pyrrolo[2,3-b]pyridin-4-yl)ethynyl)nicotinonitrile;
4((3-methoxypyridin-2-yl)ethynyl)-1H-pyrrolo[2,3-b]pyridine;
4-(furan-3-ylethynyl)-1H-pyrrolo[2,3-b]pyridine;
4-(phenylethynyl)-1H-pyrrolo[2,3-b]pyridine;
4((3-chlorophenyl)ethynyl)-1H-pyrrolo[2,3-b]pyridine;
4-(pyridin-2-ylethynyl)-1H-pyrrolo[2,3-b]pyridine;
4((4-methoxyphenyl)ethynyl)-1H-pyrrolo[2,3-b]pyridine;
4((2,4-difluorophenyl)ethynyl)-1H-pyrrolo[2,3-b]pyridine;
2-((1H-pyrrolo[2,3-b]pyridin-4-yl)ethynyl)-4-fluorobenzonitrile;
4-(pyridin-4-ylethynyl)-1H-pyrrolo[2,3-b]pyridine;
2-(2-((1H-pyrrolo[2,3-b]pyridin-4-yl)ethynyl)phenyl)ethanol;
tert-butyl 2-((1H-pyrrolo[2,3-b]pyridin-4-yl)ethynyl)benzylcarbamate;
(2-((1H-pyrrolo[2,3-b]pyridin-4-yl)ethynyl)phenyl)methanamine;
44(2,6-dichlorophenyl)ethynyl)-1H-pyrrolo[2,3-b]pyridine;
(S)-1-(2-((1H-pyrrolo[2,3-b]pyridin-4-yl)ethynyl)phenyl)ethanol;
N-(3-((1H-pyrrolo[2,3-b]pyridin-4-yl)ethynyl)phenyl)acetamide;
4((2-(thiophen-2-yl)phenyl)ethynyl)-1H-pyrrolo[2,3-b]pyridine;
5-(2-((1H-pyrrolo[2,3-b]pyridin-4-yl)ethynyl)phenyl)oxazole;
5-(phenylethynyl)-1H-pyrrolo[2,3-b]pyridine;
4-((1H-pyrrolo[2,3-b]pyridin-4-yl)ethynyl)-N,N-dimethylaniline;
4-((1H-pyrrolo[2,3-b]pyridin-5-yl)ethynyl)aniline;
4-((1H-pyrrolo[2,3-b]pyridin-4-yl)ethynyl)-N,N-dimethylaniline;
3-((1H-pyrrolo[2,3-b]pyridin-5-yl)ethynyl)aniline; 5((2-(trifluoromethyl)phenyl)ethynyl)-1H-pyrrolo[2,3-b]pyridine;
4-(phenylethynyl)-7H-pyrrolo[2,3-d]pyrimidine;
5((4-methoxyphenyl)ethynyl)-1H-pyrrolo[2,3-b]pyridine;
4((7H-pyrrolo[2,3-d]pyrimidin-4-yl)ethynyl)aniline;
4((7H-pyrrolo[2,3-d]pyrimidin-4-yl)ethynyl)-N,N-dimethylaniline;
4-(phenylethynyl)-5,6,7,8-tetrahydro-[1]-benzothieno[2,3-d]pyrimidine;
N,N-dimethyl-4-(5,6,7,8-tetrahydro-[1 ]-benzothieno[2,3-d]pyrimidin-4-ylethynyl)aniline;
2-(2-((1H-pyrrolo[2,3-b]pyridin-4-yl)ethynyl)phenyl)acetic acid;
N-(2-((1H-pyrrolo[2,3-b]pyridin-4-yl)ethynyl)benzyl)-3,3-dimethylbutanamide;
N-(2-((1H-pyrrolo[2,3-b]pyridin-4-yl)ethynyl)benzyl)benzamide;
1-(2-((1H-pyrrolo[2,3-b]pyridin-4-yl)ethynyl)benzyl)-3-phenylurea;
2-(2-((1H-pyrrolo[2,3-b]pyridin-4-yl)ethynyl)phenyl)-N-(4-methoxybenzyl)acetamide;
2-(2-((1H-pyrrolo[2,3-b]pyridin-4-yl)ethynyl)phenyl)-N-tert-butylacetamide;
N-(2-((1H-pyrrolo[2,3-b]pyridin-4-yl)ethynyl)benzyl)-4-(dimethylamino)benzamide;
N-(2-((1H-pyrrolo[2,3-b]pyridin-4-yl)ethynyl)benzyl)-4-methoxybenzamide;
N-(2-((1H-pyrrolo[2,3-b]pyridin-4-yl)ethynyl)benzyl)-2,2,2-trifluoroacetamide;
2-(2-((1H-pyrrolo[2,3-b]pyridin-4-yl)ethynyl)phenyl)-N-(4-methoxyphenyl)acetamide;
2-(2-((1H-pyrrolo[2,3-b]pyridin-4-yl)ethynyl)phenyl)-1-(4,4-difluoropiperidin-1-yl)ethanone;
N-(2-((1H-pyrrolo[2,3-b]pyridin-4-yl)ethynyl)benzyl)-2,2,2-trifluoroacetamide;
1-(2-((1H-pyrrolo[2,3-b]pyridin-4-yl)ethynyl)benzyl)-3-tert-butylurea; and
3-((5,6,7,8-tetrahydro-1,8-naphthyridin-4-yl)ethynyl) aniline;
or a pharmaceutically acceptable salt thereof.

30-35. (canceled)

36. The compound of claim 1, 2, 3, 5, 25 or 29 for use as a medicament.

37. Use of the compound according to claim 1, 2, 3, 5, 25 or 29, for the preparation of a medicament for the treatment a subject in need of inhibition of a kinase protein.

38. The use according to claim 37 wherein the protein kinase is VEGFR3, VEGFR2, Flt-3, or PDK1.

39. The use according to claim 37 or 38, wherein the subject has a hyperproliferative disease or an inflammatory disease.

40. The use according to claim 39 wherein the hyperproliferative disease is selected from the group consisting of lymphoma, ovarian cancer, breast cancer, lung cancer, pancreatic cancer, prostate cancer, colon cancer and epidermoid cancer.

41. The use according to claim 39 wherein the subject has an inflammatory disease selected from the group consisting of multiple sclerosis, psoriasis, lung inflammation, systemic lupus erythermatosis, thrombosis, meningitis, encephalitis, rheumatoid arthritis, inflammatory bowel disease, and atherosclerosis.

42. Use of the compound according to claim 1, 2, 3, 5, 25 or 29, for the preparation of a medicament for the suppression of cancer metastasis in a subject in need thereof.

43-46. (canceled)
